(19) United States
(12) Patent Application Publication
(10) Pub. No.: US 2023/0357765 A1
(43) Pub. Date: Nov. 9, 2023

(54) USE OF LONG NON-CODING RNAS IN MEDULLOBLASTOMA

(71) Applicants: THE JOHNS HOPKINS UNIVERSITY, Baltimore, MD (US); UNIVERSITY OF CENTRAL FLORIDA RESEARCH FOUNDATION, INC., Orlando, FL (US)

(72) Inventors: Joseph Ranjan Perera, St. Petersburg, FL (US); Sudipta Seal, Oviedo, FL (US)

(21) Appl. No.: 18/026,222

(22) PCT Filed: Sep. 14, 2021
(86) PCT No.: PCT/US2021/050258

§ 371 Date: Mar. 14, 2023

Related U.S. Application Data
(60) Provisional application No. 63/077,967, filed on Sep. 14, 2020.

(51) Int. Cl. A61P 35/00; C12N 15/113

(52) U.S. Cl. CPC C12N 15/113; A61P 35/00; C12N 2310/113

(57) ABSTRACT

The present invention relates to the field of cancer. More specifically, the present invention provides compositions and methods useful for treating medulloblastoma. In one embodiment, a method for treating medulloblastoma in a patient comprises the step of administering a composition comprising an antisense oligonucleotides (ASO) targeting long non-coding ribonucleic acid HLX-2-7 (lnc-HLX-2-7). In particular embodiments, the medulloblastoma is group III medulloblastoma.

### CROSS-REFERENCE TO RELATED APPLICATIONS

This application claims the benefit of U.S. Provisional Application No. 63/077,967, filed Sep. 14, 2020, which is incorporated herein by reference in its entirety.

### FIELD OF THE INVENTION

The present invention relates to the field of cancer. More specifically, the present invention provides compositions and methods useful for treating medulloblastoma by targeting long non-coding RNAs (lncRNA).

### INCORPORATION-BY-REFERENCE OF MATERIAL SUBMITTED ELECTRONICALLY

This application contains a sequence listing. It has been submitted electronically via EFS-Web as an ASCII text file entitled “P16545-02_ST25.txt.” The sequence listing is 65,914 bytes in size, and was created on Sep. 14, 2021. It is hereby incorporated by reference in its entirety.

### BACKGROUND OF THE INVENTION

Medulloblastoma (MB), characterized as WHO group IV, represents the most common and highly malignant pediatric central nervous system tumor, representing 9.2% of all pediatric brain tumor cases and roughly 500 new cases of MB are annually diagnosed. MB are localized in the cerebellum, sharing signatures with embryonic cerebellar lineages, from where they commonly metastasize to other parts of the brain and spinal cord, and, rarely, to extraneural sites. Commonly used treatment strategies for MB, including maximal safe surgical resection, radiotherapy and chemotherapy, are aggressive for patients who are predominantly under 7 years of age. Appropriate treatment therapy selection depends upon clinical subgroup, stage, extent of resection and location, and patient’s ability to withstand the treatment. To aide treatment options a combinatorial genome wide sequencing, genetic alteration and DNA methylation approach has improved MB diagnosis into four clinically and molecularly distinct subgroup: wingless (WNT) sonic hedgehog (SHH), group 3 and group 4. Despite these significant advances in early diagnosis and effective treatment approaches, MB remains a deadly disease with around 30% fatality rate. Often eradication of tumor still results in deteriorated overall quality of life due to side effects including organ dysfunction, neurocognitive impairment, endocrine disabilities, and secondary tumors. In addition, even with advances in molecular classification, the defining molecular mechanism remains unknown in group 3 and group 4, making the proper diagnosis and treatment of the respective patient challenging. Hence, there is an urgent need to identify causative molecular mechanism to drive precision medicine based approaches that could improve the quality of life of patients and increase our understanding of MB in general.

### SUMMARY OF THE INVENTION

Medulloblastoma (MB) is an aggressive brain tumor that predominantly affects children. Recent high-throughput sequencing studies suggest that the noncoding RNA genome, in particular long noncoding RNAs (lncRNAs), contributes to MB subgrouping. Here we report the identification of a novel lncRNA, lnc-HLX-2-7, as a potential molecular marker and therapeutic target in Group 3 MBs.

Publicly available RNA sequencing (RNA-seq) data from 175 MB patients were interrogated to identify lncRNAs that differentiate between MB subgroups. After characterizing a subset of differentially expressed lncRNAs in vitro and in vivo, lnc-HLX-2-7 was deleted by CRISPR/Cas9 in the MB cell line. Intracranial injected tumors were further characterized by bulk and single-cell RNA-seq.

Lnc-HLX7 is highly upregulated in Group 3 MB cell lines, patient-derived xenografts, and primary MBs compared with other MB subgroups as assessed by quantitative real-time, RNA-seq, and RNA fluorescence in situ hybridization. Depletion of lnc-HLX-2-7 significantly reduced cell proliferation and 3D colony formation and induced apoptosis. Lnc-HLX-2-7-deleted cells injected into mouse cerebellums produced smaller tumors than those derived from parental cells. Pathway analysis revealed that lnc-HLX-2-7modulated oxidative phosphorylation, mitochondrial dysfunction, and sirtuin signaling pathways. The MYC oncogene regulated lnc-HLX-2-7, and the small-molecule bromodomain and extraterminal domain family-bromodomain 4 inhibitor Jun Qi 1 (JQ1) reduced lnc-HLX-2-7expression.

Lnc-HLX7 is oncogenic in MB and represents a promising novel molecular marker and a potential therapeutic target in Group 3 MBs.

Accordingly, in one aspect, the present invention provides compositions and methods for treating medulloblastoma. In one embodiment, a method for treating medulloblastoma in a patient comprises the step of administering a composition comprising an antisense oligonucleotide (ASO) targeting long non-coding ribonucleic acid HLX-2-7 (lnc-HLX-2-7). In particular embodiments, the medulloblastoma is group III medulloblastoma.

In certain embodiments, the ASO targets a 20-40 nucleotide sequence of lnc-HLX-2-7 (SEQ ID NO:200). In one embodiment, the ASO targets nucleotides 325-345 of SEQ ID NO:200. In a specific embodiment, the ASO comprises SEQ ID NO:242 or SEQ ID NO:290.

In another embodiment, the ASO targets nucleotides 335-361 of SEQ ID NO:200. In a specific embodiment, the ASO comprises SEQ ID NO:247 or SEQ ID NO:292. In an alternative embodiment, the ASO targets nucleotides 468-488 of SEQ ID NO:200. In a specific embodiment, the ASO comprises SEQ ID NO:240 or SEQ ID NO:289. In yet another embodiment, the ASO targets nucleotides 480-500 of SEQ ID NO:200. In a specific embodiment, the ASO comprises SEQ ID NO:244 or SEQ ID NO:291.

The present invention also provides a composition comprising an ASO that targets a 20-40 nucleotide sequence of lnc-HLX-2-7(SEQ ID NO:200). In particular embodiments, the 20-40 nucleotide sequence comprises nucleotides 110-132, nucleotides114-136, nucleotides 169-191, nucleotides 170-192, nucleotides174-196, nucleotides 176-198, nucleotides 183-205, nucleotides 211-233, nucleotides 220-242, nucleotides 222-244, nucleotides 275-297, nucleotides 276-298, nucleotides 321-343, nucleotides 323-345, nucleotides 335-345, nucleotides 331-353, nucleotides 333-355, nucleotides 335-361, nucleotides 350-372, nucleotides 352-374, nucleotides 466-488, nucleotides 468-488, nucleotides 480-500, or nucleotides 494-516.

In another embodiment, a method comprises the steps of (a) detecting overexpression of lnc-HLX-2-7in a sample obtained from a patient having medulloblastoma; and (b) treating the patient with a composition comprising a polymeric micelle and an ASO that targets lnc-HLX-2-7.

In yet another embodiment, the present invention provides a method comprising the step of administering a composition comprising a polymeric micelle and an ASO that targets lnc-HLX-2-7to a patient diagnosed with group III medulloblastoma. In certain embodiments, the method further comprises administering an additional therapeutic agent. In a specific embodiment, the therapeutic agent is cisplatin.

In another aspect, the present invention provides composition comprising antisense oligonucleotides (ASOs). In particular embodiments, a composition comprises an ASO that targets a 20-40 nucleotide sequence of lnc-HLX-2-7(SEQ ID NO:200). In more particular embodiments, the 20-40 nucleotide sequence comprises nucleotides 110-132, nucleotides114-136, nucleotides 169-191, nucleotides 170-192, nucleotides174-196, nucleotides 176-198, nucleotides 183-205, nucleotides 211-233, nucleotides 220-242, nucleotides 222-244, nucleotides 275-297, nucleotides 276-298, nucleotides 321-343, nucleotides 323-345, nucleotides 335-345, nucleotides 331-353, nucleotides 333-355, nucleotides 335-361, nucleotides 350-372, nucleotides 352-374, nucleotides 466-488, nucleotides 468-488, nucleotides 480-500, or nucleotides 494-516.

In more specific embodiments, the ASO targeting nucleotides 110-132 comprises SEQ ID NO:269, the ASO targeting nucleotides 114-136 comprises SEQ ID NO:270, wherein the ASO targeting nucleotides 169-191 comprises SEQ ID NO:271, wherein the ASO targeting nucleotides 170-192 comprises SEQ ID NO:272, wherein the ASO targeting nucleotides174-196 comprises SEQ ID NO:273, wherein the ASO targeting nucleotides 176-198 comprises SEQ ID NO:274, wherein the ASO targeting nucleotides 183-205 comprises SEQ ID NO:275, wherein the ASO targeting nucleotides 211-233 comprises SEQ ID NO:276, wherein the ASO targeting nucleotides 220-242 SEQ ID NO:277, wherein the ASO targeting nucleotides 222-244 comprises SEQ ID NO:278, wherein the ASO targeting nucleotides 275-297 comprises SEQ ID NO:279, wherein the ASO targeting nucleotides 276-298 comprises SEQ ID NO:280, wherein the ASO targeting nucleotides 321-343 comprises SEQ ID NO:281, wherein the ASO targeting nucleotides 323-345 comprises SEQ ID NO:282, wherein the ASO targeting nucleotides 331-353 comprises SEQ ID NO:283, wherein the ASO targeting nucleotides 333-355 comprises SEQ ID NO:284, wherein the ASO targeting nucleotides 350-372 comprises SEQ ID NO:285, wherein the ASO targeting nucleotides 352-374 comprises SEQ ID NO:286, wherein the ASO targeting nucleotides 466-488 comprises SEQ ID NO:287, or wherein the ASO targeting nucleotides comprises SEQ ID NO:288.

In other embodiments, the 20-40 nucleotide sequence comprises nucleotides 325-345, nucleotides 335-361, nucleotides 468-488 or nucleotides 480-500. In specific embodiments, the ASO targeting nucleotides 325-345 comprises SEQ ID NO:242, wherein the ASO targeting nucleotides 335-361 comprises SEQ ID NO:247, wherein the ASO targeting nucleotides 468-488 comprises SEQ ID NO:240 or wherein the ASO targeting nucleotides 480-500 comprises SEQ ID NO:244.

It is understood that the ASO compositions described herein include not only the sequence listed herein and the sequence, but also can include phosphorothioate (PS) linkages and/or locked nucleic acids (LNAs). Examples of such ASOs are described herein.

The ASOs described in SEQ ID NOS:269-288 can include, for example, PN linkages at amino acid positions 1-22, 1-23, 2-22, 2-23, and, as well as, aa 1-18, 1-19, 1-20, 1-21, 1-22, 1-23, 2-18, 2-19, 2-20, 2-21, 2-23. The ASOs described in SEQ ID NOS:269-288 can also include, for example, LNA at amino acid positions 1-2, 1-3, 1-4, 1-5, 2-3, 2-4, 2-5, 3-4, 3-5, as well as 20-23, 21-23, 22-23, 19-23, 19-22, 19-21, 19-20, 20-22, 18-23, 18-22, and 18-21.

The compositions of the present invention can further comprise a polymeric micelle. In more specific embodiments, the polymeric micelle comprises a cerium oxide nanoparticle. In particular embodiments, the present invention provides methods comprising creating mixed valence state of cerium oxide nanoparticle for ASO conjugation. Such methods include, for example, controlling +3/+4 ratio for ASO- and related conjugation. In particular embodiments, the surface charge of the cerium nanoparticles are modified to encapsulate the polymeric micelle. In other embodiments, the surface charge of ASO-conjugated cerium oxide nanoparticles are modified to encapsulate the polymeric micelle. In certain embodiments, it is understood that as the nucleotide sequence of the ASO changes, then the cerium oxide nanoparticle surface is also modified.

### BRIEF DESCRIPTION OF THE FIGURES

FIGS. 1A-1F. Identification and validation of the Group 3-specific lncRNA, lnc-HLX-2-7. FIG. 1(A) Schematic of the identification of Group 3-specific lncRNAs in the 4 MB subgroups (WNT, SHH, Group 3 and Group 4). (FIG. 1B) Top 50 lncRNAs with the highest expression in Group 3 MBs compared with other MB subgroups are shown. x-axis indicates P value (-log10) of each lncRNA and y-axis indicates fold change value (log2) of each lncRNA. (FIG. 1C) The heat map represents the similarity of expression within Group 3 MBs of each lncRNA shown in (FIG. 1B). (FIG. 1D) Boxplot showing distribution of normalized expression values of lnc-HLX-1, lnc-HLX-2, Inc-HLX-5, and lnc-HLX-6 in WNT, SHH, Group 3 and Group 4 MBs. Dots represent the expression value for each MB patient. *P < 0.01, Kruskal-Wallis analysis. (FIG. 1E, FIG. 1F) qRT-PCR analysis showing the distribution of normalized expression values of lnc-HLX-2-7in MB cell lines (FIG. 1E) and PDX samples (FIG. 1F) of Group 3, Group 4, and SSH MBs. Values indicate fold change relative to cerebellum.

FIGS. 2A-2F: Effects of lnc-HLX-2-7expression on the proliferation and apoptosis of Group 3 MB cells. (FIG. 2A) Expression level of lnc-HLX-2-7 in D425 Med and MED211 cells treated with ASO against the genes indicated on the x-axis. Relative expression level to mock (non-transfected) is indicated on the y-axis. *P < 0.01, Kruskal-Wallis analysis. Viable cell numbers (FIG. 2B) and apoptotic cell numbers (FIG. 2C) in D425 Med and MED211 cells treated with either ASO-luc or ASO- lnc-HLX-2-7. Relative value to mock is indicated on the y-axis. *P < 0.01, Kruskal-Wallis analysis. (FIG. 2D) Expression level of lnc-HLX-2-7 in D425 Med and MED211 control (CTRL) and D425 Med and MED211-lnc-HLX-2-7-sgRNA (lnc-HLX-2-7) cells. Relative expression level to CTRL is indicated on the y-axis. *P < 0.01, Student’s t-test. (FIG. 2E) Cell viability assays performed with D425 Med and MED211 control (CTRL) and D425 Med and MED211-lnc-HLX-2-7-sgRNA (lnc-HLX-2-7) cells. Points represent the mean and standard deviation of 3 biological replicates. *P < 0.01, Student’s t-test. (FIG. 2F) Colony formation assays performed with D425 Med and MED211 control (CTRL) and D425 Med and MED211-lnc-HLX-2-7-sgRNA (lnc-HLX-2-7) cells. 3 independent experiments were performed, and data are presented as mean ± SD. *P < 0.01, Student’s t-test.

FIGS. 3A-3E. Lnc-HLX-2-7 promotes the tumorigenicity of Group 3 MB cells in vivo. (FIG. 3A) D425 Med and MED211 control (CTRL) and D425 Med- and MED211-lnc-HLX-2-7-sgRNA (lnc-HLX-2-7) cells expressing luciferase were implanted into the right forebrains of NOD-SCID mice, and tumor formation was assessed by bioluminescence imaging. Changes in bioluminescent signal were examined weekly after tumor implantation. (FIG. 3B) Quantification of total photon counts from mice implanted with D425 Med and MED211 control (CTRL) and D425 Med- and MED211-lnc-HLX-2-7- sgRNA (lnc-HLX-2-7) cells. n = 9, *P < 0.05, Student’s t-test. (FIG. 3C) Ki67 and (FIG. 3D) TUNEL staining of xenograft tumors. Nuclei are stained with DAPI. Scale bars, 50 µm. Quantification of Ki67 and TUNEL-positive cells were shown. *P < 0.05, Student’s t-test. (FIG. 3E) Overall survival was determined by Kaplan-Meier analysis, and the log-rank test was applied to assess the differences between groups. *P < 0.05, Mantel-Cox log-rank test.

FIGS. 4A-4D. MYC regulates the expression of lnc-HLX-2-7 in Group 3 MB. (FIG. 4A) Expression levels of MYC and lnc-HLX-2-7 in D425 Med and MED211 cells treated with siRNA against the indicated genes on the x-axis. Relative expression level to mock (non-transfected) is indicated on the y-axis. *P < 0.01, Kruskal-Wallis analysis. (FIG. 4B) Schematic diagram showing E-box motifs around the TSS of lnc-HLX-2-7. Open circles indicate E-box motifs. Arrows show the primer location of ChIP-qPCR. (FIG. 4C) Enrichment of MYC in the lnc-HLX-2-7 promoter regions in DAOY, D425 Med, and MED211 cells. Enrichment is expressed as a percentage of input DNA. *P < 0.01, Student’s t-test. (FIG. 4D) Expression level of MYC and lnc-HLX-2-7 in D425 Med, and MED211 cells treated with JQ1. Values are indicated relative to abundance in DMSO-treated cells. *P < 0.01, Kruskal-Wallis analysis.

FIGS. 5A-5G. RNA sequencing detects lnc-HLX-2-7 interacting genes and pathways. (FIG. 5A) Heatmap representation of genes up and downregulated after lnc-HLX-2-7 depletion in D425 xenografts. (FIG. 5B) Molecular and cellular functions and diseases associated with these genes. (FIG. 5C) IPA Canonical Pathway analysis was performed to predict signaling pathway activity. The 10 most significant pathways with lowest P values are presented. (FIG. 5D) Uniform Manifold Approximation and Projection (UMAP) plot of transcriptionally distinct cell populations from aggregate CTRL and lnc-HLX-2-7-deleted xenograft scRNA-seq samples. Five distinct clusters (1-5) were identified. Marker genes associated with each cluster are listed in Supplementary Table 5 (available online). (FIG. 5E) UMAP plot with CTRL and lnc-HLX-2-7-deleted xenograft samples highlighted. Bar chart indicates the percentage of cells from each xenograft sample for the clusters corresponding to (FIG. 5D). (FIG. 5F) IPA Canonical Pathway analysis to predict signaling pathway activity in clusters 1, 2, 3, 4, and 5. The top canonical pathways with lowest adjusted P values are shown. (FIG. 5G) Pseudotemporal trajectory of cells from CTRL to lnc-HLX-2-7-deleted cells. Numbered circle with white background denotes the root node selected for pseudotemporal ordering, black circles represent branch nodes (where cells can proceed to different outcomes), and gray circles indicate different outcomes. The red trajectory denotes the structure of pseudotime graph. Cell colors denote the progression of cells along pseudotime.

FIGS. 6A-6E. RNA-FISH confirms that lnc-HLX-2-7 expression is specific to Group 3 MB patients. (FIG. 6A) Representative RNA-FISH analysis of lnc-HLX-2-7 and MYC in MB tissues. RNA-FISH analysis of lnc-HLX-2-7and MYC in Group 3 MB patients (upper panels) and Group 4 MB patients (lower panels). (FIG. 6B) Representative RNA-FISH analysis of lnc-HLX-2-7 and MYCN in MB tissues. RNA-FISH analysis of lnc-HLX-2-7and MYCN in Group 3 MB patients (upper panels) and Group 4 MB patients (lower panels). Nuclei were stained with DAPI. Scale bars, 10 µm. (FIG. 6C) The spot numbers relating to lnc-HLX-2-7, MYC, and MYCN were quantified per cell in Group 3 and Group 4 MB patients. n = 20, *P < 0.01, Student’s t-test. (FIG. 6D) Correlation between lnc-HLX-2-7and MYC expression in Group 3 MB patients. n = 20, *P < 0.01, Pearson correlation coefficient. (FIG. 6E) Kaplan-Meier survival curves of Group 3 MB patients according to lnc-HLX-2-7and MYC expression. n = 10, *P < 0.01, log-rank test.

FIGS. 7A-7D. Location of HLX and lnc-HLX-2-7and expression levels of lnc-HLX-2-7 variants. (FIG. 7A) lnc-HLX-2-7is a 517 bp intronic lncRNA encoded within the HLX gene located 2300 bp downstream of the HLX gene. The fourth and the fifth exons of the lnc-HLX-2-7 are repeated elements. The first exon has a 32 bp repeat at its end, while the second and third exons are non-repeated. (FIG. 7B) lnc-HLX-2 contains 11 transcripts (lnc-HLX-2-1 to Inc-HLX-2-11). (FIG. 7C) Boxplot showing distribution of normalized expression values of 11 transcripts (lnc-HLX-2-1 to Inc-HLX-2-11) of lnc-HLX-2 in group 3 MBs. *p<0.01, Kruskal-Wallis analysis. (FIG. 7D) Boxplot showing distribution of normalized expression values of lnc-HLX-2-7 in the eight molecular subtypes of group 3 and group 4 MB. Dots represent the expression value for each MB patient. *p<0.01, Kruskal-Wallis analysis.

FIG. 8. lnc-HLX-2-7regulates the expression of HLX coding gene. Expression levels of HLX in D425 Med and MED211 cells treated with ASO against the indicated genes in the x-axis. Relative expression level to mock is indicated in the y-axis. *p<0.01, Kruskal-Wallis analysis.

FIGS. 9A-9B. Effects of HLX expression on the proliferation of D425 Med and MED211. (FIG. 9A) Expression levels of HLX in D425 Med and MED211 cells treated with siRNA against the indicated genes in the x-axis. (FIG. 9B) Viable cell numbers in D425 Med and MED211 cells treated with either si-NC or si-HLX. Relative value to mock is indicated in the y-axis. *p<0.01, Kruskal-Wallis analysis.

FIGS. 10A-10C. JQ1 regulates lnc-HLX-2-7 via MYC in vivo. (FIG. 10A) D425 Med and MED211 cells expressing luciferase were implanted into the right forebrains of NOD-SCID mice. Seven days after injection, mice were administered DMSO or JQ1. Tumor formation was assessed by bioluminescence imaging. Changes in bioluminescent signal were examined weekly after tumor implantation. (FIG. 10B) Quantification of total photon counts from mice treated with JQ1 or DMSO. n=4, *p<0.05, Student’s t-test. (FIG. 10C) Expression levels of MYC and lnc-HLX-2-7 were examined by qPCR in DMSO or JQ1-treated mouse xenografts. Relative expression levels compared to those in the DMSO-treated tumor are indicated on the y-axis (n=4). Error bars indicate s.e.m. n=4, *p<0.01, Student’s t-test.

FIGS. 11A-11C. Overexpression of lnc-HLX-2-7rescued cell growth inhibition and downregulation of MYC by JQ1. (FIG. 11A) Expression levels of lnc-HLX-2-7in pcDNA4 or pcDNA4-lnc-HLX-2-7-expressing D425 Med and MED211 cells treated with JQ1. Relative value to pcDNA4 is indicated in the y-axis. *p<0.01, Student’s t-test. (FIGS. 11B, 11C) Viable cell numbers (FIG. 11B) and expression level of MYC (FIG. 11C) in pcDNA4 or pcDNA4-lnc-HLX-2-7-expressing D425 Med and MED211 cells treated with JQ1. Relative value to DMSO is indicated in the y-axis. *p<0.01, Kruskal-Wallis analysis.

FIGS. 12A-12C. lnc-HLX-2-7interacting pathway genes in D425 Med cells. (FIG. 12A) Heatmap representation of genes up- and downregulated after lnc-HLX-2-7 depletion in D425 Med cells (p<0.05). (FIG. 12B) Molecular and cellular functions and diseases associated with these genes. (FIG. 12C) The most significant upstream regulators inhibited by depletion of lnc-HLX-2-7.

FIG. 13. qPCR validation of D425 Med xenograft RNA-sequencing data. Expression levels of lnc-HLX-2-7, PTGR1, FDZ6, TRPM, NAMPT, NRBP2, NBAT1, CCNG2, ELK4, CDKN2C, CDK6, SOX4, CHD7, MYC, ETC2, NME7, GRM5-AS1, MYBPH, GPR158-AS1, NCAM1-AS1, KANTR, POTEI, ZEB2-AS1, and NR1D1 were examined by qPCR in D425 Med xenografts. Relative expression levels compared with those in the CTRL tumors are indicated on the y-axis (n=3). Error bars indicate s.e.m. *p<0.01, Student’s t-test.

FIG. 14. Boxplots showing the distribution of percentage of reads emanating from mitochondrial genes before and after filtering cells based on mitochondrial content. Cells were filtered for <10% mitochondrial percentage prior to analysis using Seurat and Monocle3.

FIG. 15. Graph path corresponding to transition of cells from cluster 1 through 5. Selected cells (in purple) along a selected trajectory for pseudotemporal graph test to determine significant genes that vary along the chosen path. The UMAP space corresponds to FIG. 5D.

FIGS. 16A-16B. Confirmation of the specificity of the lnc-HLX-2-7probe. (FIG. 16A) Representation of RNA-FISH analysis of lnc-HLX-2-7 and MYC in MB tissues. RNA-FISH analysis of lnc-HLX-2-7 and MYC in normal mouse brains (upper panels) and D425 Med xenografts (lower panels). Nuclei are stained with DAPI. Scale bars, 10 µm. The spot numbers relating to lnc-HLX-2-7 and MYC were quantified per cell in normal mouse brain and D425 Med xenograft. *p<0.01.

FIGS. 17A-17C. RNA-FISH confirms that lnc-HLX-2-7 is not expressed in SHH MB patients. RNA-FISH analysis of lnc-HLX-2-7and MYC (FIG. 17A) or MYCN (FIG. 17B) in SHH MB tissues. Nuclei are stained with DAPI. Scale bars, 10 µm. (FIG. 17C) The spot numbers relating to lnc-HLX-2-7, MYC, and MYCN were quantified per cell in Group 3, Group 4, and SHH MB patients. n=20, *p<0.01.

FIG. 18. Expression analysis of lnc-HLX-2-7and MYC in clinical MB samples. Correlation between lnc-HLX-2-7and MYC expression in clinical MB samples. Data were obtained from RNA sequencing data from 175 MB patients (ICGC). Each comparison is performed between the genes indicated on the x- and y-axes, respectively.

FIG. 19. Spry4-IT1 (“SPRIGHTLY”). RNA is expressed in medulloblastoma group 4 patient samples, but not in group 3. Sprightly (red), MYCN (green) are visualized in FFPE samples in group 4, but not in group 3. DAPI (blue) is stained to depict the nuclei. The control does not show the expression of either Sprightly or MYCN.

FIG. 20. Overview of polymeric micelle containing ASO-HLX-2-7.

FIG. 21. Analysis of cerium oxide nanoparticle (CNP)-HLX-2-7 accumulation in mouse brain tumor. Confirmation of tumor formation (LUC activity) by IVIS.

FIG. 22. Analysis of CNP-HLX-2-7 accumulation in mouse brain tumor. Intravenous administration to mice and detection of signal (Alexa647) by IVIS.

FIG. 23. Analysis of CNP-HLX-2-7 accumulation in mouse brain tumor. 6h, 9h, 12h, 24h, and 48h after administration.

FIG. 24. Analysis of anti-tumor effect of HLX-2-7. Day 0 and 1^{st} injection.

FIG. 25. Analysis of anti-tumor effect of HLX-2-7. Day 3 and 2^{nd} injection.

FIG. 26. Analysis of anti-tumor effect of HLX-2-7. Day 6 and 3^{rd} injection.

FIG. 27. Analysis of anti-tumor effect of HLX-2-7. Day 9 and 4^{th} injection.

FIG. 28. Analysis of anti-tumor effect of HLX-2-7. Day 12 and 5^{th} injection.

FIG. 29. Analysis of anti-tumor effect of HLX-2-7. Day 15 and 6^{th} injection.

FIG. 30. Analysis of anti-tumor effect of HLX-2-7. Day 18 and 7^{th} injection.

FIG. 31. Analysis of anti-tumor effect of HLX-2-7. Day 21.

FIG. 32. Analysis of anti-tumor effect of HLX-2-7. Day 24, Day 27 and Day 30.

FIG. 33. Analysis of anti-tumor effect of HLX-2-7. Day 33, Day 36 and Day 39.

FIG. 34. Expression analysis of HLX-2-7 in ASO-treated mice. lncHLX-2-7/ACTB.

FIG. 35. Expression analysis of HLX-2-7 in ASO-treated mice. HLX/ACTB.

FIG. 36. Expression analysis of HLX-2-7 in ASO-treated mice. MYC/ACTB.

FIG. 37. Analysis of lnc-HLX-2-7inhibition. Expression of analysis of lnc-HLX-2-7.

FIG. 38. CNP conjugated ASO. Analysis of incorporation of ASO into cells.

FIG. 39. CNP conjugated ASO3. Expression analysis of lnc-HLX-2-7.

FIG. 40. Drug delivery system (DDS) using polymeric micelle.

FIG. 41. Overview of polymeric micelle containing ASO-HLX-2-7.

FIG. 42. Analysis of anti-tumor effect of HLX-2-7.

FIG. 43. Expression analysis of HLX-2-7 in ASO-treated mice.

FIG. 44. Expression analysis of HLX-2-7 in ASO-treated mice.

FIG. 45. Expression analysis of HLX-2-7 in ASO-treated mice.

FIG. 46. Analysis of cell growth inhibitory effect by Cisplatin.

FIGS. 47A-47D. Analysis of anti-tumor effect of ASO-lnc-HLX-2-7. (FIG. 47A) D425 Med cells, expressing luciferase were implanted into the cerebellum of NOD-SCID mice. After 14 days of transplantation, CNP-CTRL or CNP-lnc-HLX-2-7 were intravenous injected every three days for 3 weeks (total 8 injections for 21 days). (FIG. 47B) Tumor formation was assessed by bioluminescence imaging. Changes in bioluminescent signal were examined every three days after 1st treatment. (FIG. 47C) Quantification of total photon counts from mice during the treatment. n=10, *p<0.05, Student’s t-test. (FIG. 47D) Overall survival was determined by Kaplan-Meier analysis, and the log-rank test was applied to assess the differences between groups. n=10, *p<0.05, Mantel-Cox log-rank test.

FIGS. 48A-48C. Confirmation of ASO design and knockdown effect of lnc-HLX-2-7. (FIG. 48A) Diagram showing the sites targeted by each ASO. (FIGS. 48B-48C) Expression level of lnc-HLX-2-7in D425 Med (FIG. 48B) and MED211 (FIG. 48C) cells treated with ASO against the indicated genes on the x-axis. ASOs were transfected at 50 nM for 72 h using Lipofectamine 3000 (Thermo Fisher Scientific, Waltham MA). The efficiency was determined by qRT-PCR. Relative expression level to luciferase (Luc) gene is indicated on the y-axis. *p<0.01, Kruskal-Wallis analysis.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Accordingly, in one aspect, the present invention provides compositions and methods for treating medulloblastoma. In specific embodiments, the present invention provides compositions and methods for treating Group III medulloblastoma. In more specific embodiments, the compositions and methods of the present invention comprise antisense oligonucleotides (ASO) directed at lnc-HLX-2-7. The treatment methods of the present invention can further comprise a detection step, as described herein.

More specifically, ASO can be designed to knock-out expression of lnc-HLX-2-7. In other embodiments, ASO can be designed to reduce expression lnc-HLX-2-7to treat Group III MB. lnc-HLX-2-7is known in the art:
LNCipedia transcript ID: lnc-HLX-2:7
LNCipedia gene ID: lnc-HLX-2
Location (hg38): chr1:220881701-220904006
Strand: +
Class: sense-overlapping
Sequence Ontology term: sense overlap ncRNA
Transcript size: 517 bp
Exons: 5
Sources: NONCODE v4
Alternative transcript names: NONHSAT009630

The RNA sequence of lnc-HLX-2-7is as follows:

atgacctcaaacacttgtgcttggcgagtttatgtctgggtgcctggaca
catgcgggaataaacacacacacacacacacacacacacacacattcgat
gttactgcattccttcatccattcatttttttcattgcagtatttatggg
gatccttgtgagtgtttgcaccatagagaaaaaagtatttgactaagcat
taaccattccagctaagagatcagtgtttgtcattcaaaatagctgaggt
ggttgggagaggacagccagtattccccaaaagaggtttattagtttcct
aggctgctgtcacaaattgacacagacttagtgacttaaaacaacagaaa
tgtgtttgtgtcactacattctctgcttctctggcacatggactcttctt
ctgcatccagttttcctctgtcttcctctcataagcatgcgtgtggtagc
atttagcgtccacccaggcaatctagattaatctctcacctcaagctcct taactgaatcacatctg (SEQ ID NO:200) (double underlin
eand italics, see below)

Antisense oligonucleotides useful in the present invention include the following:
(ASO1) lncHLX-2-7-1; +G^{∗}+T^{∗}+T^{∗}G^{∗}T^{∗}T^{∗}T^{∗}T^{∗}A^{∗}A^{∗}G^{∗}T^{∗}C^{∗}A^{∗}C^{∗}T^{∗}A^{∗}A^{∗}+G^{∗}+T^{∗}+C (SEQ ID NO:242);
Target site ; GACTTAGTGACTTAAAACAAC (SEQ ID NO:243) (nucleotides 325-345 of SEQ ID NO:200)
(ASO2) ASO-lncHLX-2-7-2; +A^{∗}+G^{∗}+G^{∗}A^{∗}G^{∗}C^{∗}T^{∗}T^{∗}G^{∗}A^{∗}G^{∗}G^{∗}T^{∗}G^{∗}A^{∗}G^{∗}A^{∗}G^{∗}+A^{∗}+T^{∗}+T (SEQ ID NO:244)
Target site ; AATCTCTCACCTCAAGCTCCT (SEQ ID NO:245) (nucleotides 480-500 of SEQ ID NO:200)
(ASO3) ASO-lncHLX-2-7-3; +T^{∗}+G^{∗}+A^{∗}G^{∗}A^{∗}G^{∗}A^{∗}T^{∗}T^{∗}A^{∗}A^{∗}T^{∗}C^{∗}T^{∗}A^{∗}G^{∗}A^{∗}T^{∗}+T^{∗}+G^{∗}+C (SEQ ID NO:240)
Target site ; GCAATCTAGATTAATCTCTCA (SEQ ID NO:246) (nucleotides 468-488 of SEQ ID NO:200)
(ASO4) ASO-lncHLX-2-7-4; +A*+C*+A*C*A*T*T*T*C*T*G*T*T*G*T*T*T*T*+A*+A*+G (SEQ ID NO:247)
Target site ; CTTAAAACAACAGAAATGTGT (SEQ ID NO:248) (nucleotides 335-361 of SEQ ID NO:200)
(+N=LNA, *=Phosphorothioated). It is understood that the ASO compositions described herein include not only the sequence listed herein and in the sequence listing, but also can include phosphorothioate (PS) linkages and/or locked nucleic acids (LNAs). Examples of such ASOs are described above. Thus, in particular embodiments, the ASOs in SEQ ID NOS:240, 242, 244 and 247 can include PN linkages at amino acid positions 1-21, 1-20, 2-21, 2-20, and, as well as, aa 1-18, 1-19, 1-20, 1-21, 2-18, 2-19, 2-20, 2-21, 3-18, 3-19, 3-20, 3-21, and so forth. The ASOs in SEQ ID NOS:240, 242, 244 and 247 can include LNAs at amino acid positions 1-2, 1-3, 1-4, 1-5, 2-3, 2-4, 2-5, 3-4, 3-5, as well as 18-21, 18-20, 19-20, 17-21, 17-20, 17-19, 16-21, 16-20, 16-19, 16-18, 16-17, and so forth.

Thus, in particular embodiments a composition of the present invention comprises ASO1 (SEQ ID NO:242) or SEQ ID NO :290. These sequences are identical ; however, SEQ ID NO :242 describes a particular embodiment of the ASO with PN linkages and LNA. In other embodiments, a composition of the present invention comprises ASO2 (SEQ ID NO:244) or SEQ ID NO :291. These sequences are identical ; however, SEQ ID NO :244 describes a particular embodiment of the ASO with PN linkages and LNA. In alternative embodiments, a compositions of the present invention comprises ASO3 (SEQ ID NO:240) or SEQ ID NO :289. These sequences are identical ; however, SEQ ID NO :240 describes a particular embodiment of the ASO with PN linkages and LNA. In further embodiments, a composition of the present invention comprises ASO4 (SEQ ID NO:247) or SEQ ID NO :292. These sequences are identical ; however, SEQ ID NO :247 describes a particular embodiment of the ASO with PN linkages and LNA.

In certain embodiments, a composition of the present invention comprises an ASO that targets SEQ ID NO:243 (nucleotides 325-345 of SEQ ID NO:200). In other embodiments, a composition comprises an ASO that targets SEQ ID NO:245 (nucleotides 480-500 of SEQ ID NO:200). In further embodiments, a composition comprises an ASO that targets SEQ ID NO:246 (nucleotides 468-488 of SEQ ID NO:200). In certain embodiments, a composition comprises an ASO that targets SEQ ID NO:248 (nucleotides 335-361 of SEQ ID NO:200).

A composition of the present invention can comprise at least one ASO directed at lnc-HLX-2-7, including, but not limited to, ASO1, ASO2, ASO3 and AS04.

In other embodiments, the present invention provides compositions and methods directed to ASOs that target other regions of lnc-HLX-2-7RNA (SEQ ID NO:200) including, but not limited to target positions: 110-132 (TTCCTTCATCCATTCATTTTTTT) (SEQ ID NO:249); 114-136 (TTCATCCATTCATTTTTTTCATT) (SEQ ID NO:250); 169-191

(CACCATAGAGAAAAAAGTATTTG) (SEQ ID NO:251);170-192

(ACCATAGAGAAAAAAGTATTTGA) (SEQ ID NO:252); 174-196

(TAGAGAAAAAAGTATTTGACTAA) (SEQ ID NO:253); 176-198

(GAGAAAAAAGTATTTGACTAAGC) (SEQ ID NO:254); 183-205

(AAGTATTTGACTAAGCATTAACC) (SEQ ID NO:255); 211-233

(AGCTAAGAGATCAGTGTTTGTCA) (SEQ ID NO:256); 220-242

(ATCAGTGTTTGTCATTCAAAATA) (SEQ ID NO:257); 222-244

(CAGTGTTTGTCATTCAAAATAGC) (SEQ ID NO:258); 275-297

(CCCCAAAAGAGGTTTATTAGTTT) (SEQ ID NO:259); 276-298

(CCCAAAAGAGGTTTATTAGTTTC) (SEQ ID NO:260); 321-343

(CACAGACTTAGTGACTTAAAACA) (SEQ ID NO:261); 323-345

(CAGACTTAGTGACTTAAAACAAC) (SEQ ID NO:262); 331-353

(GTGACTTAAAACAACAGAAATGT) (SEQ ID NO:263); 333-355

(GACTTAAAACAACAGAAATGTGT) (SEQ ID NO:264); 350-372

(ATGTGTTTGTGTCACTACATTCT) (SEQ ID NO:265); 352-374

(GTGTTTGTGTCACTACATTCTCT) (SEQ ID NO:266); 466-488

(AGGCAATCTAGATTAATCTCTCA) (SEQ ID NO:267); and 494
-516

(AGCTCCTTAACTGAATCACATCT) (SEQ ID NO:268).

In one embodiment, an ASO that targets SEQ ID NO:249 comprises AAAAAAATGAATGGATGAAGGAA (SEQ ID NO:269). In another embodiment, an ASO that targets SEQ ID NO:250 comprises AATGAAAAAAATGAATGGATGAA (SEQ ID NO:270). In further embodiments, an ASO that targets SEQ ID NO:251 comprises CAAATACTTTTTTCTCTATGGTG (SEQ ID NO:271). An ASO that targets SEQ ID NO:252 comprises TCAAATACTTTTTTCTCTATGGT (SEQ ID NO:272). An ASO that targets SEQ ID NO:253 comprises TTAGTCAAATACTTTTTTCTCTA (SEQ ID NO:273). In another embodiment, an ASO that targets SEQ ID NO:254 comprises GCTTAGTCAAATACTTTTTTCTC (SEQ ID NO:274). An ASO that targets SEQ ID NO:255 comprises GGTTAATGCTTAGTCAAATACTT (SEQ ID NO:275).

In one embodiment, an ASO that targets SEQ ID NO:256 comprises TGACAAACACTGATCTCTTAGCT (SEQ ID NO:276). In another embodiment, an ASO that targets SEQ ID NO:257 comprises TATTTTGAATGACAAACACTGAT (SEQ ID NO:277. In further embodiments, an ASO that targets SEQ ID NO:258 comprises GCTATTTTGAATGACAAACACTG (SEQ ID NO:278). An ASO that targets SEQ ID NO:259 comprises, for example, AAACTAATAAACCTCTTTTGGGG (SEQ ID NO:279). In yet another embodiment, an ASO that targets SEQ ID NO:260 comprises GAAACTAATAAACCTCTTTTGGG (SEQ ID NO:280). An ASO that targets SEQ ID NO:261 comprises, for example, TGTTTTAAGTCACTAAGTCTGTG (SEQ ID NO:281). In an alternative embodiment, an ASO that targets SEQ ID NO:262 comprises GTTGTTTTAAGTCACTAAGTCTG (SEQ ID NO:282).

In one embodiment, an ASO that targets SEQ ID NO:263 comprises ACATTTCTGTTGTTTTAAGTCAC (SEQ ID NO:283). In another embodiment, an ASO that targets SEQ ID NO:264 comprises ACACATTTCTGTTGTTTTAAGTC (SEQ ID NO:284. In further embodiments, an ASO that targets SEQ ID NO:265 comprises AGAATGTAGTGACACAAACACAT (SEQ ID NO:285). An ASO that targets SEQ ID NO:266 comprises, for example, AGAGAATGTAGTGACACAAACAC (SEQ ID NO:286). In yet another embodiment, an ASO that targets SEQ ID NO:267 comprises TGAGAGATTAATCTAGATTGCCT (SEQ ID NO:287). An ASO that targets SEQ ID NO:268 comprises, for example, AGATGTGATTCAGTTAAGGAGCT (SEQ ID NO:288).

The ASOs described in SEQ ID NOS:269-288 can include, for example, PN linkages at amino acid positions 1-22, 1-23, 2-22, 2-23, and, as well as, aa 1-18, 1-19, 1-20, 1-21, 1-22, 1-23, 2-18, 2-19, 2-20, 2-21, 2-23. The ASOs described in SEQ ID NOS:269-288 can also include, for example, LNA at amino acid positions 1-2, 1-3, 1-4, 1-5, 2-3, 2-4, 2-5, 3-4, 3-5, as well as 20-23, 21-23, 22-23, 19-23, 19-22, 19-21, 19-20, 20-22, 18-23, 18-22, and 18-21.

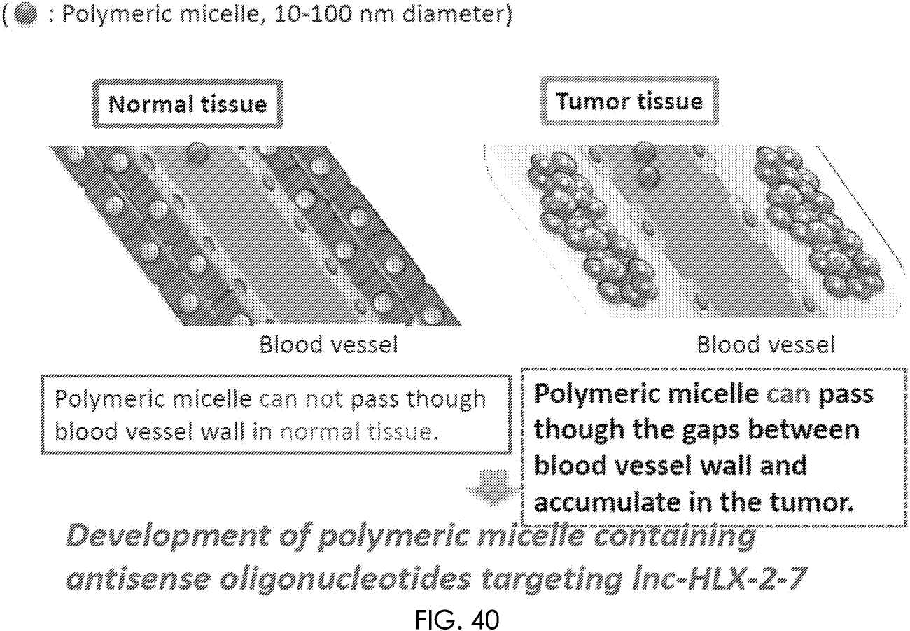

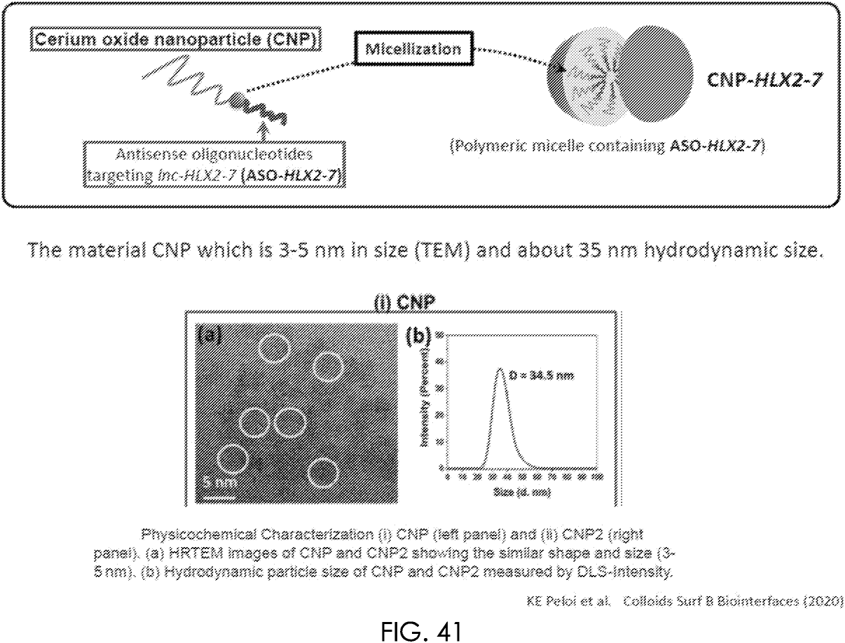

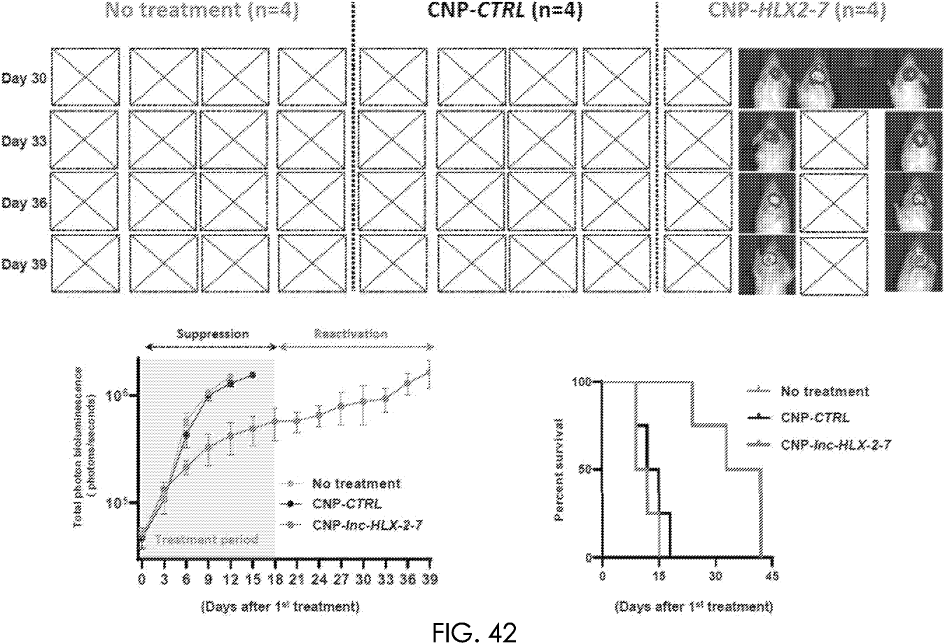

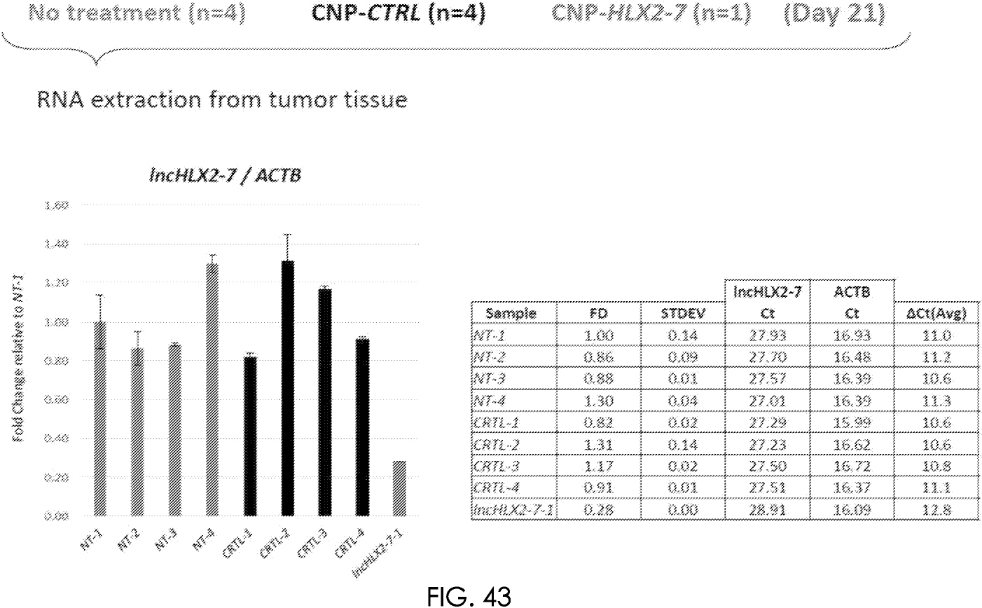

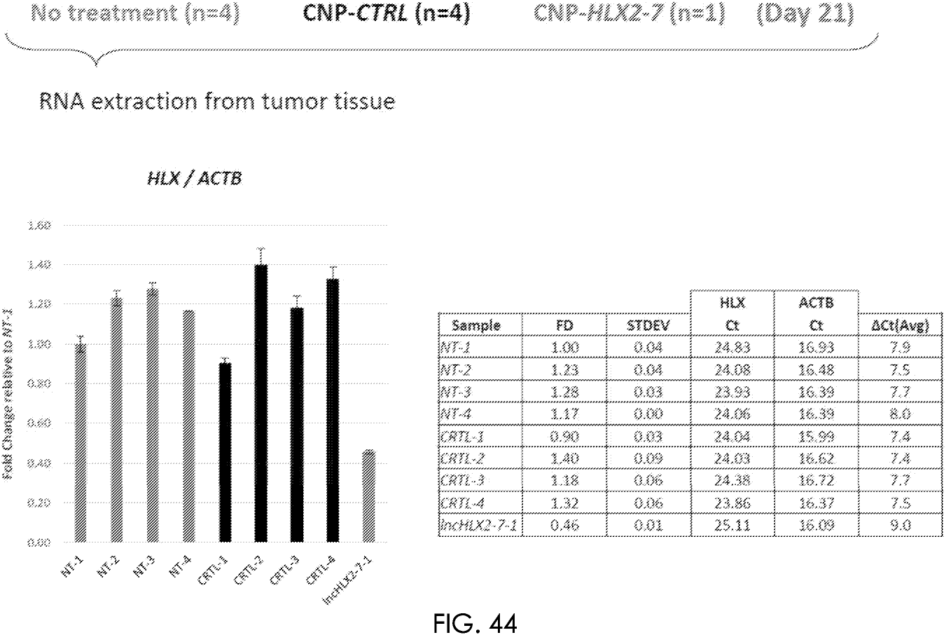

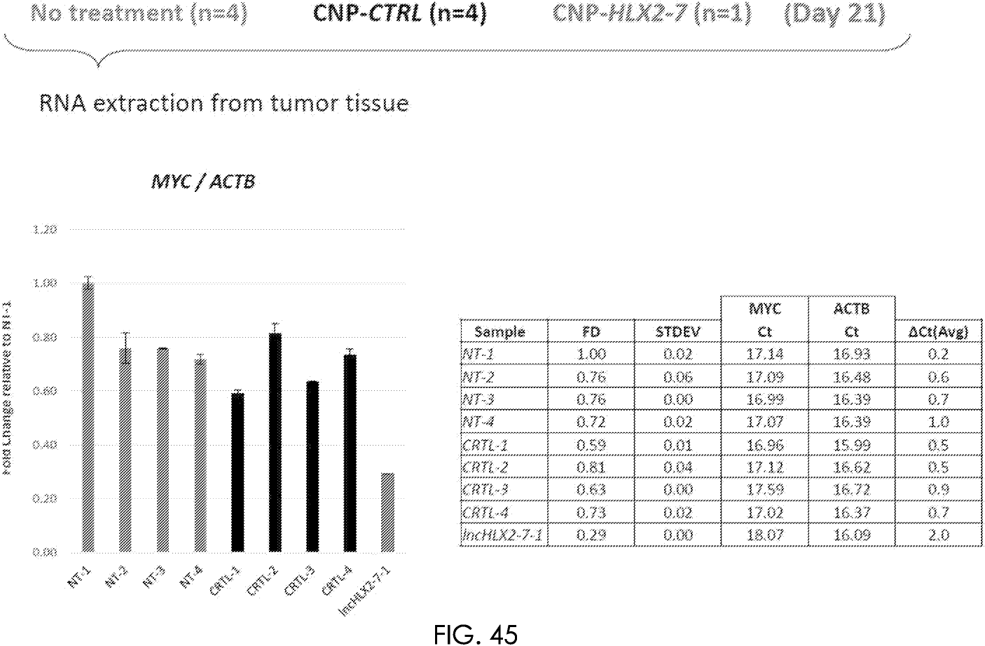

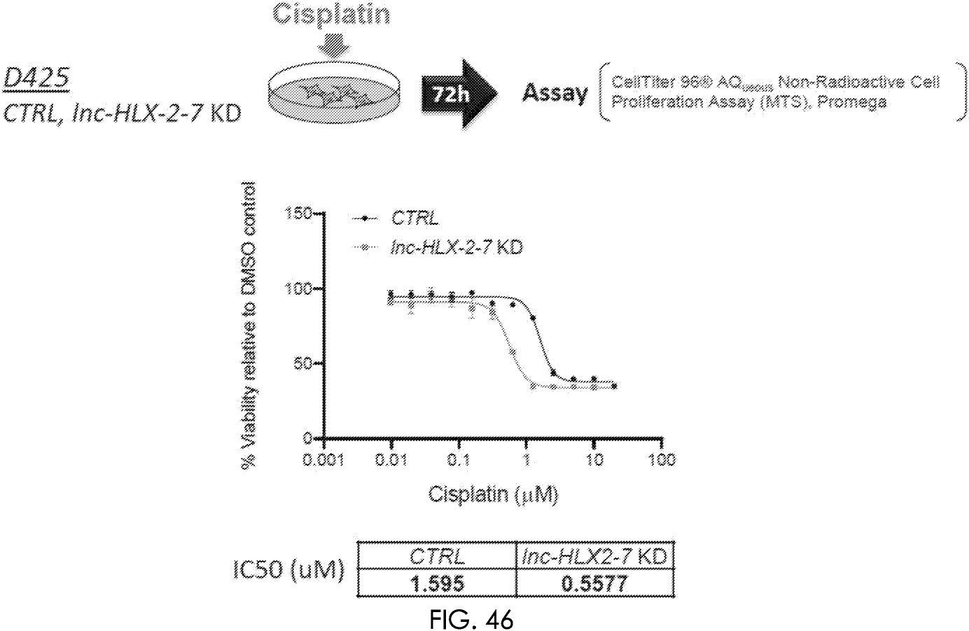

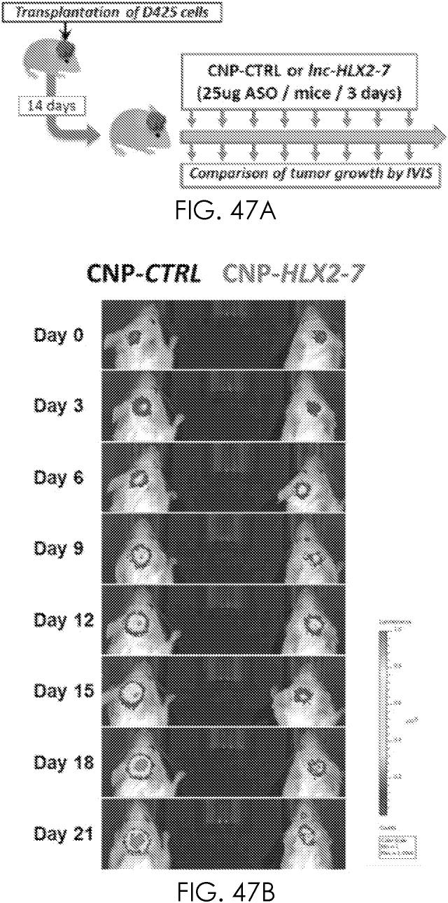

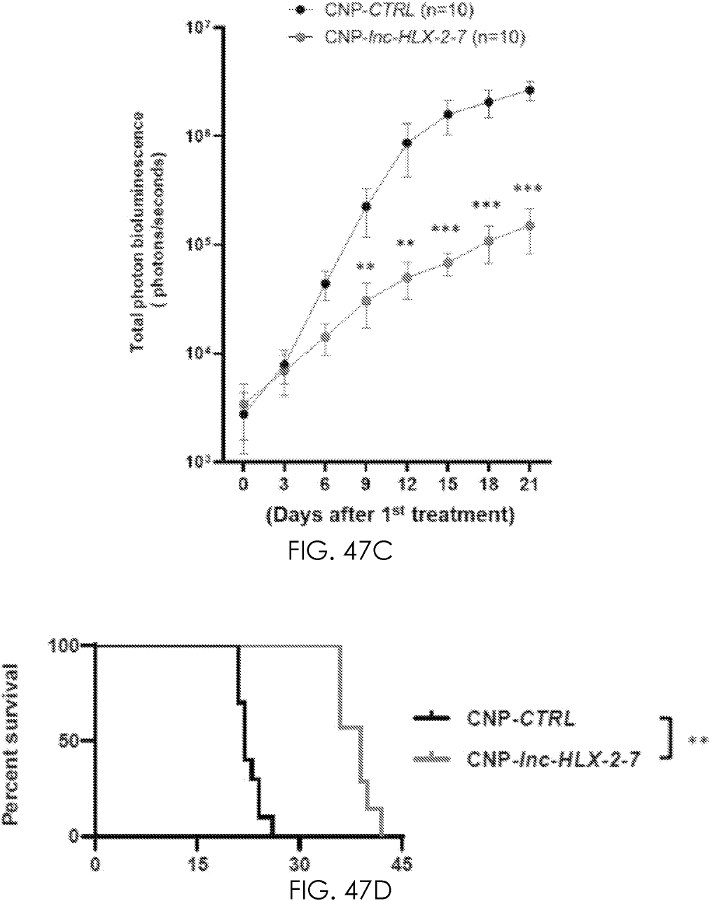

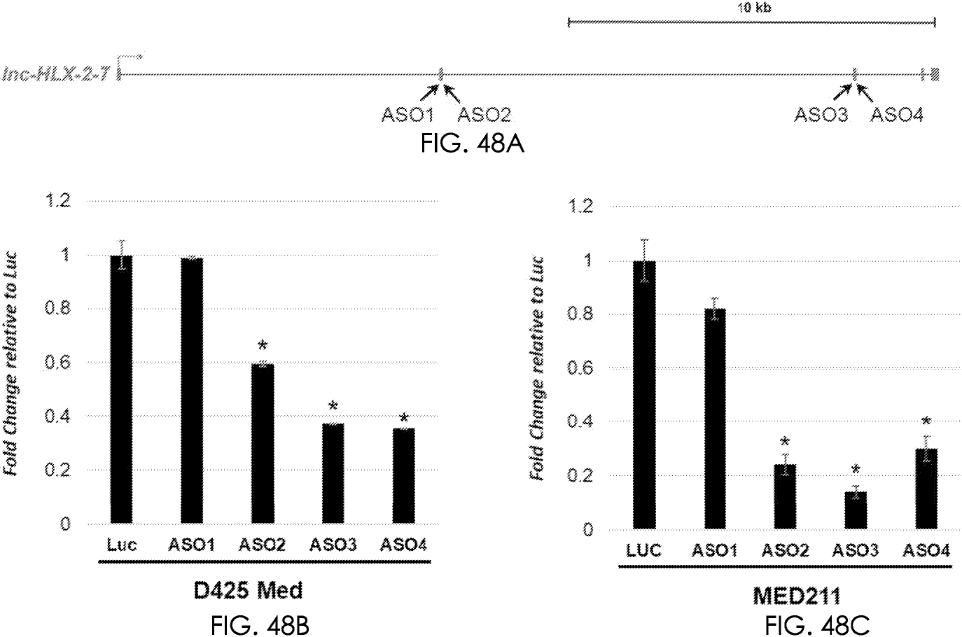

The compositions and methods of the present invention also comprise an ASO in association with an appropriate drug delivery system. In particular embodiments a polymeric micelle containing antisense oligonucleotides targeting lnc-HLX-2-7(ASO-HLX-2-7) is provided. In more particular embodiments, the polymeric micelle comprises cerium oxide nanoparticle (CNP). See FIGS. 40-41. In particular embodiments, the present invention provides methods comprising creating mixed valence state of cerium oxide nanoparticle for ASO conjugation. Such methods include, for example, controlling +3/+4 ratio for ASO- and related conjugation. In particular embodiments, the surface charge of the cerium nanoparticles are modified to encapsulate the polymeric micelle. In other embodiments, the surface charge of ASO-conjugated cerium oxide nanoparticles are modified to encapsulate the polymeric micelle. In certain embodiments, it is understood that as the nucleotide sequence of the ASO changes, then the cerium oxide nanoparticle surface is also modified.

Accordingly, in another aspect, the present invention provides methods and compositions useful for detecting long non-coding (lnc) RNAs. The methods for detection described herein can further comprise a treatment step. In one embodiment, the present invention provides a method comprising detecting lnc RNA HLX-2-7 in a biological sample obtained from a patient having or suspected of having medulloblastoma. In certain embodiments, the detecting step is performed using RNA fluorescence in situ hybridization (FISH) assay. In specific embodiments, the biological sample is a tissue sample. In particular embodiments, the tissue sample is a formalin-fixed paraffin-embedded (FFPE) sample. In a specific embodiment, the FISH assay comprises oligonucleotide probes that hybridize to lncHLX-2-7 (SEQ ID NO:200) and branched DNA signal amplification. In a more specific embodiment, the probes comprise at least one of SEQ ID NOS:3-4 and 8-21. In an alternative embodiment, the probes comprise SEQ ID NOS:3-4 and 8-21. In another embodiment, the probes further comprise at least one of SEQ ID NOS:5-7. In yet another embodiment, the probes further comprise SEQ ID NOS:5-7.

In another embodiment, the method further comprises detecting MYC expression in the biological sample. In specific embodiments, the biological sample is a tissue sample. In particular embodiments, the tissue sample is a formalin-fixed paraffin-embedded (FFPE) sample. In a specific embodiment, the FISH assay comprises oligonucleotide probes that hybridize to MYC (SEQ ID NO:202) and branched DNA signal amplification. In a more specific embodiment, the probes comprise at least one of SEQ ID NOS:51-56, 59-60, 62-63, 66-69, 72-73, 75-78, 81-98, 101-102. In a range of ‘n’ probes where ‘n’ is the total number of listed probes, the term “at least one of” includes the terms at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 ... up to and including n probes.

In an alternative embodiment, the probes comprise SEQ ID NOS:51-56, 59-60, 62-63, 66-69, 72-73, 75-78, 81-98, 101-102. In another embodiment, the probes further comprise at least one of SEQ ID NOS:57-58, 61, 64-65, 70-71, 74, 79-80, 99-100. In yet another embodiment, the probes further comprise SEQ ID NOS:57-58, 61, 64-65, 70-71, 74, 79-80, 99-100.

In embodiments detecting HLX-2-7 and/or MYC expression, the method can further comprise detecting lnc RNA SPRY4-IT1 in the biological sample. In specific embodiments, the biological sample is a tissue sample. In particular embodiments, the tissue sample is a formalin-fixed paraffin-embedded (FFPE) sample. In a specific embodiment, the FISH assay comprises oligonucleotide probes that hybridize to SPRY4-IT1 (SEQ ID NO:201) and branched DNA signal amplification. In a more specific embodiment, the probes comprise at least one of SEQ ID NOS:22-25 and 27-50. In an alternative embodiment, the probes comprise SEQ ID NOS:22-25 and 27-50. In another embodiment, the probes further comprise SEQ ID NO:26.

In embodiments detecting HLX-2-7, MYC and/or SPRY4-IT1 expression, the method can further comprise detecting MYCN in the biological sample. In specific embodiments, the biological sample is a tissue sample. In particular embodiments, the tissue sample is a formalin-fixed paraffin-embedded (FFPE) sample. In a specific embodiment, the FISH assay comprises oligonucleotide probes that hybridize to MYCN (SEQ ID NO:203) and branched DNA signal amplification. In a more specific embodiment, the probes comprise at least one of SEQ ID NOS:103-104, 107-108, 111-112, 114-125, 127-130, 133-144, 147-152. In an alternative embodiment, the probes comprise SEQ ID NOS:103-104, 107-108, 111-112, 114-125, 127-130, 133-144, 147-152. In another embodiment, the probes further comprise at least one of SEQ ID NOS:105-106, 109-110, 113, 126, 131-132, 145-146. In yet another embodiment, the probes further comprise SEQ ID NOS:105-106, 109-110, 113, 126, 131-132, 145-146.

In additional embodiments, the method can further comprise one or more lnc RNA selected from the group consisting of MIR100HG, USP2-AS1, lnc-CFAP100-4, ARHGEF7-AS2, lnc-HLX-1, lnc-EXPH5-2, lnc-CH25H-2, and lnc-TDRP-3. Such lnc RNAs can be used to distinguish Group 3 MB from Group 4 MB.

The compositions and methods of the present invention can be used to differentiate Group 3 MB from Group 4 MB. As described herein, HLX-2-7 can be used to differentiate Group 3 MB from Group 4 MB. HLX-2-7 is a Group 3 specific lncRNA in MB. In other embodiments, HLX-2-7 and MYC can be used together as Group 3 MBs have a higher MYC oncogene expression compared to other MB groups.

In further embodiments, the compositions and methods of the present invention also utilize detection of SPRY4-IT1 (“SPRIGHTLY”) and/or MYCN. SPRY4-IT1 is highly expressed primarily in Group 4 MB as compared to other groups. MYCN is also useful as a negative control for Group 3, as expression of MYCN is seen in Group 4.

In another aspect, the present invention provides compositions and methods useful for classifying all MB subgroups. In one embodiment, an 11 lnc RNA panel comprising MIR100HG, lnc-CFAP100-4, ENSG00000279542, lnc-ABCE1-5, USP2-AS1, lnc-RPL12-4, OTX2-AS1, lnc-TBC1D16-3, ENSG00000230393, ENGSG00000260249, and lnc-CCL2-2 is detected. In another embodiment, a 14 lnc RNA panel comprising DPYSL4, HUNK, PDIA5, PYY, CACNA1A, RBM24, KIF26A, DISP3, GABRA5, COL25A1, TENM1, GAD1, ADAMTSL1, and FBXL7 is detected. In an alternative embodiment, a 9 lnc RNA panel comprising MIR100HG, lnc-CFAP100-4, ENSG00000279542, lnc-ABCE1-5, USP2-AS1, lnc-RPL12-4, OTX2-AS1, lnc-TBC1D16-3, and ENSG00000230393 is detected.

In a further aspect, the present invention provides compositions and methods useful for prognosing patients having MB. In one embodiment, a 17 lnc RNA panel comprising lnc-TMEM258-3, ZNRF3-AS1, lnc-TMEM121-3, MAP3K14-AS1, LINC01152, KLF3-AS1, lnc-PRR34-1, lnc-FOXD4L5-25, AC209154.1, TTC28-AS1, FAM222A-AS1, LINC00336, LINC-01551, H19, lnc-RRM2-3, lnc-CDYL-1, and AL139393.2 is detected. See Table 6 which includes favorable prognosis markers and less favorable prognostic markers.

It is understood that in the embodiments in which a panel of lnc RNAs is detected, that the scope of such embodiments includes at least one of the recited panel. In a range of ‘n’ lnc RNAs where ‘n’ is the total number of listed lnc RNAs, the term “at least one of” includes the terms at least 1, at least 2, at least 3, at least 4, at least 5, at least 6, at least 7, at least 8, at least 9, at least 10, at least 11, at least 12, at least 13, at least 14, at least 15, at least 16 ... up to and including at least n lnc RNAs. For example, it is understood that in the 11 lnc RNA panel useful for classifying all MB subgroups, one can utilize at least one of the 11 lnc RNAs and such embodiments include at least 1, at least 2, at least 3, at least 4, at least 5, at least 6, at least 7, at least 8, at least 9, at least 10 and 11 lnc RNAs.

In particular embodiments, the methods of the present invention utilize a FISH assay. In such embodiments, the assay utilizes probe sets for the target RNA and branched DNA signal amplification. For example, a probe set of oligonucleotide pairs hybridizes to the target RNA. Signal amplification is achieve through hybridization of adjacent oligonucleotide pairs to bDNA structured, which are formed by pre-amplifiers, amplifiers and fluorochrome-conjugated label probes. These embodiments result in greater specificity, lower background and higher signal-to-noise ratios. The probes useful for detection of HLX-2-7, MYC, SPRY4-IT1, MYCN and MALAT1 (a control) are shown in Tables 1-5, respectively. Such oligos include label extenders and blocker oligos.

In another aspect, the present invention provides compositions and methods useful for detecting HLX-2-7, as well as SPRY4-IT1, MYC and/or MYCN in cerebrospinal fluid. In such embodiments, the targets are detected in CSF using polymerase chain reaction including, but not limited to, qPCR and digital PCR.

In yet another aspect, the present invention provides methods of treatment. Such methods can include the detection of HLX-2-7, as well as SPRY4-IT1, MYC and/or MYCN, followed by treatment of the patient. Further embodiments include detection of at least one of MIR100HG, USP2-AS1, lnc-CFAP100-4, ARHGEF7-AS2, lnc-HLX-1, lnc-EXPH5-2, lnc-CH25H-2, and lnc-TDRP-3. In still further embodiments, detection can include the lnc RNA panels also described herein. Treatment can include maximal safe surgical resection, radiotherapy and chemotherapy (e.g., cisplatin, cyclophosphamide, vincristine, lomustine, in various dosing regimens; standard dosing is typically 9 cycles, high dose is typically 4 cycles). Combination treatment can be used including, but not limited to, pemetrexed and gemcitabine. Surgery may be needed to treat hydrocephalus (fluid build-up in the skull) and to remove the tumor. Treatment can further include (alone or in combination) endoscopic third ventirculostomy (ETV) or ventriculo-peritoneal shunt (VP shunt). Indeed, the markers described herein can be used to decide whether to reduce radiation, provide a prognosis and reduce chemo exposure

In another aspect, lnc-HLX-2-7can be used as a target for therapy. In certain embodiments, expression of lnc-HLX-2-7can be disrupted. Knock out technology can comprise gene editing. For example, gene editing can be performed using a nuclease, including CRISPR associated proteins (Cas proteins, e.g., Cas9), Zinc finger nuclease (ZFN), Transcription Activator-Like Effector Nuclease (TALEN), and meganucleases. In other embodiments, expression of the target can be disrupted using RNA interference technology including, but not limited to, a short interfering RNA (siRNA) molecule, a microRNA (miRNA) molecule, or an antisense molecule.

In a further aspect, the present invention provides one or more probes useful in the methods described herein. In one embodiment, the probes bind HLX-2-7 and comprise at least one of SEQ ID NOS:3-21. In another embodiment, the probes bind SPRY4-IT and comprise at least one SEQ ID NOS:22-50.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Example 1: The long non-coding RNA lnc-HLX-2-7is oncogenic in group 3 medulloblastomas. Group 3 MBs are associated with poor clinical outcomes, are difficult to subtype clinically, and have a biology that is poorly understood. In an effort to address these problems, we identified a Group 3-specific long noncoding RNA, lnc-HLX-2-7, in an in silico analysis of 175 MBs and confirmed its expression in Group 3 MB cell lines, patient-derived xenografts, and formalin-fixed paraffin-embedded samples. Knockdown of lnc-HLX-2-7 significantly reduced cell growth and induced apoptosis. Deletion of lnc-HLX-2-7 in cells injected into mouse cerebellums reduced tumor growth compared with parental cells, and bulk and single-cell RNAseq of these tumors revealed modulation of cell viability, cell death, and energy metabolism signaling pathways. The MYC oncogene regulated lnc-HLX-2-7, and its expression was reduced by JQ1. Lnc-HLX-2-7 is a candidate biomarker and a potential therapeutic target in Group 3 MBs.

### Introduction

Medulloblastoma (MB) is the most common malignant pediatric brain tumor.1 Recent large-scale and high-throughput analyses have subclassified MBs into 4 molecularly distinct subgroups, each characterized by specific developmental origins, molecular features, and prognoses. ¹⁻⁴ The well-characterized WNT and SHH subgroups have been causally linked to activated wingless and sonic hedgehog developmental cascades, respectively. 1 However, significant gaps remain in our understanding of the signaling pathways underlying Group 3 and Group 4 MBs, which account for 60% of all diagnoses and are frequently metastatic at presentation (~40%).⁴ Group 3 and Group 4 tumors display significant clinical and genetic overlap, including similar location and presence of isochromosome 17q, and identifying these subgroups can be challenging without the application of multigene expression or methylation profiling. Therefore, improved understanding of Group 3 tumor drivers and theranostic targets is urgently needed.

The vast majority of the genome serves as a template for not only coding RNAs but also noncoding RNAs (ncRNAs). Of the noncoding RNAs, long noncoding RNAs (lncRNAs), which describe a class of RNAs >200 nucleotides in length, have been widely investigated and identified as key regulators of various biological processes, including cellular proliferation, differentiation, apoptosis, migration, and invasion.⁵⁻⁸ LncRNAs are functionally diverse and participate in transcriptional silencing,⁹ function as enhancers,¹⁰ and sequester miRNAs from their target sites.¹¹ LncRNAs can also act as hubs for protein-protein and protein-nucleic acid interactions.¹² There is now a considerable body of evidence implicating lncRNAs in both health and disease, not least human tumorigenesis.^{8,13,14} It has recently been reported that various lncRNAs play important roles in MB biology,^{2,15-18} although the functional significance of many remains uncertain. Since many lncRNAs are uniquely expressed in specific cancer types,¹⁹ they may function as powerful MB subgroup-specific biomarkers and therapeutic targets.

By analyzing RNA sequencing data derived from human MBs, here we report that the novel lncRNA lnc-HLX-2-7differentiates Group 3 from other MBs. Deletion by clustered regularly interspaced short palindromic repeat (CRISPR)/ CRISPR associated protein (CRISPR/Cas9) of lnc-HLX-2-7 in Group 3 MB cells significantly reduced cell growth in vitro and in vivo. RNA sequencing of xenografts revealed lnc-HLX-2-7-associated modulation of cell viability and cell death signaling pathways. Lnc-HLX-2-7 is a promising novel biomarker and potential therapeutic target for Group 3 MBs.

### Materials and Methods

MB Tissue and RNA Samples. Eighty MB tissue samples obtained from a tumor database maintained by the Department of Pathology at the Johns Hopkins Hospital were analyzed (Table 7) under institutional review board (IRB) approved protocol NA_00015113. Detailed information about the RNA samples are described in the Supplemental Materials and Methods.

Patient In Silico Data. Raw FASTQ files for RNA sequencing data corresponding to 175 MB patients (referred to as the ICGC dataset) belonging to the 4 MB subgroups (accession number EGAS00001000215) were downloaded from the European Genome-Phenome Archive (EGA, http://www.ebi.ac.uk/ega/) after obtaining IRB approval.²⁰

Cell Culture. Cell lines were authenticated using single tandem repeat profiling. D425 Med cells were cultured in DMEM/ F12 with 10% serum and 1% glutamate/penicillin/streptomycin. MED211 cells were cultured in medium composed of 30% Ham’s F12/70% DMEM, 1% antibiotic antimycotic, 20% B27 supplement, 5 µg/mL heparin, 20 ng/mL epidermal growth factor (EGF), and 20 ng/mL fibroblast growth factor 2. DAOY cells were cultured in DMEM with 10% serum and 1% glutamate/penicillin/streptomycin. All cells were grown in a humidified incubator at 37° C., 5% CO2. For blocking of bromodomain and extraterminal domain family (BET) bromodomain protein in D425 Med and MED211 cells, Jun Qi 1 (JQ1) (SML1524-5MG, Sigma Aldrich) was added, and the medium was changed every other day.

Quantitative Real-Time PCR. Total RNA was purified using the Direct-zol RNA Miniprep kit (Zymo Research). To obtain RNA from xenografts, tumor tissues were pulverized and then used for purification. Quantitative PCR was carried out using SYBR Green mRNA assays as previously described.⁸ Primer sequences are listed in Tables 8-9 and Supplementary Table 2 (available online).

Antisense Oligonucleotides. Lnc-HLX-2-7 Antisense oligonucleotides (ASOs) were designed using the Integrated DNA Technologies (IDT) Antisense Design Tool (IDT). ASO knockdowns were performed with 50 nM (final concentration) locked nucleic acid (LNA) GapmeRs transfected with Lipofectamine 3000 (Thermo Fisher Scientific). All ASOs were modified with phosphorothioate (PS) linkages.

The following ASOs were used: ASO targeting lnc-HLX-2-7(ASO-lnc-HLX-2-7): +T^{∗}+G^{∗}+A^{∗}G^{∗}A^{∗}G^{∗}A^{∗}T^{∗}T^{∗}A^{∗}A^{∗}T^{∗}C^{∗}T^{∗}A^{∗}G^{∗}A^{∗}T^{∗}+T^{∗}+G^{∗}+C (SEQ ID NO:240) and control ASO targeting luciferase (ASO-Luc): +T*+C*+G*A*A*G*T*A*C*T*C*A*G*C*G*T*A*A*+G*+T*+T (SEQ ID NO:241). The PS linkages are indicated with * and LNA modified oligonucleotides are indicated with +. Other ASOs are described herein.

SiRNA-Mediated Knockdown of HLX, MYC and MYCN. Small interfering (si)RNAs targeting HLX (catalog no. 4427037, ID: s6639) and MYC (catalog no. 4427037, ID: s9129) were purchased from Thermo Fisher Scientific. SiRNAs were transfected at 20 nM for 48 h using Lipofectamine RNAiMAX (Thermo Fisher Scientific). The efficiency was determined by quantitative real-time (qRT)-PCR.

Cell Proliferation, Apoptosis, and 3D Colony Formation Assays. Cells were plated in 96-well plates at 5 × 103 cells per well in triplicate. After 72 hours of ASO or siRNA transfection, living cells were counted by trypan blue staining. Apoptotic cells were analyzed using a GloMax luminometer (Promega) with conditions optimized for the Caspase-Glo 3/7 Assay. For the 3D colony formation assay, cells were seeded in 24-well plates at a density of 1 × 102 cells/well and were stained with crystal violet solution approximately 14 days later. Colony number was determined using the EVE cell counter (Nano Entek), and staining intensity was analyzed using ImageJ software.

Lnc-HLX7 CRISPR/Cas9 Knockdown in D425 Med Cells. The single guide RNA (sgRNA) targeting lnc-HLX7 was designed using Zhang Lab resources (http://crispr. mit.edu/) and synthesized to make the lenti-lnc-HLX- 2-7-sgRNA-Cas9 constructs as described previously.²¹

The DNA sequences for generating sgRNA were forward: 5′- GGACCCACTCTCCAACGCAG -3′ (SEQ ID NO:1) and reverse: 5′- GCAGGGACCCCTCATTGACG -3′ (SEQ ID NO:2). For the control plasmid, no sgRNA sequence was inserted into the construct. Lnc-HLX-2-7-edited cells and control cells were selected using 4 µg/mL puromycin. To determine the genome editing effect, total RNA was extracted from the lnc- HLX-2-7-edited cells and control cells and the expression of lnc-HLX-2-7 quantified by qRT-PCR.

Medulloblastoma Xenografts (Intracranial). All mouse studies were approved and performed in accordance with the policies and regulations of the Animal Care and Use Committee of Johns Hopkins University. Intracranial MB xenografts were established by injecting D425 Med cells, MED211 cells, D425 Med cells with lnc- HLX-2-7 deleted, and MED211 cells with lnc-HLX-2-7 deleted into the cerebellums of NOD-SCID mice (Jackson Laboratory). Cerebellar coordinates were -2 mm from lambda, +1 mm laterally, and 1.5 mm deep. Seven days after injection, mice were administered JQ1 (50 mg/ kg) or vehicle alone (DMSO) on alternating days via intraperitoneal injection for 14 days. Tumor growth was evaluated by weekly bioluminescence imaging using an in vivo spectral imaging system (IVIS Lumina II, Xenogen).

Immunohistochemistry. For the analysis of cell proliferation, tumor sections were incubated with anti-Ki67 (Alexa Fluor 488 Conjugate) antibodies (#11882, 1:200, Cell Signaling Technology) at 4° C. overnight. For the analysis of apoptosis, DeadEnd Fluorometric TUNEL System (Promega) was performed on the tumor sections, according to the manufacturer’s instructions. The stained sections were imaged using a confocal laser-scanning microscope (Nikon C1 confocal system; Nikon). The acquired images were processed using the NIS (Nikon) and analyzed with ImageJ software (https://imagej.nih.gov/ij/).

Chromatin Immunoprecipitation. Cells (1 × 106) were treated with 1% formaldehyde for 8 minutes to crosslink histones to DNA. The cell pellets were resuspended in lysis buffer (1% sodium dodecyl sulfate, 10 mmol/L EDTA, 50 mmol/L Tris-HC1 pH 8.1, and protease inhibitor) and sonicated using a Covaris S220 system. After diluting the cell lysate 1:10 with dilution buffer (1% Triton-X, 2 mmol/L EDTA, 150 mmol/L NaCl, 20 mmol/L Tris-HC1 pH 8.1), diluted cell lysates were incubated for 16 h at 4° C. with Dynabeads Protein G (100-03D, Thermo Fisher Scientific) precoated with 5 µL of anti-MYC antibody (ab32, Abcam). Chromatin immunoprecipitation (ChIP) products were analyzed by SYBR Green ChIP-qPCR using the primers listed in Table 9.

RNA Library Construction and Sequencing. Total RNA was prepared from cell lines and orthotopic xenografts using Direct-zol RNA Miniprep kits (Zymo Research). RNA quality was determined with the Agilent 2100 Bioanalyzer Nano Assay (Agilent Technologies). Using a TruSeq Stranded Total RNA library preparation Gold kit (Illumina), strand-specific RNA-seq libraries were constructed as per the instructions. The quantification and quality of final libraries were determined using KAPA PCR (Kapa Biosystems) and a high-sensitivity DNA chip (Agilent Technologies), respectively. Libraries were sequenced on an Illumina NovaSeq 6000 using 1 × 50 base paired-end reads. Detailed methods of sequence and data analysis are described in Supplemental Materials and Methods.

Ingenuity Pathway Analysis. To analyze pathways affected by lnc-HLX-2-7, differentially expressed genes between D425 Med and D425 Med with lnc-HLX-2-7 deleted were compiled and analyzed using Qiagen Ingenuity Pathway Analysis (IPA). Analysis was conducted via the IPA web portal (www.ingenuity.com).

Data Availability. RNA-seq data described in the manuscript are accessible at NCBI GEO accession number GSE151810 and GSE156043.

RNA Fluorescence In Situ Hybridization. RNA was visualized in paraffin-embedded tissue sections using the QuantiGene ViewRNA ISH Tissue Assay Kit (Affymetrix). In brief, tissue sections were rehydrated and incubated with proteinase K. Subsequently, they were incubated with ViewRNA probesets designed against human lnc-HLX-2-7, MYC, and MYCN (Affymetrix). Custom type 1 primary probes targeting lnc-HLX-2-7, type 6 primary probes targeting MYC, and type 6 primary probes targeting MYCN were designed and synthesized by Affymetrix (Supplementary Table 2 (available online)). Hybridization was performed according to the manufacturer’s instructions.

Statistical Analysis. Statistical analyses were performed using GraphPad Prism software and the Limma R package. Data are presented as mean ± SD of 3 independent experiments. Differences between 2 groups were analyzed by the paired Student’s t-test and correlations with the Pearson correlation coefficient. Kruskal-Wallis analysis was used to evaluate the differences between more than 2 groups. Survival analysis was performed using the Kaplan-Meier method and compared using the log-rank test.

### Results

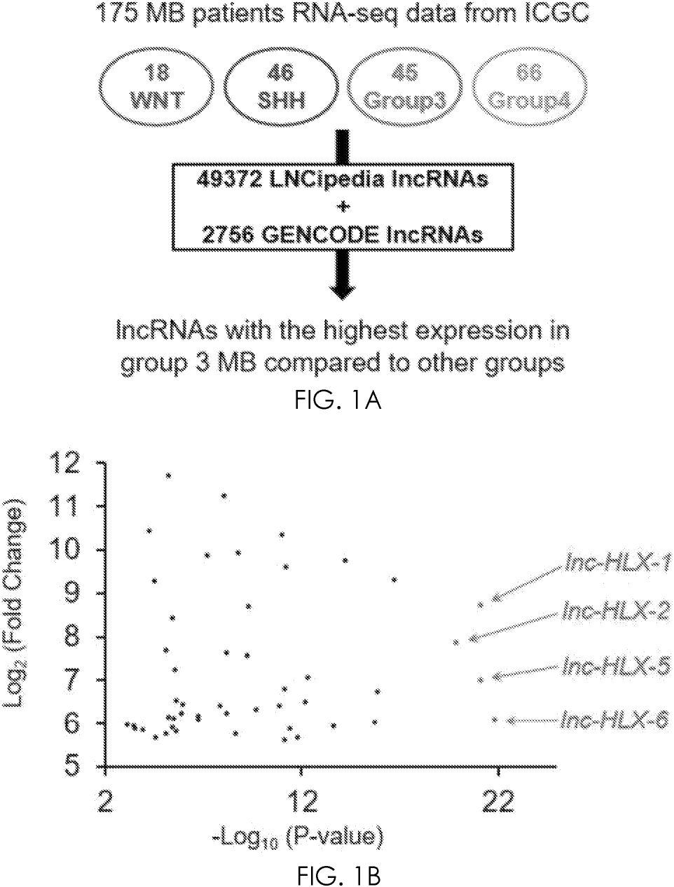

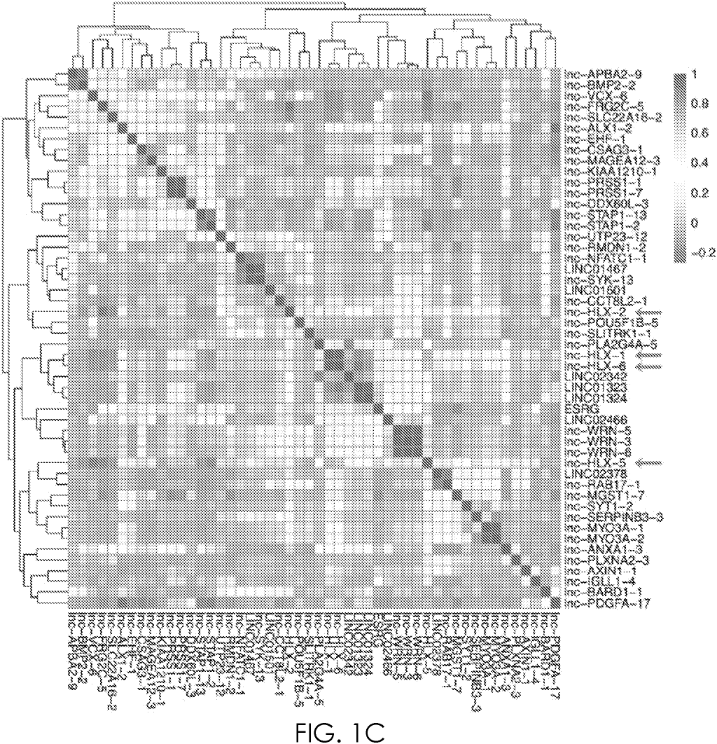

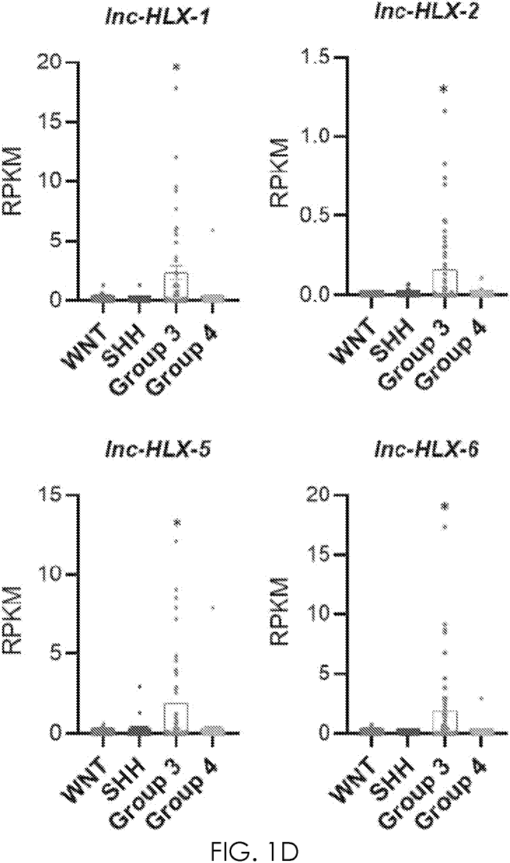

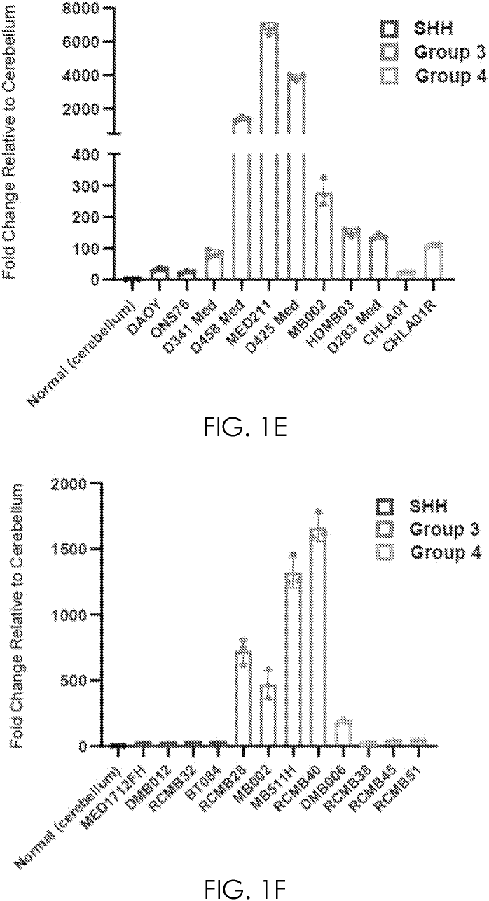

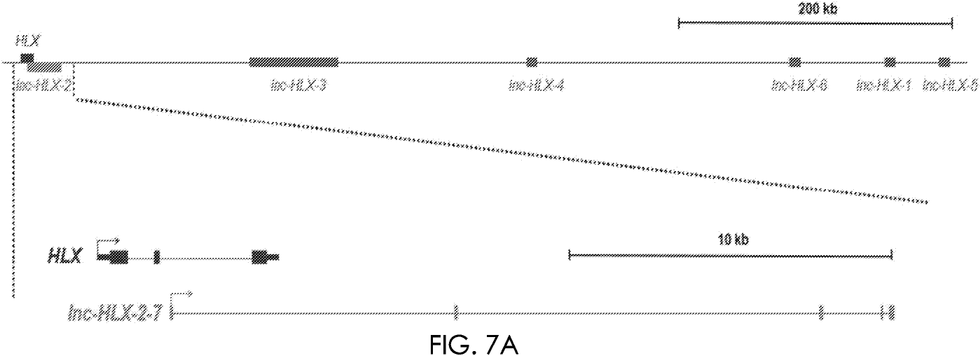

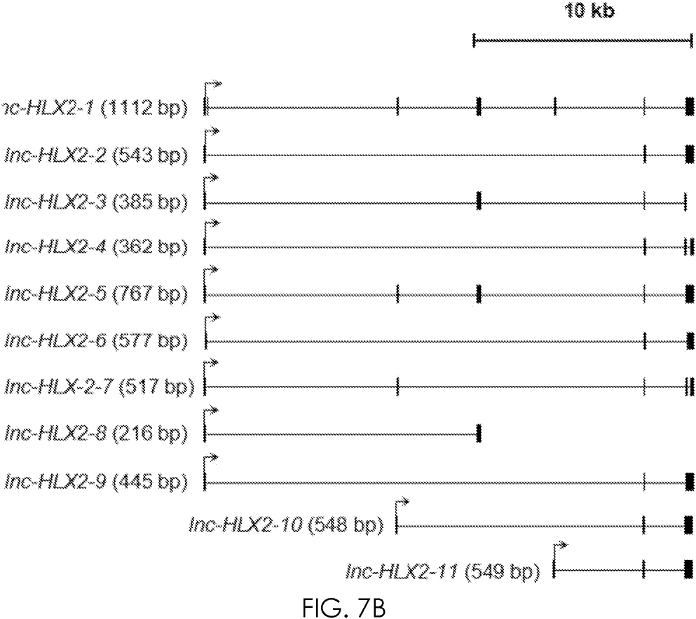

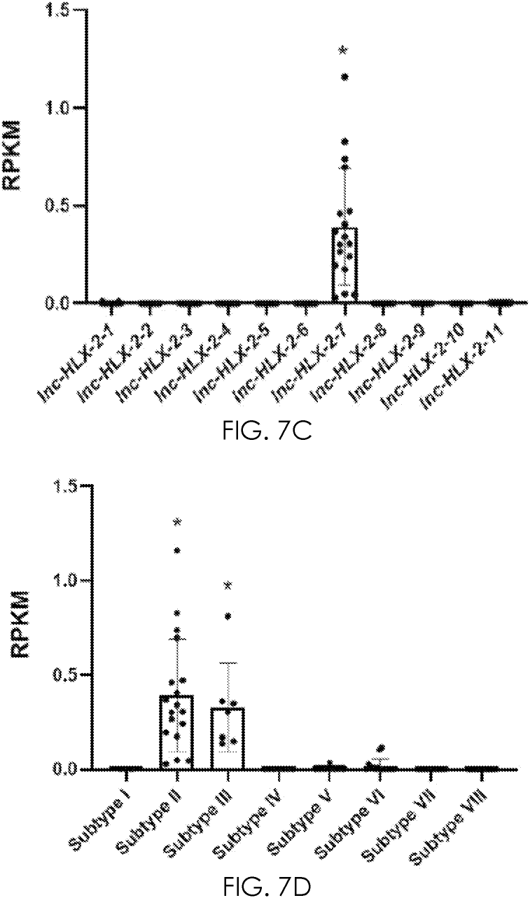

Identification of the Group 3-Specific Long-Noncoding RNA, lnc-HLX-2-7. To identify MB Group 3-specific lncRNAs, we obtained 175 RNA-seq files (FASTq) representing the 4 MB subgroups (WNT, SHH, Group 3, and Group 4) from the EGA and applied combined GENCODE and LNCipedia annotations.²² Given the need to find novel biomarkers that differentiate Group 3 from other groups, we identified a set of lncRNAs (lnc-HLX-1, lnc-HLX-2, Inc-HLX-5, and Inc-HLX-6) with markedly elevated and significant overexpression in Group 3 MB (FIGS. 1A, 1B and Table 10). Lnc- HLX-1, Inc-HLX-2, lnc-HLX-5, and lnc-HLX-6 showed a high expression correlation (FIG. 1C) and were highly expressed in Group 3 MB patient samples compared with other subgroups (P < 0.01; FIG. 1D). We recently reported that some of these lncRNAs also show Group 3-specific differential expression.²³ Due to lnc-HLX-2′s proximity to its host coding gene transcription factor and homeobox gene HB24 (HLX) and a recent study reporting that the lnc-HLX-2 region is a Group 3 MB-specific enhancer region (Supplementary FIGS. 1 (available online)),²⁴ we focused on lnc-HLX-2. Lnc-HLX-2 is located 2300 bp downstream of the transcriptional start site (TSS) of HLX (FIG. 7A) and consists of 11 transcripts (lnc-HLX-2-1 to Inc-HLX-2-11; FIG. 7B), of which lnc-HLX-2-7was highly expressed in Group 3 MBs (FIG. 7C). Quantitative RT-PCR analysis verified that lnc-HLX-2-7was highly upregulated in Group 3 MB cell lines (FIG. 1E) and patient-derived xenograft (PDX) samples (FIG. 1F) compared with other groups. It was recently shown through a combined analysis of Group 3 and 4 MBs that they can be further subdivided into 8 molecular subtypes, designated I to VIII.²⁰ In a combined analysis of Group 3 and Group 4 cases, lnc-HLX-2-7showed high expression in subtype II and III MBs compared with other subtypes (FIG. 7D).

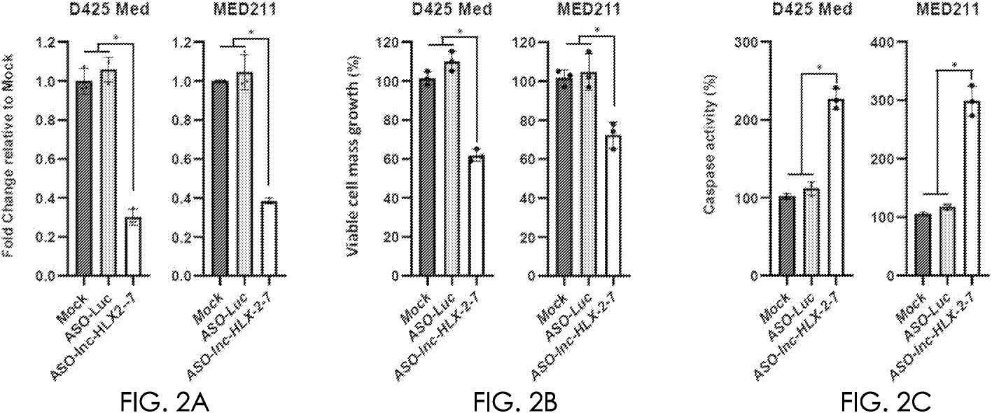

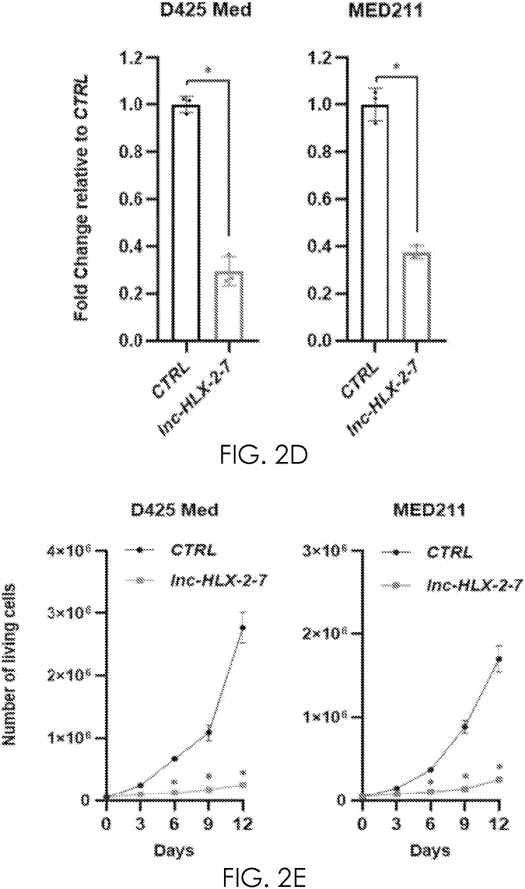

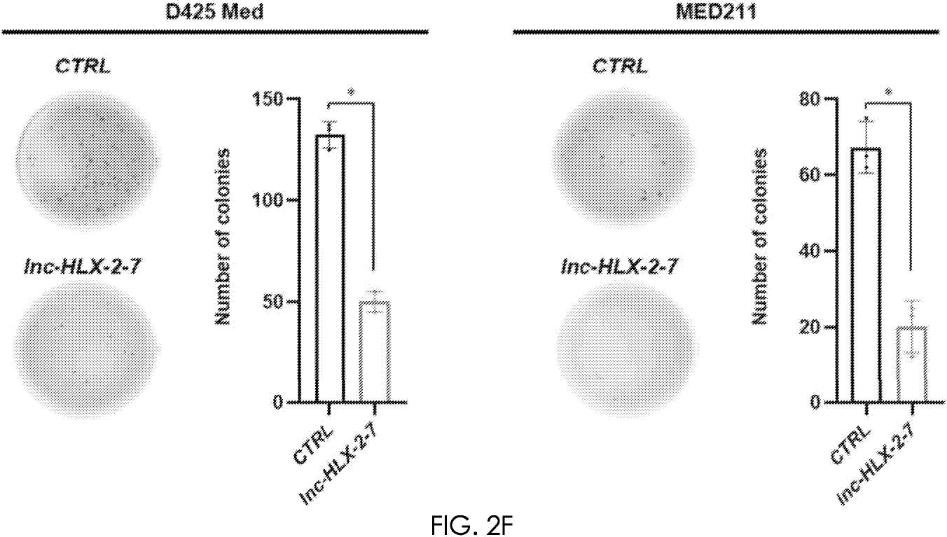

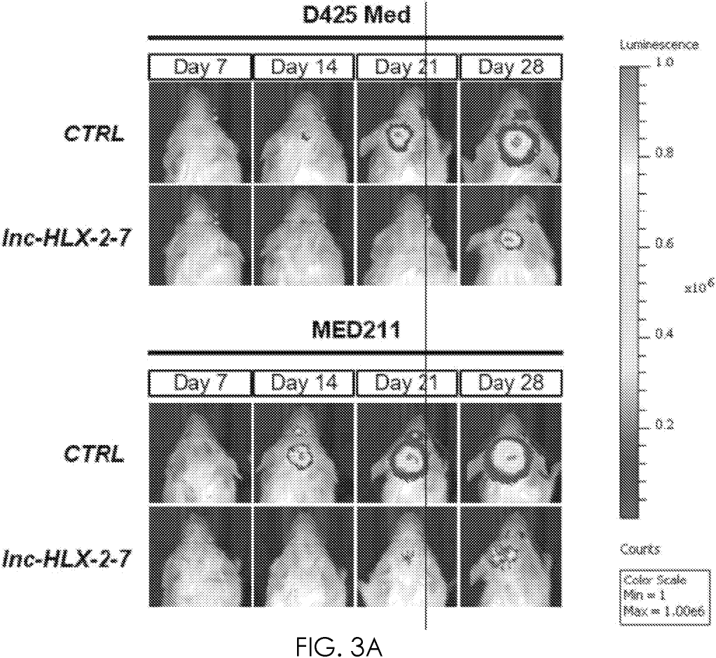

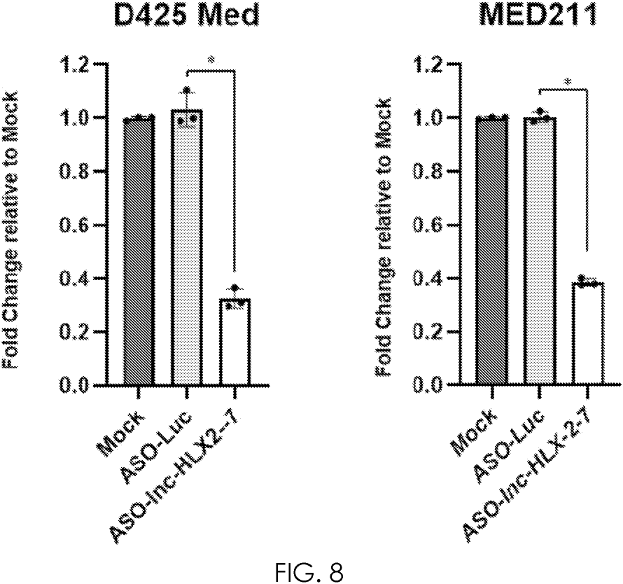

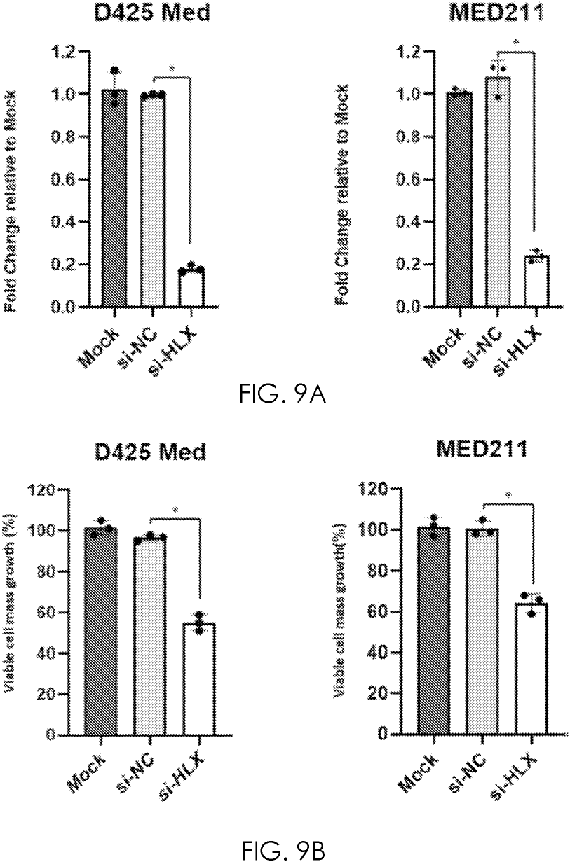

Lnc-HLX7 Functions as an Oncogene In Vitro. To investigate the function of lnc-HLX7, we used ASOs to inhibit lnc-HLX7 expression in D425 Med and MED211 MB cells. Transfection with ASO-lnc-HLX-2-7 significantly decreased lnc-HLX-2-7 expression compared with controls (ASO-Luc) in both cell lines (P < 0.01; FIG. 2A), which significantly suppressed MB cell growth and induced apoptosis (P < 0.01; FIGS. 2B, C). Next, CRISPR/Cas9 knock-down was used to generate single-cell colonies and further investigate the effect of lnc-HLX-2-7 in MB cells. We generated stable D425 Med and MED211-lnc-HLX-2-7-sgRNA cells, which constitutively expressed sgRNAs against lnc-HLX-2-7 to reduce lnc-HLX-2-7 expression (FIG. 2D). As expected, D425 Med and MED211-lnc-HLX-2-7-sgRNA cells showed reduced growth (FIG. 2E) and colony-forming ability (FIG. 2F) compared with D425 Med and MED211 control cells in vitro. While the functions of the majority of lncRNAs are not yet known, some have been shown to function in cis by regulating the expression of neighboring genes.²⁵⁻²⁷ Since lnc-HLX-2-7 is located downstream of the HLX TSS (FIG. 7A), we determined whether lnc-HLX-2-7 regulates HLX expression; indeed, HLX expression was significantly reduced in D425 Med and MED211 cells following treatment with ASO-lnc-HLX-2-7 (FIG. 8). In addition, HLXknockdown significantly decreased the growth of D425 Med and MED211 cells (FIGS. 9). While the current study focuses on the role of lncRNA HLX-2-7, understanding the molecular function of its hostcoding gene HLX requires further investigation, which is ongoing.

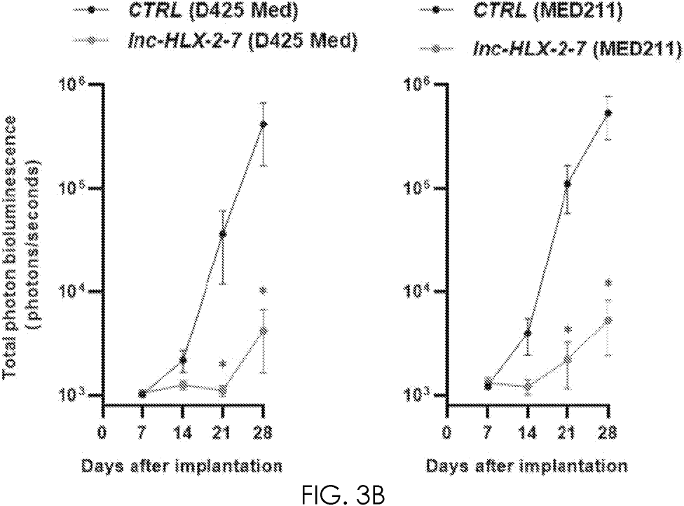

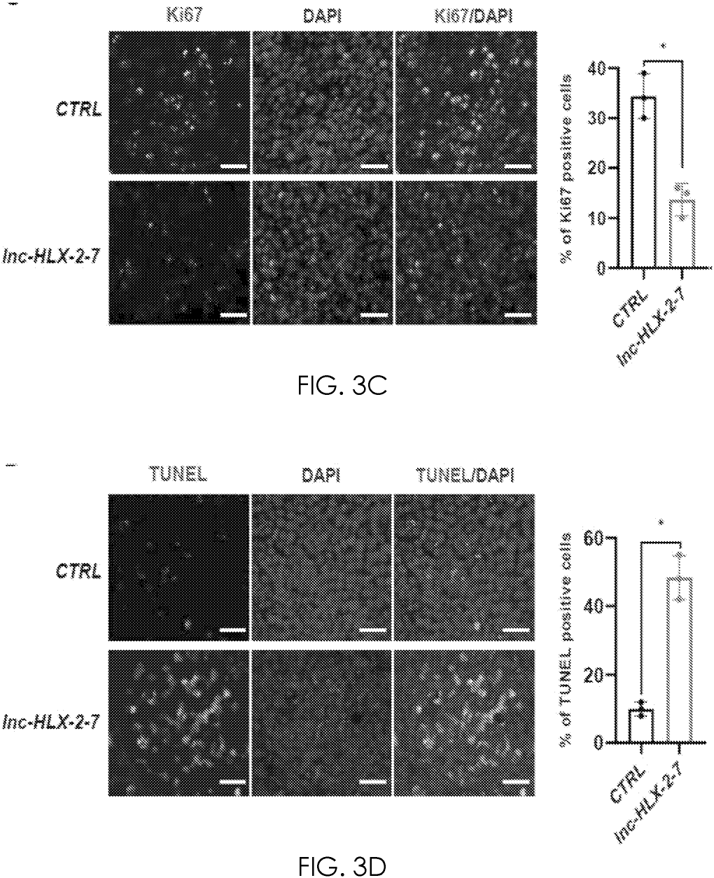

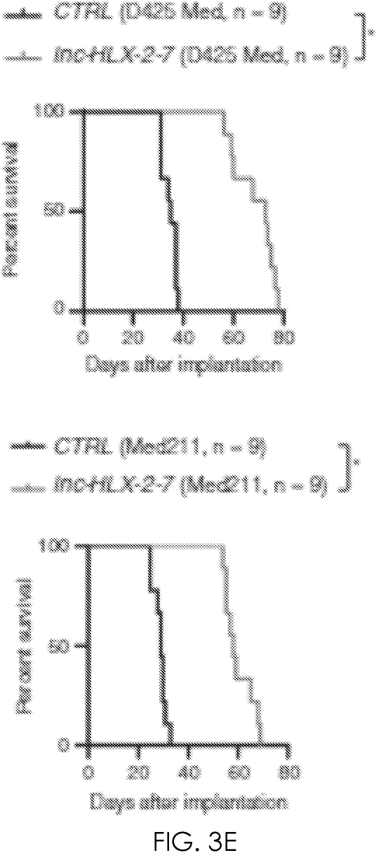

Lnc-HLX7 Regulates Tumor Formation in Mouse Intracranial Xenografts. To evaluate the effect of lnc-HLX7 on tumor growth in vivo, we established intracranial MB xenografts in NODSCID mice. D425 Med and MED211 control cells and D425 Med and MED211-lnc-HLX7-sgRNA cells were preinfected with a lentivirus containing a luciferase reporter. Weekly evaluation of tumor growth by bioluminescence imaging revealed significantly smaller tumors in mice transplanted with D425 Med and MED211-lnc-HLX7-sgRNA cells compared with mice transplanted with control cells (n = 9, P < 0.05; FIGS. 3A, 3B). At day 30, tumors were harvested and cut into sections and then subjected to Ki67 and TUNEL staining. Ki67 analysis showed reduced cell proliferation in D425 Med-lnc-HLX-2-7-sgRNA cell-transplanted mice (P < 0.01; FIG. 3C). TUNEL analysis found out that lnc-HLX-2-7 depletion induced significantly higher percentage of TUNEL-positive cells than compared with mice transplanted with control cells (P < 0.01; FIG. 3D). Kaplan-Meier plots demonstrated that the group transplanted with D425 Med and MED211-lnc-HLX-2-7-sgRNA cells had significantly prolonged survival compared with the control (FIG. 3E). Together, these results demonstrate that lnc-HLX-2-7regulates tumor growth in vivo and may function as an oncogene.

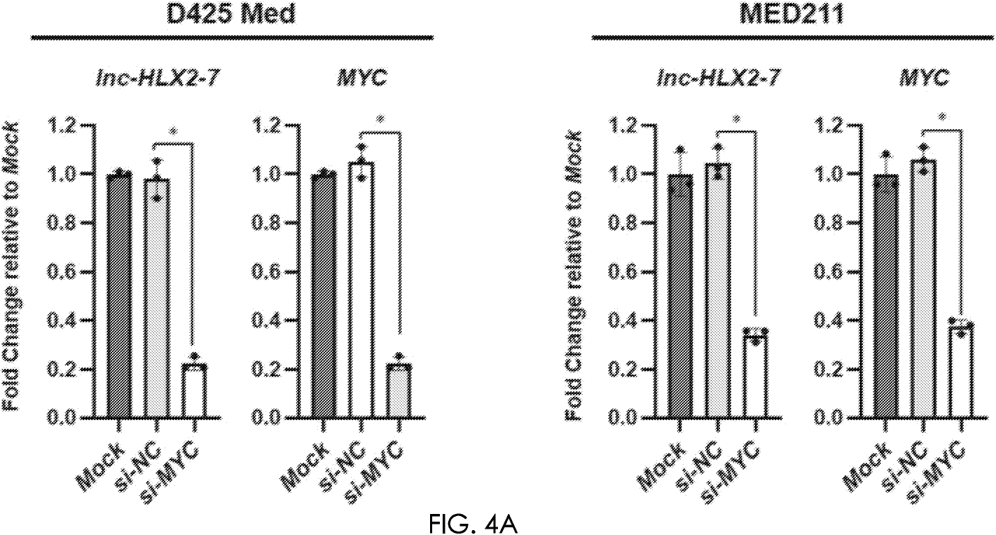

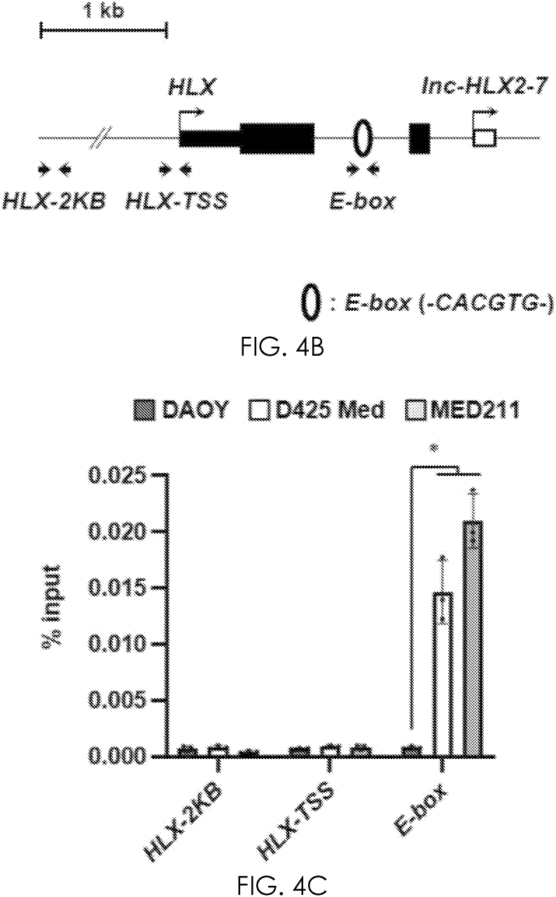

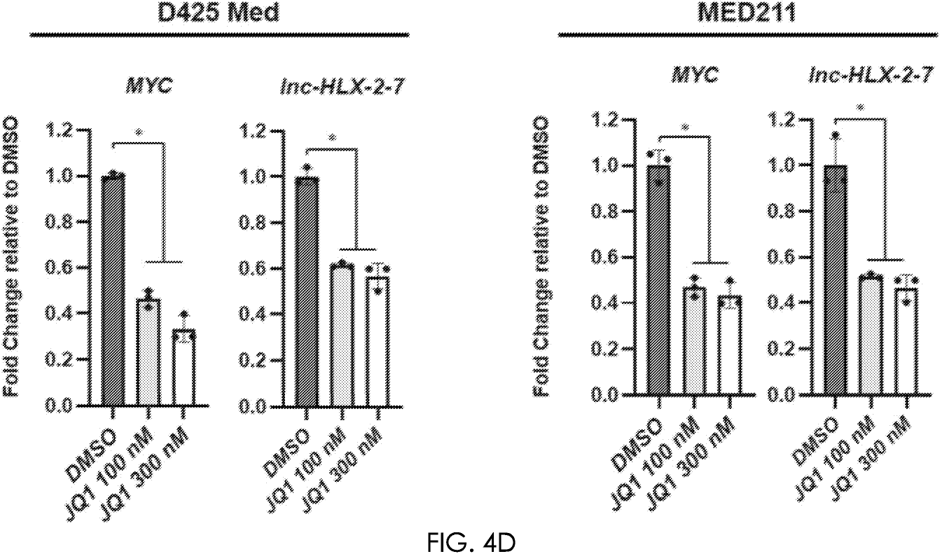

Transcriptional Regulation of lnc-HLX-2-7by the MYC Oncogene. Since the majority of Group 3 tumors exhibit elevated expression and amplification of the MYC oncogene,^{2,28} we hypothesized that MYC may regulate the expression of lnc-HLX-2-7. We therefore knocked down MYC by siRNA in D425 Med and MED211 cells, which decreased the expression of both MYC and lnc-HLX-2-7(FIG. 4A), suggesting that MYC may be an upstream regulator of lnc-HLX-2-7. To further support this, we also identified a MYC-binding motif (E-box; -CACGTG-) 772 bp upstream of the putative TSS of lnc-HLX-2-7 using the JASPAR CORE database (http://jaspar.genereg.net/)²⁹ (FIG. 4B). To test whether MYC could interact with the endogenous lnc-HLX-2-7 promoter, ChIP was performed in D425 Med and MED211 cells. ChIP analysis revealed that MYC bound to the E-box motif within the upstream region of lnc-HLX-2-7 in D425 Med and MED211 cells, but not in DAOY cells (FIG. 4C). These results strongly suggest that MYC is a direct regulator of lnc-HLX-2-7.

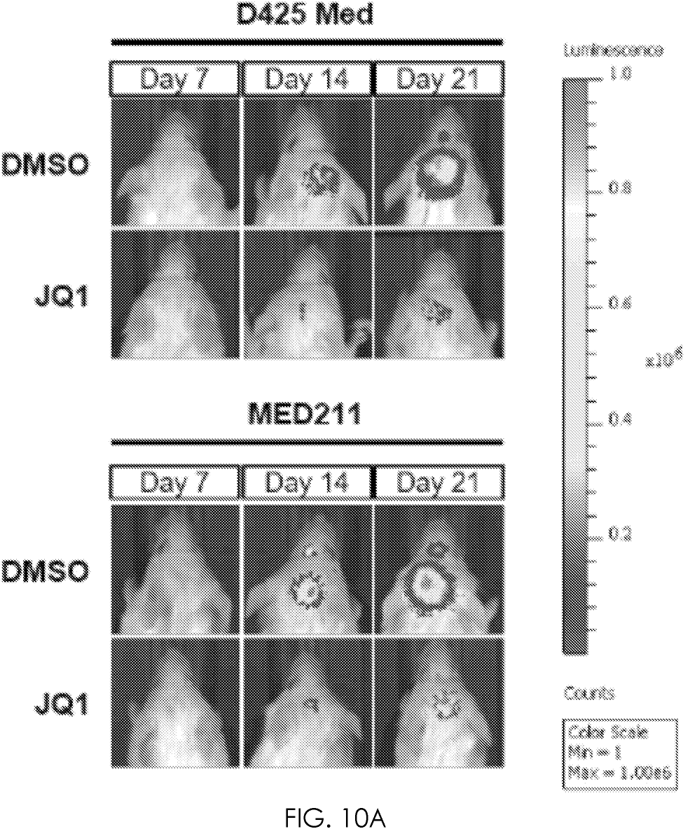

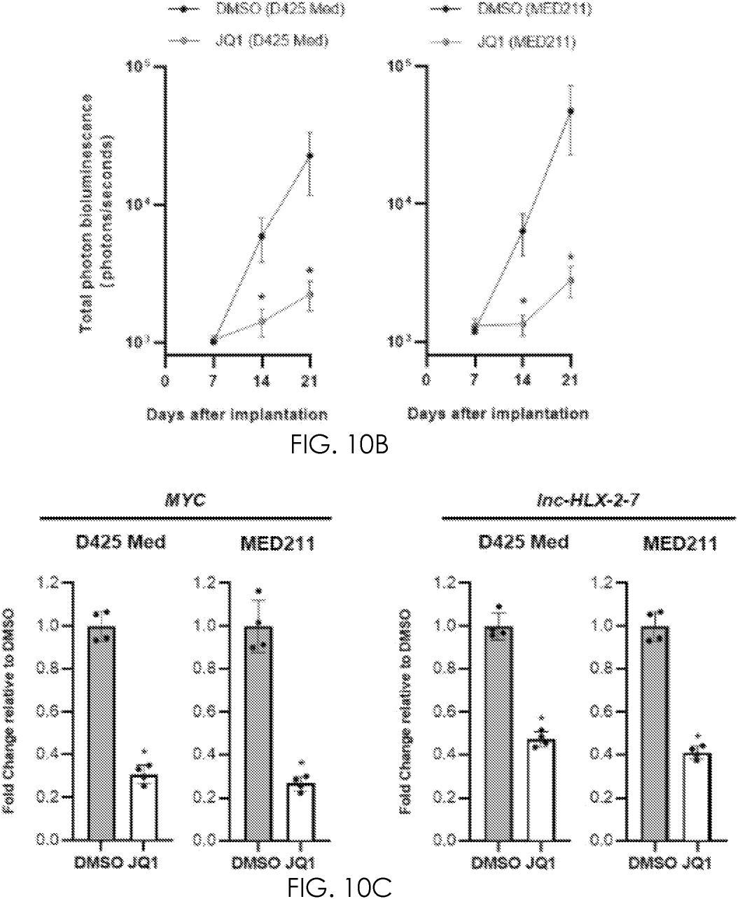

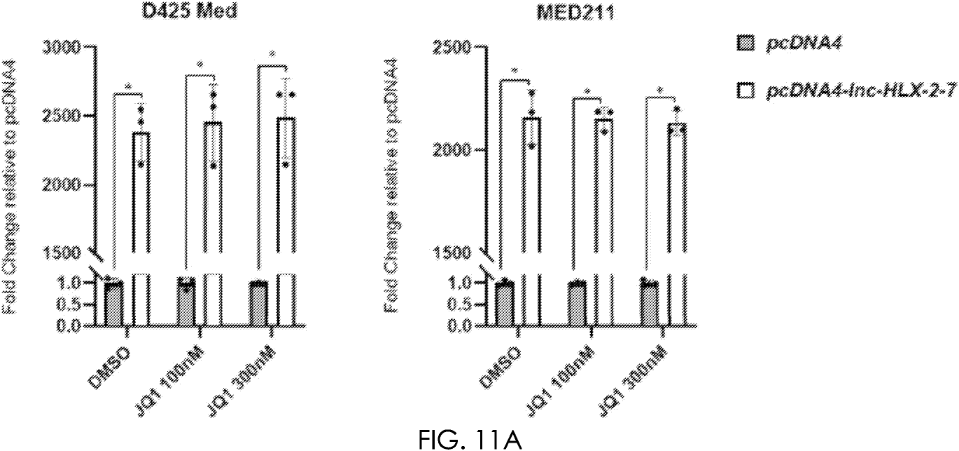

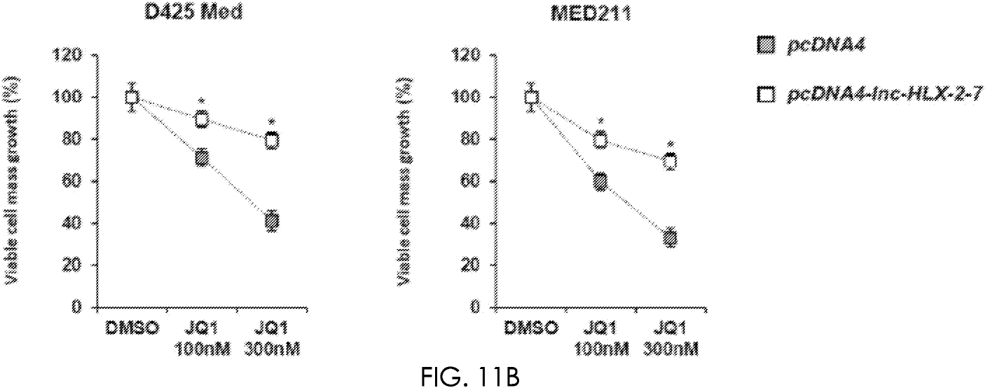

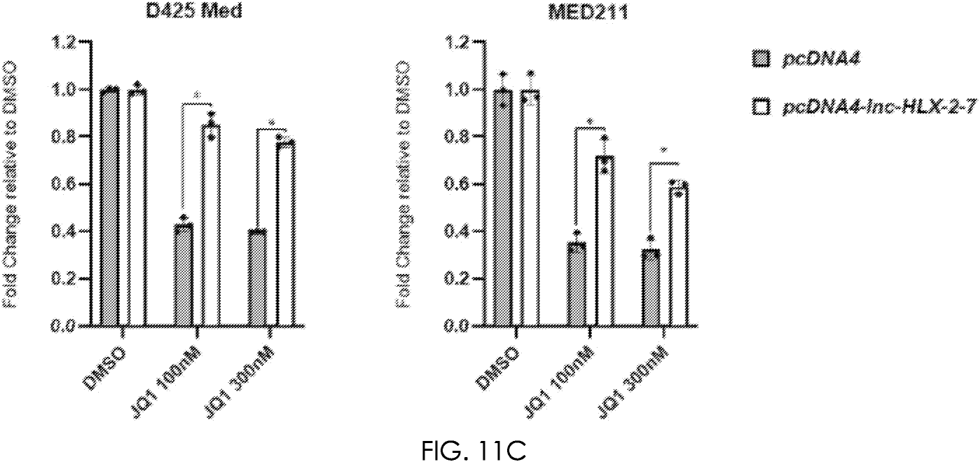

JQ1 Regulates lnc-HLX-2-7via MYC. Several previous studies have demonstrated that BRD4, a member of the bromodomain and extraterminal domain (BET) family, regulates MYC transcription and that JQ1 effectively suppresses cancer cell proliferation by inhibiting BRD4-mediated regulation of MYC in various types of cancer including MB.³⁰⁻³⁴ To test the JQ1 effect on lnc-HLX-2-7regulation, we treated D425 Med and MED211 cells with different doses (100 or 300 nM) of the drug. As shown in FIG. 4D, both MYC and lnc-HLX-2-7 were downregulated in D425 Med and MED211 cells. In addition, downregulation of lnc-HLX-2-7 by JQ1 was also confirmed in vivo (FIGS. 10). Interestingly, overexpression of lnc-HLX-2-7 suppressed cell growth inhibition and downregulation of MYC by JQ1 (FIGS. 11). Collectively, our results show that BRD4 inhibitors can be used to target MYC-mediated regulation of lnc- HLX-2-7 expression.

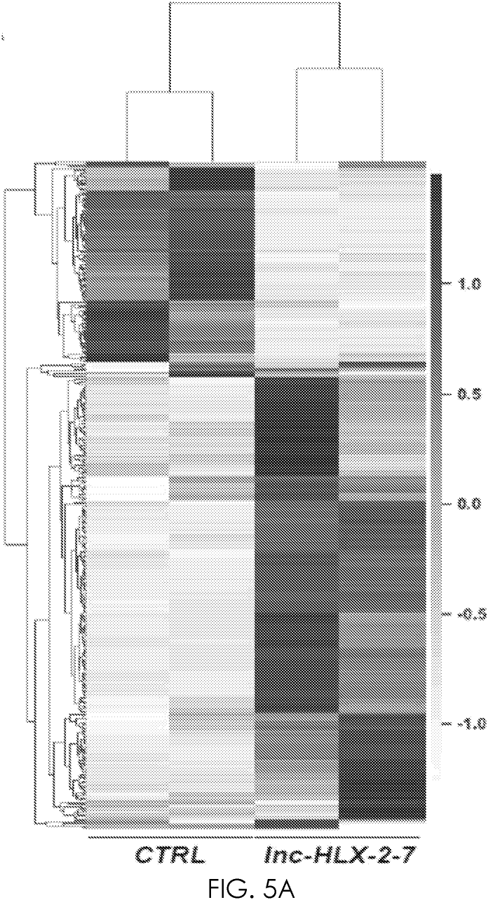

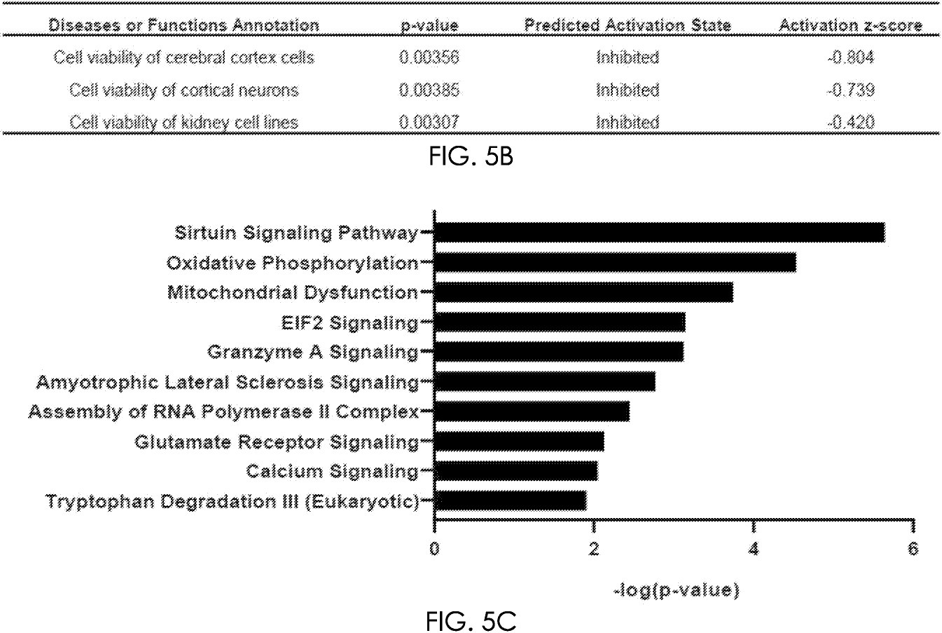

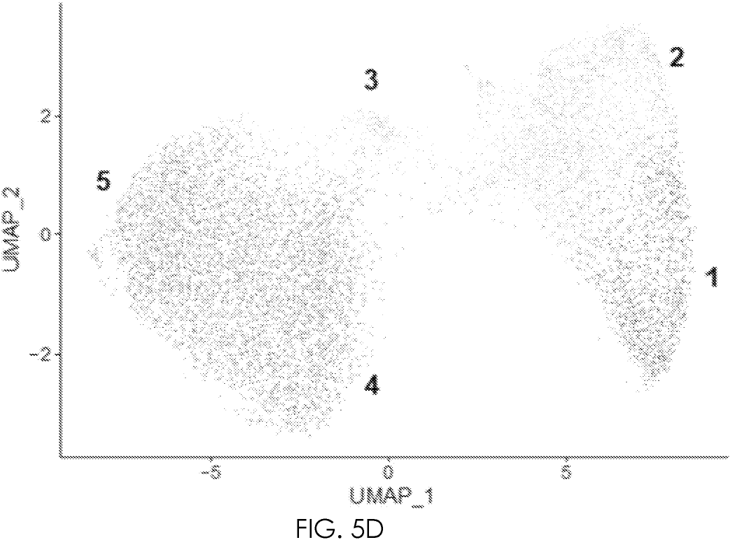

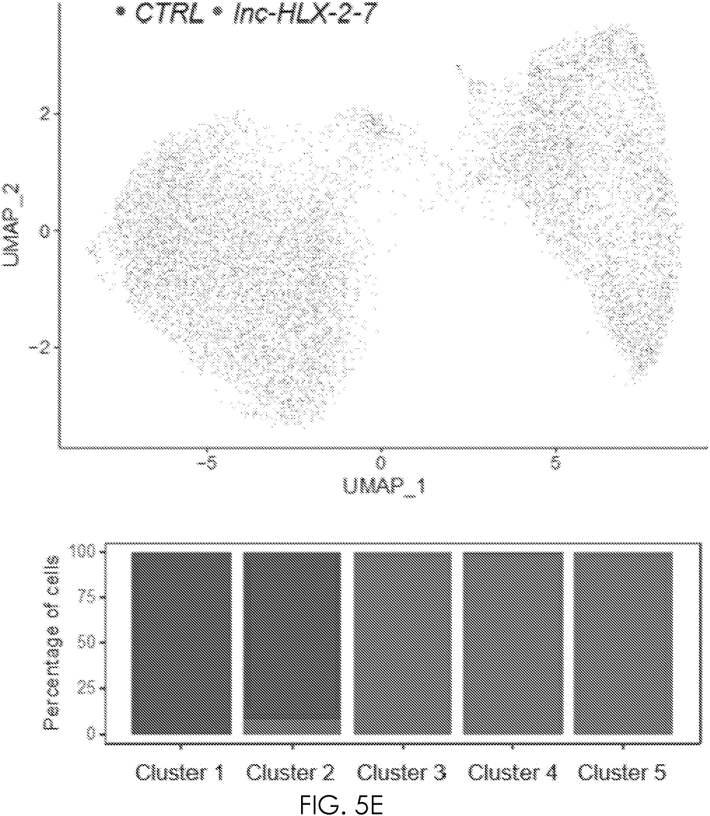

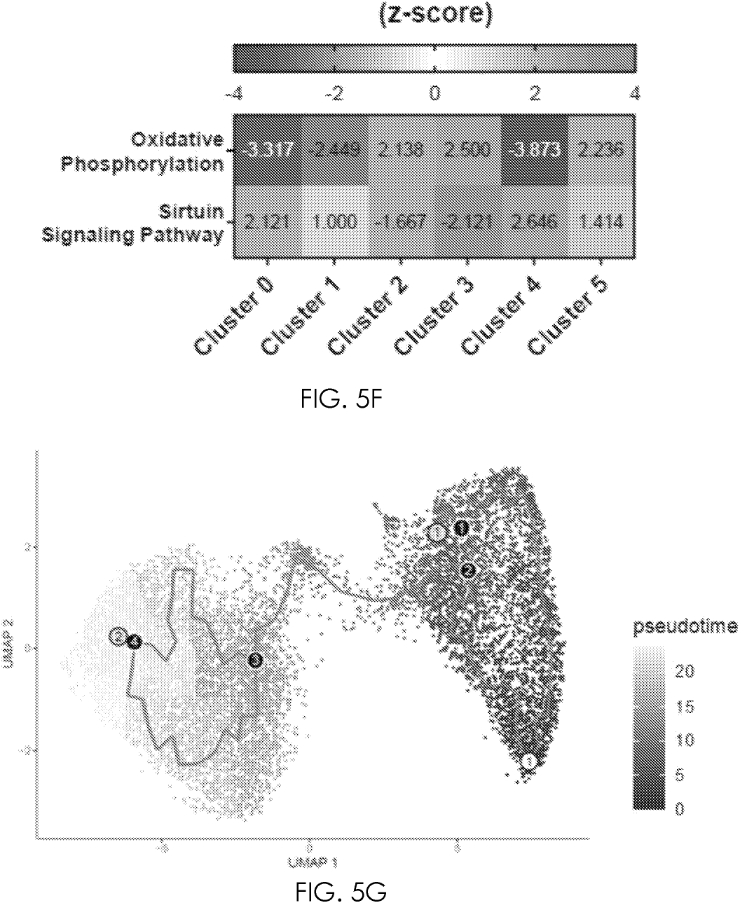

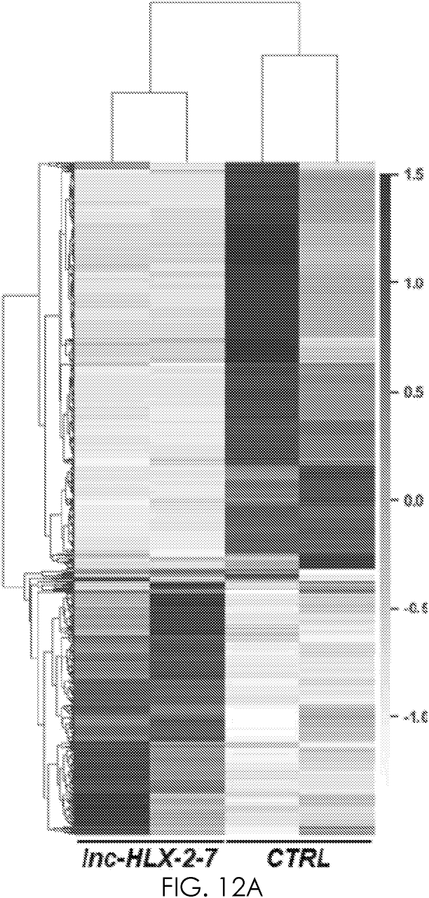

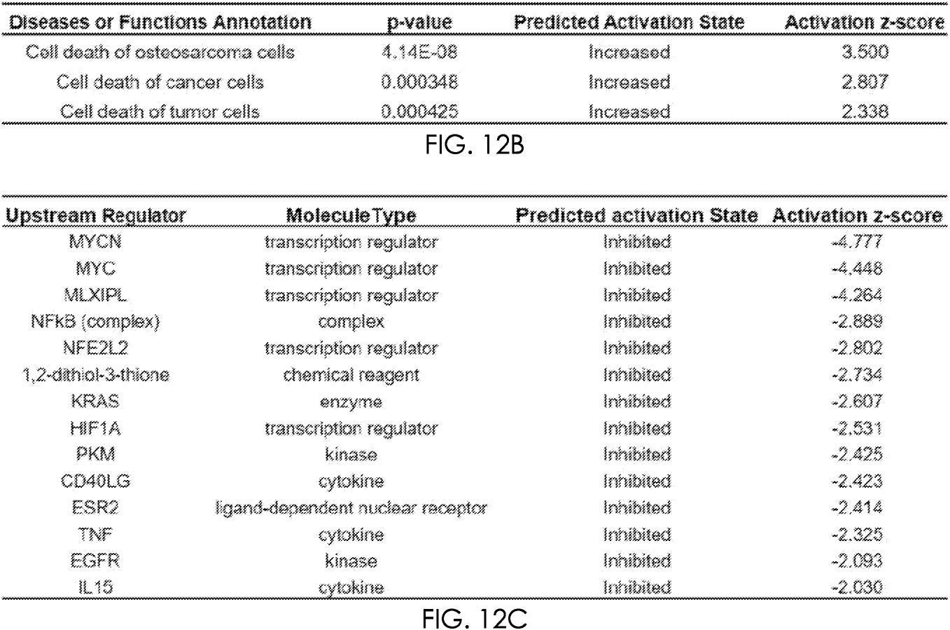

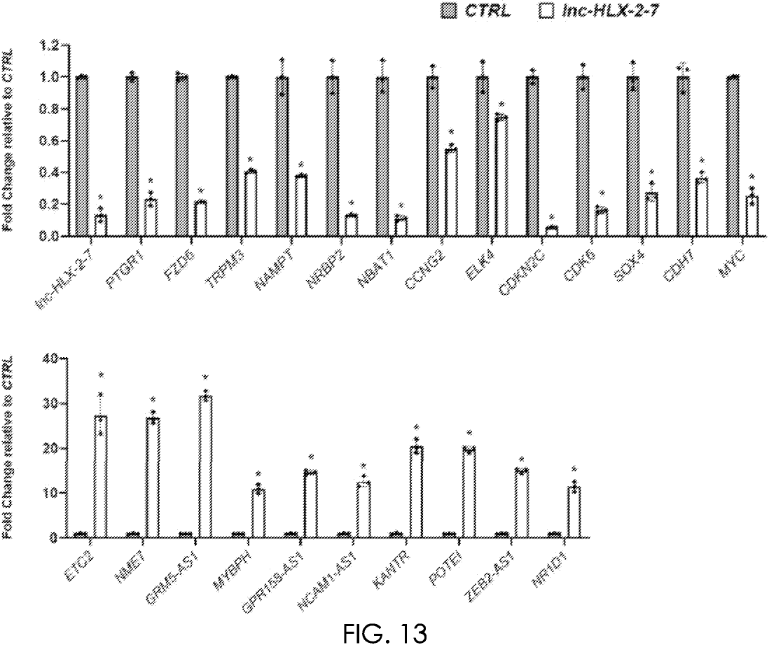

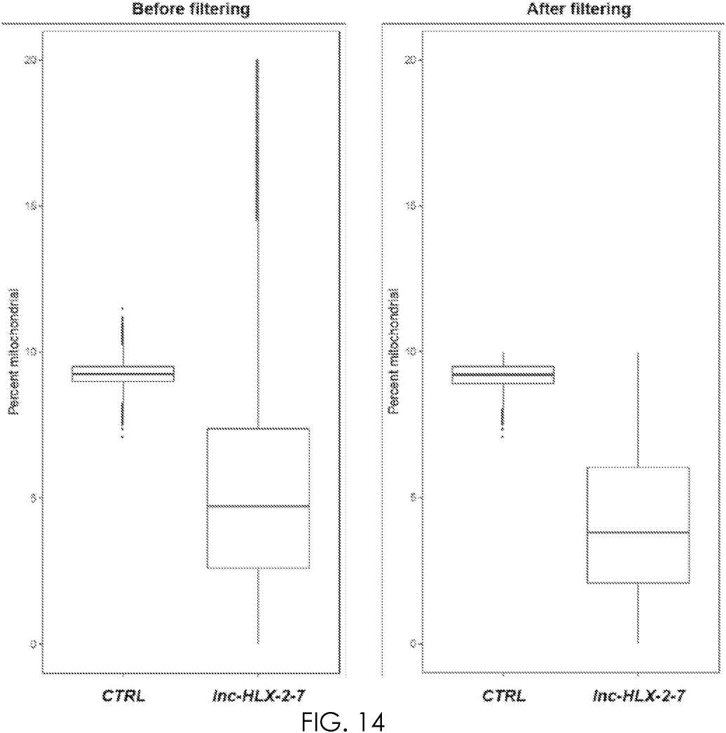

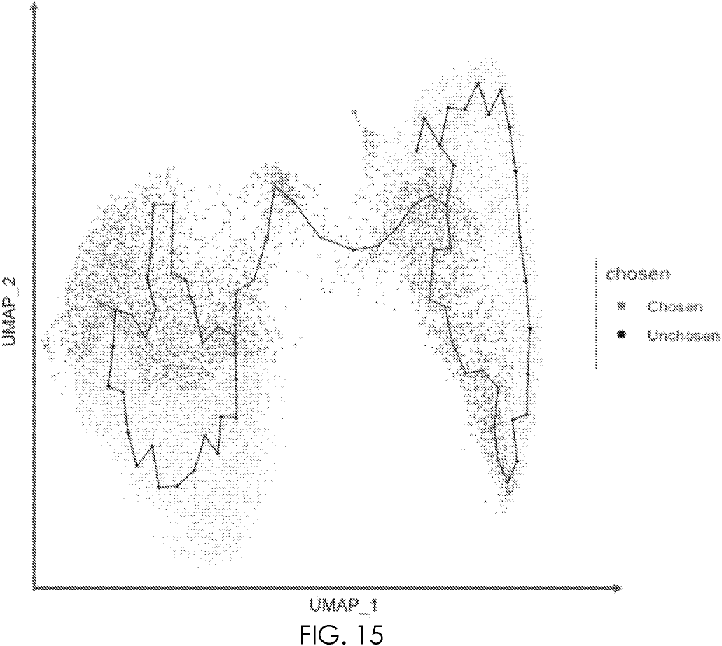

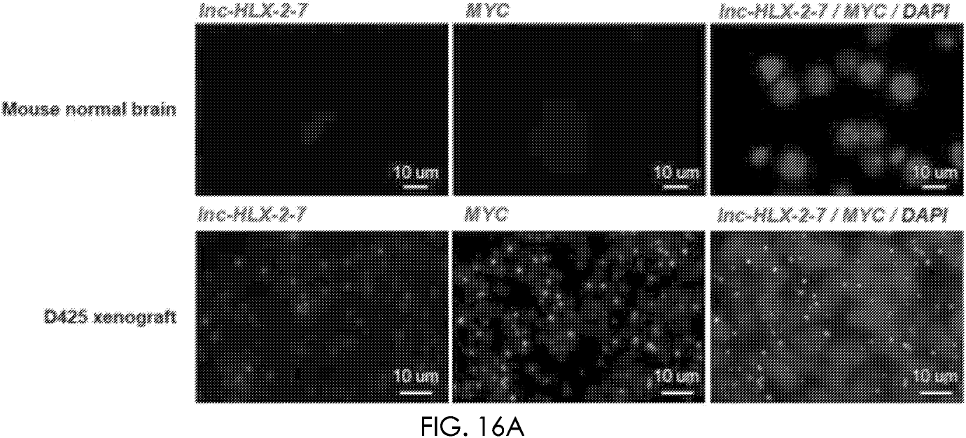

RNA Sequencing Detects lnc-HLX-2-7Interacting Genes and Pathways in Group 3 MBs. To gain further insights into the functional significance of lnc-HLX-2-7, gene expression was measured by RNAseq in D425 Med-lnc-HLX-2-7-sgRNA cells and in xenografts derived from them. Among 1033 genes with a significant change in expression (false discovery rate [FDR] < 0.05), 484 genes were upregulated and 549 genes were downregulated in cultured D425 Med-lnc-HLX-2- 7-sgRNA cells (FIG. 12A). IPA revealed that lnc-HLX-2-7knockdown preferentially affected genes associated with cell death (FIG. 12B). Of note, upstream regulator analysis showed that these genes contribute to important cancer pathways, including MYC, KRAS, HIF1A, and EGFR signaling (FIG. 12C). In xenografts, among 540 genes with a significant change in expression (FDR < 0.05), 409 genes were upregulated and 131 genes were downregulated (FIG. 5A). Differentially expressed genes detected by RNAseq and pathway analysis were validated by qRT-PCR (FIG. 13). IPA analysis revealed that lnc- HLX-2-7 knockdown preferentially regulated genes associated with cell viability (FIG. 5B). Canonical IPA pathway analysis showed that the pathways involved in important energy metabolism (oxidative phosphorylation, mitochondrial dysfunction, and sirtuin signaling pathways) were highly modulated by lnc-HLX-2-7 (FIG. 5C and Supplementary Table 4 (available online)). Xenograft tumors were further characterized by single cell RNA-seq. Subsequent to quality control, 3442 and 6193 single cells were obtained for D425 and Inc-HLX-2-7 deleted D425 respectively (FIG. 14). Integrated clustering of D425 control and Inc-HLX-2-7 depleted xenografts resulted in 5 clusters of single cells (FIG. 5D). Clusters 1 and 2 were almost entirely from D425 control xenografts, while clusters 3, 4, and 5 were almost exclusively from Inc-HLX-2-7 depleted xenografts (FIG. 5E). The top canonical pathways impacted in Inc-HLX-2-7-depleted single cell populations compared with D425 controls included the oxidative phosphorylation and sirtuin signaling pathways (FIG. 5F, Supplementary Tables 5, 6 (available online)), consistent with the bulk RNA-seq data. Based on our earlier result that D425 control and Inc-HLX-2-7 depleted single cells form separate clusters, we performed pseudotemporal ordering of cells using Monocle3³⁵ to identify genes responsible for the transition from the D425 control to Inc-HLX-2-7- depleted state (FIG. 5G). A graph path corresponding to transition of cells from cluster 1 through 5 was observed (FIG. 15). The top 370 genes contributing to the cell transition were selected based on Moran’s I and consisted of important genes involved in the development and malignancy of MB such as MYC, SOX4, CDK6, and CHD7 (Supplementary Table 7 (available online)).

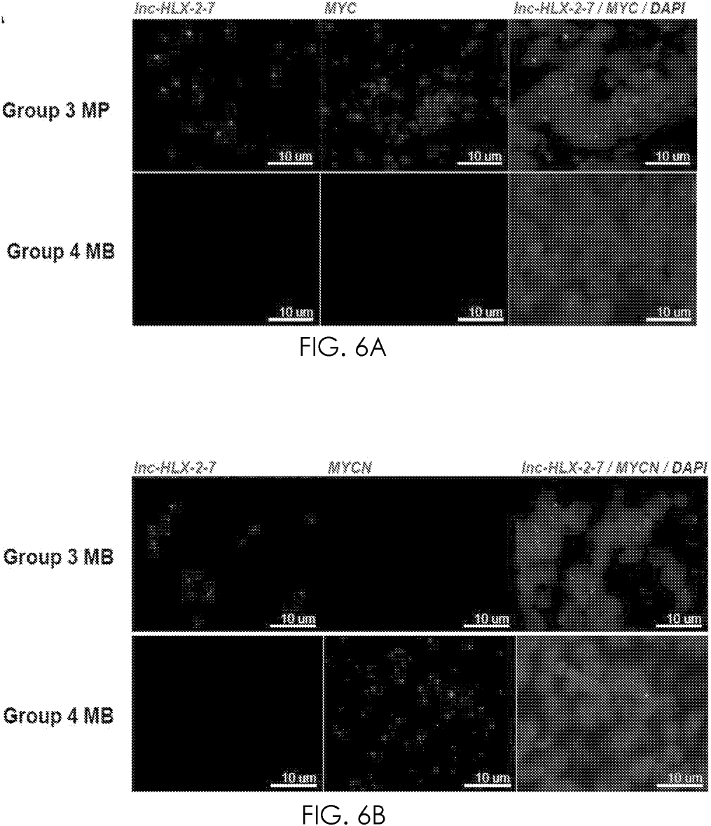

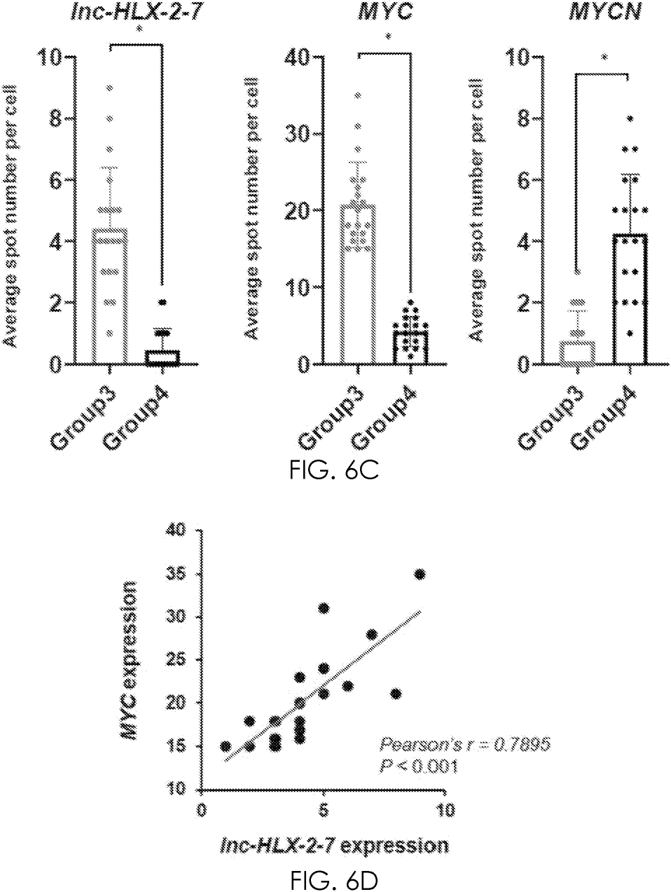

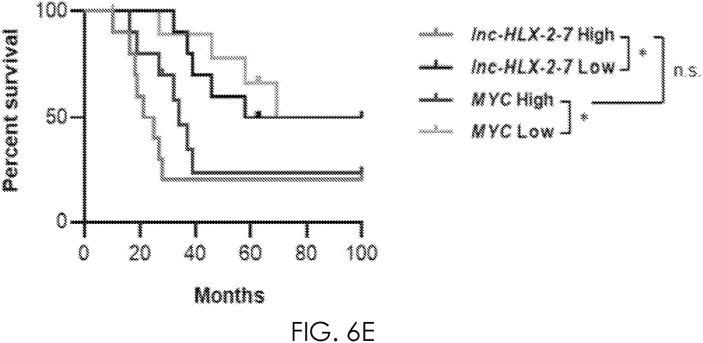

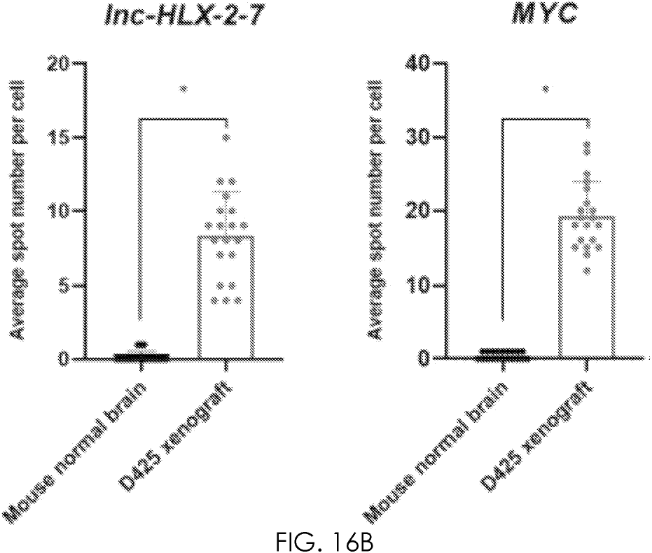

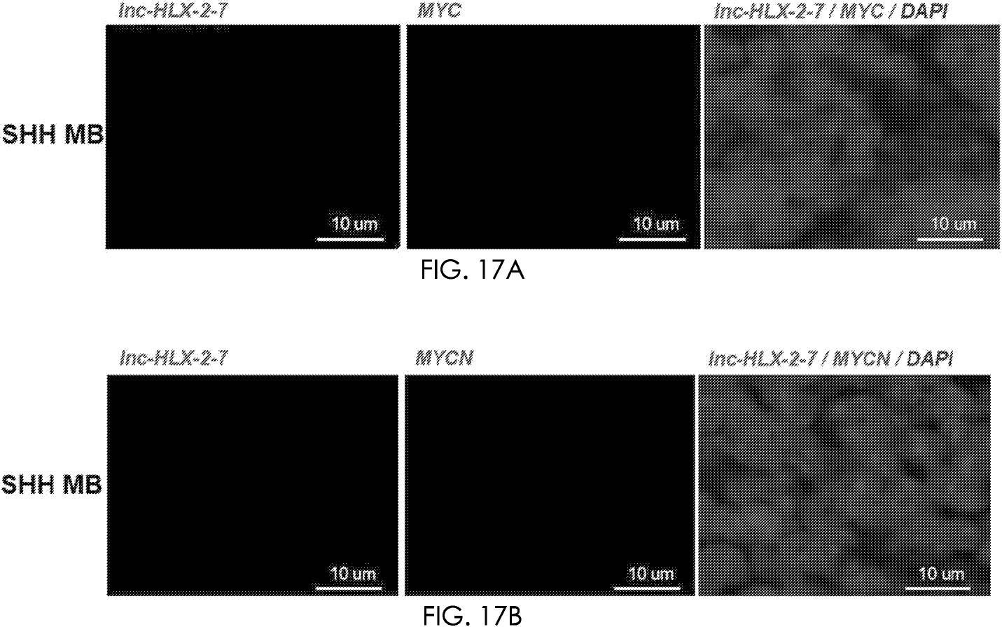

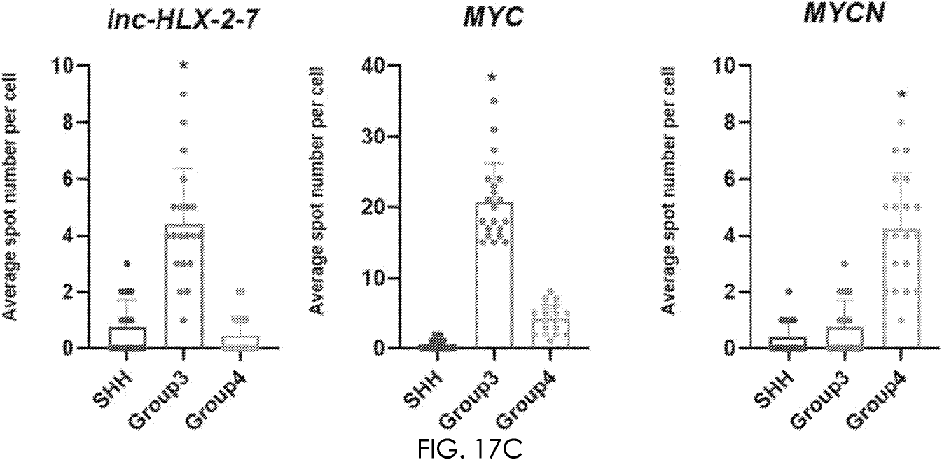

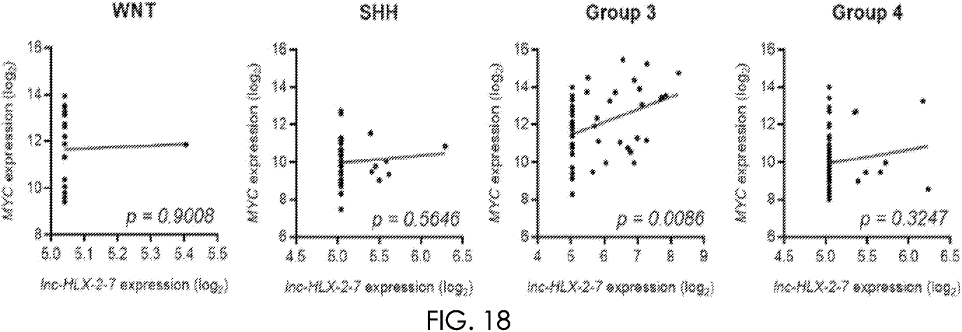

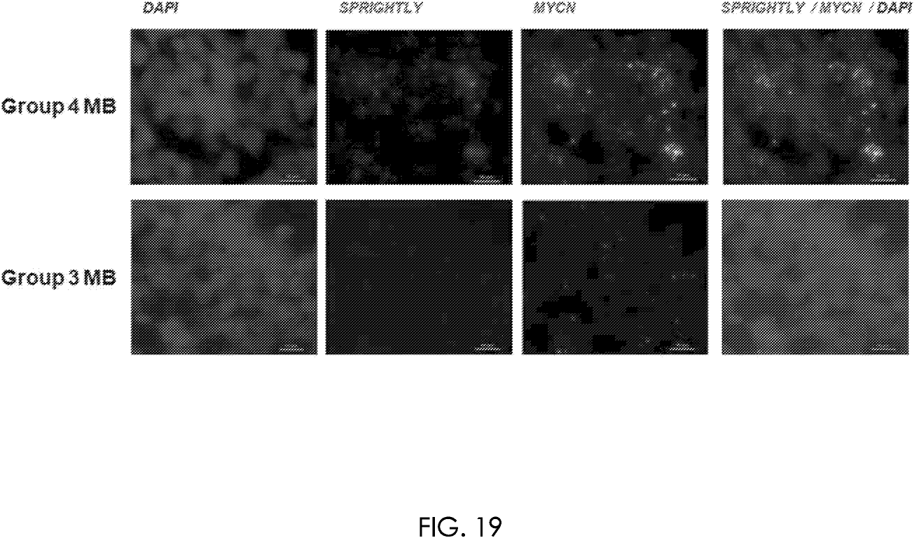

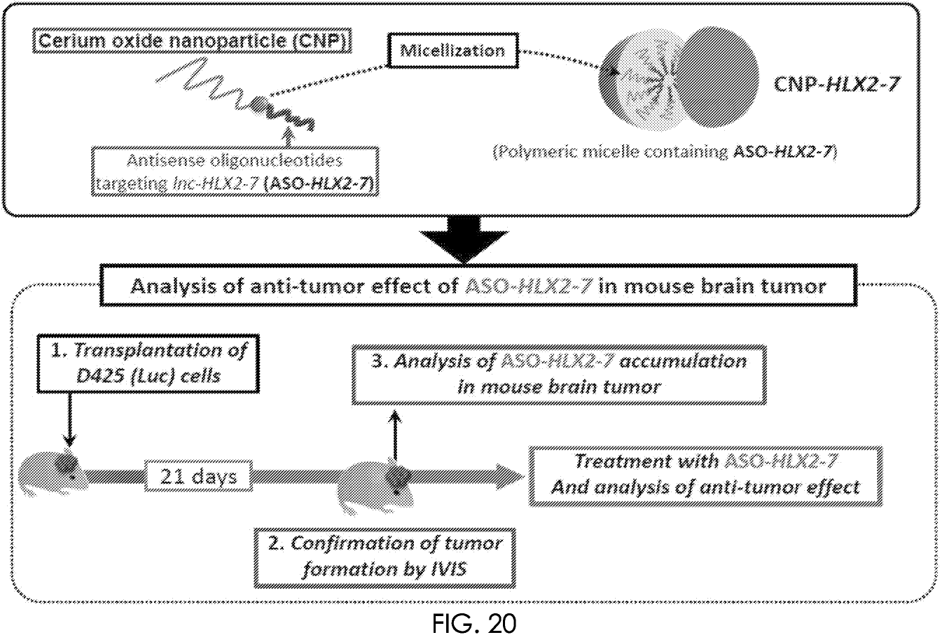

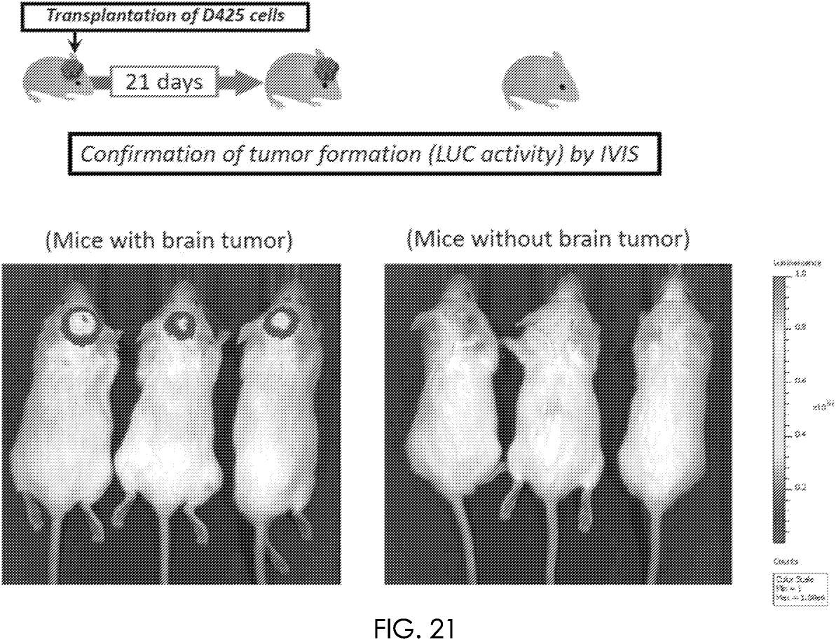

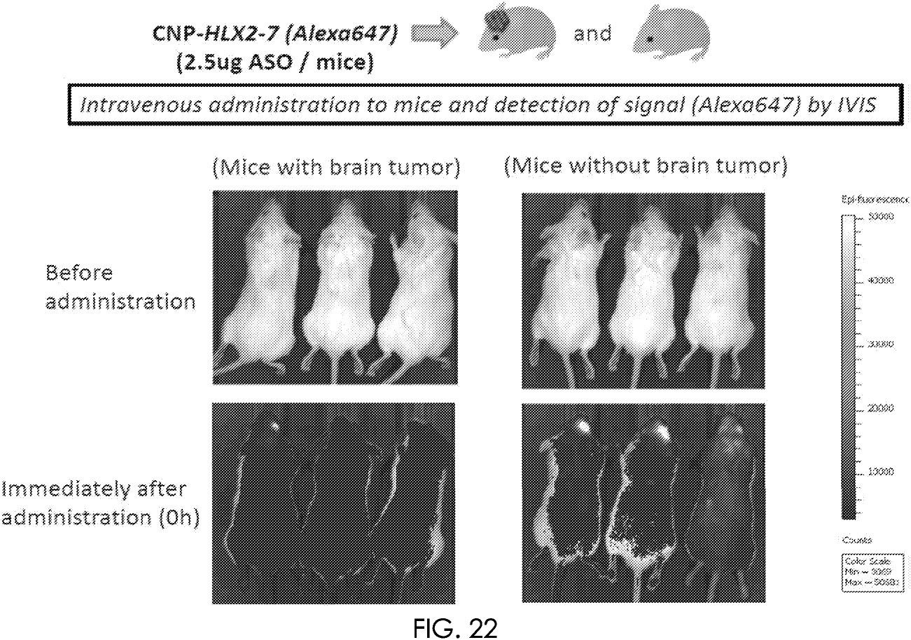

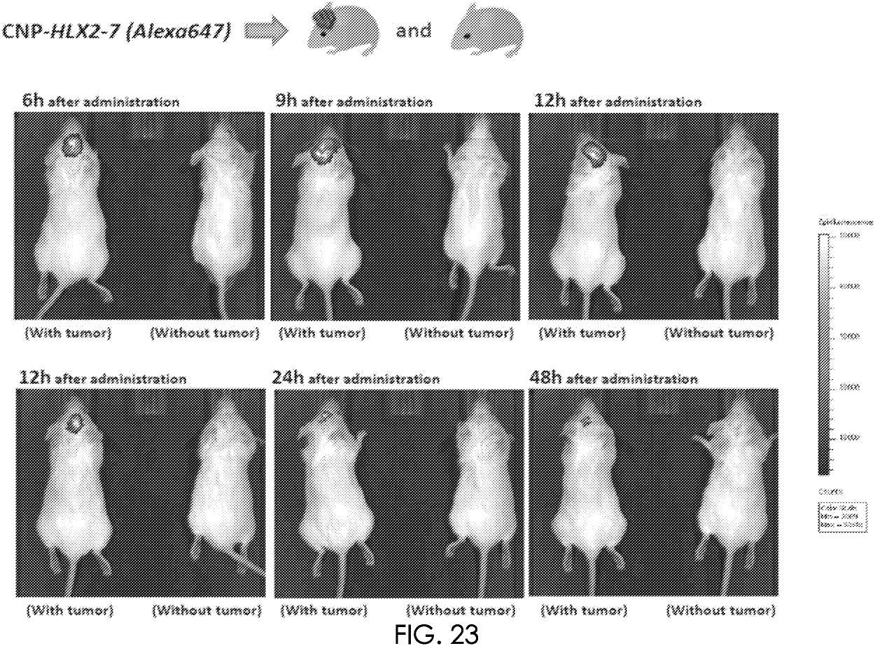

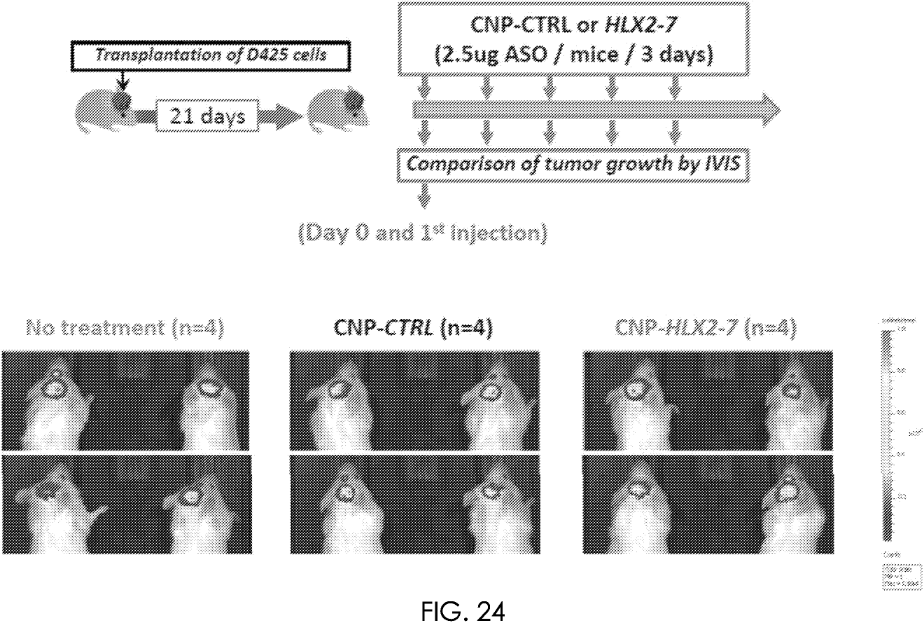

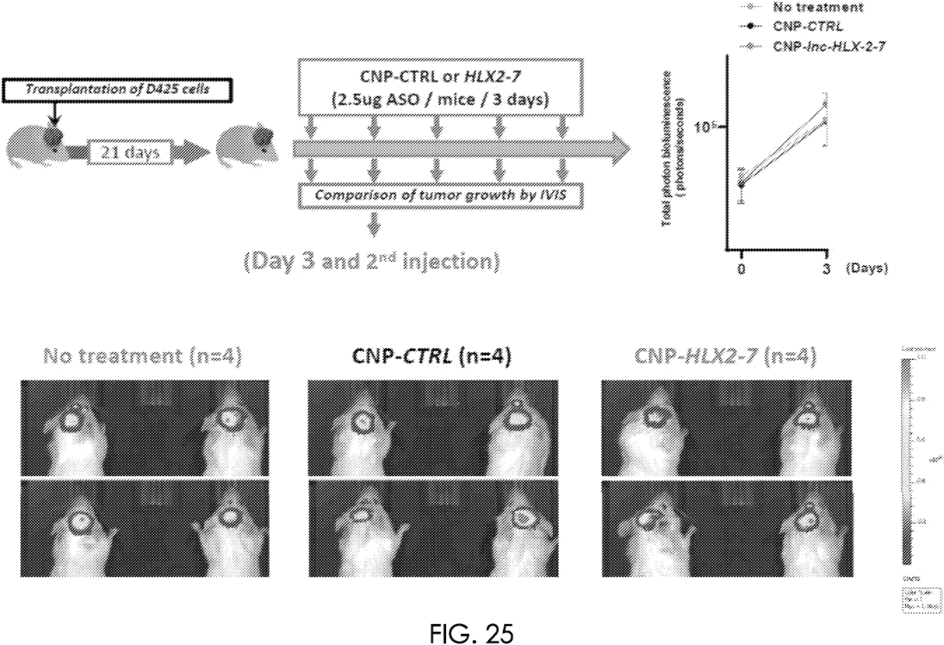

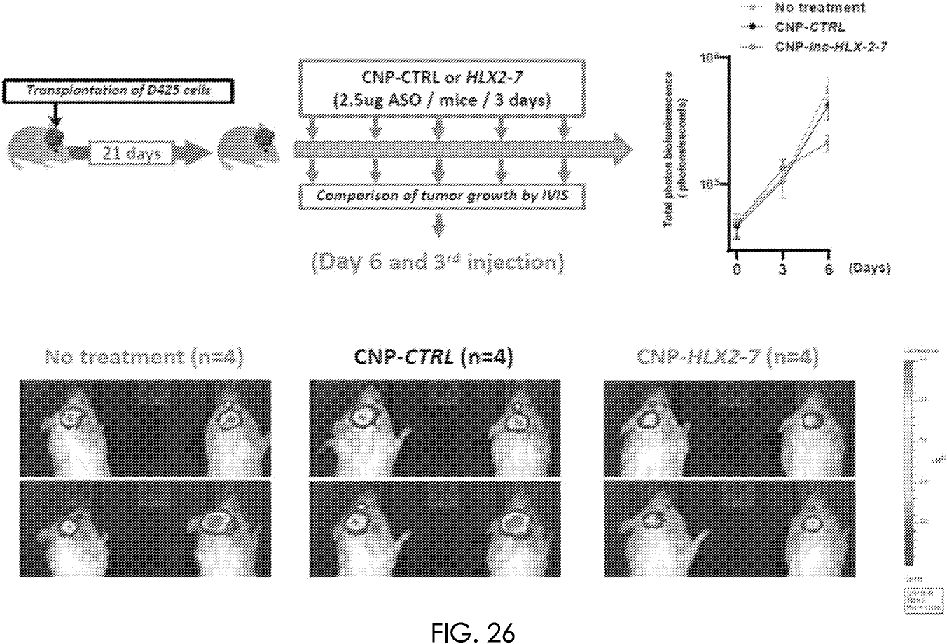

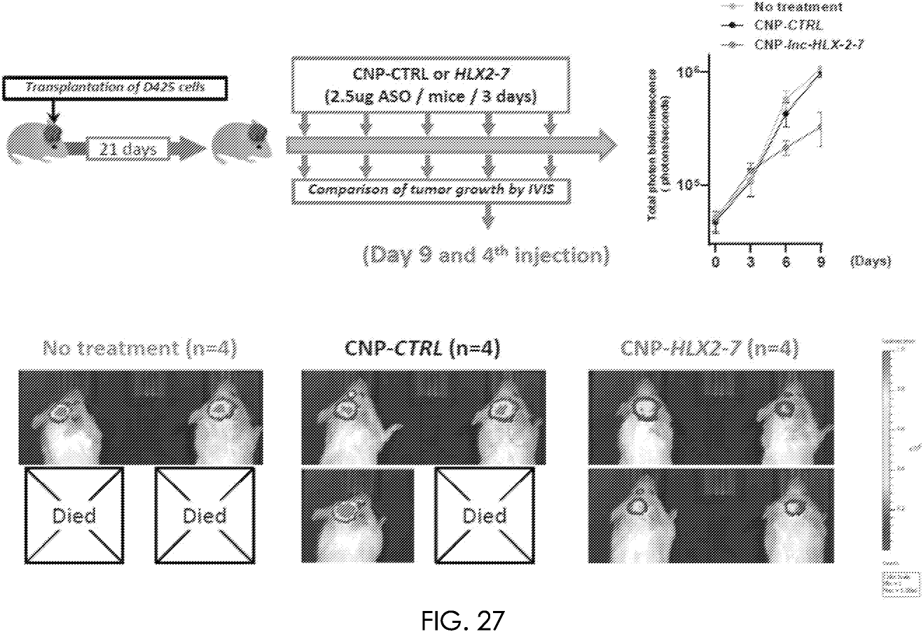

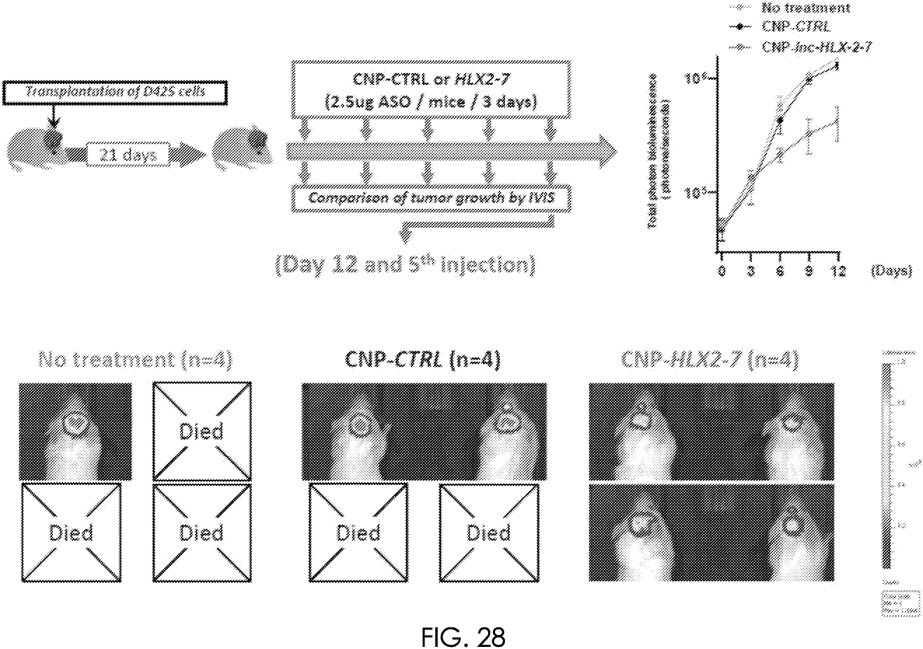

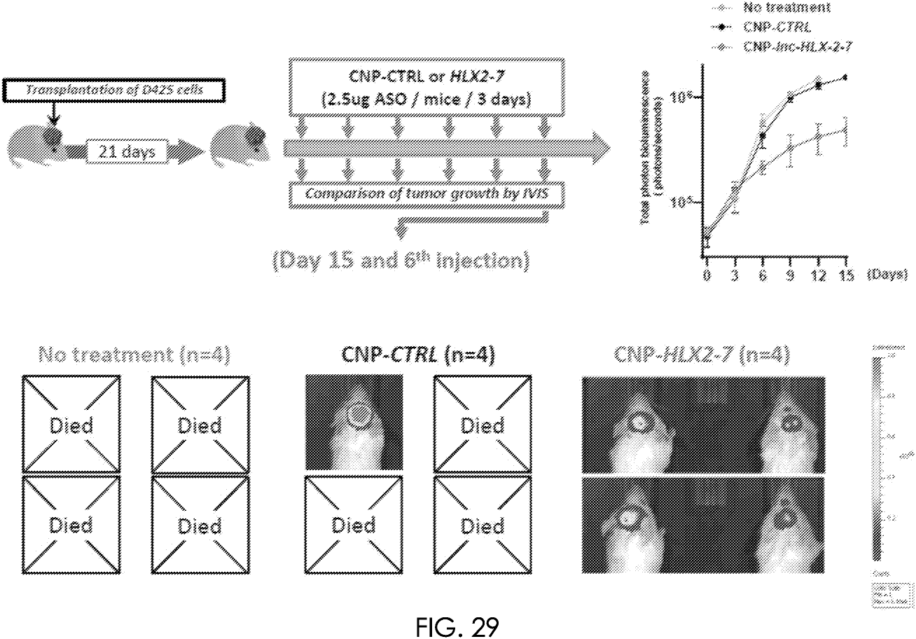

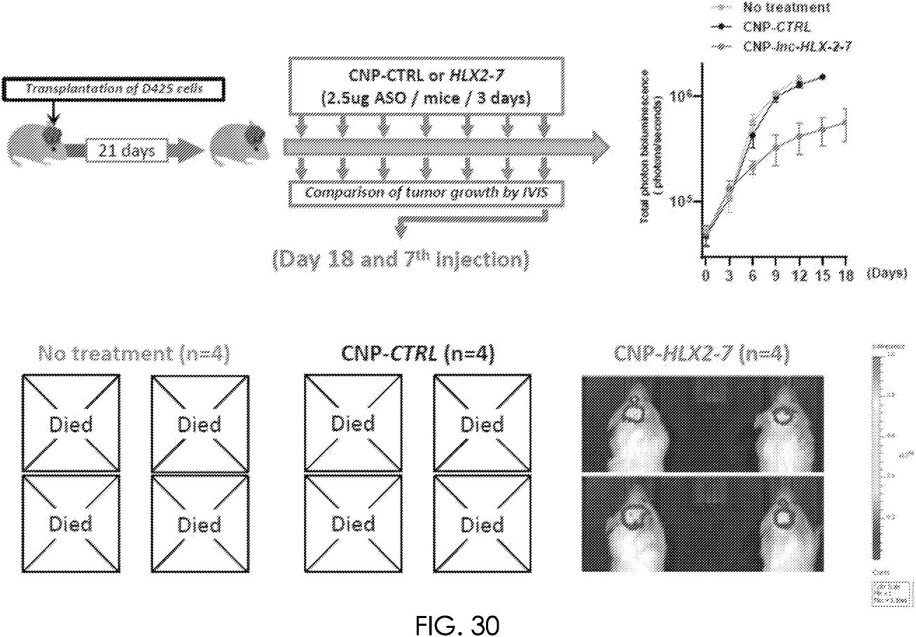

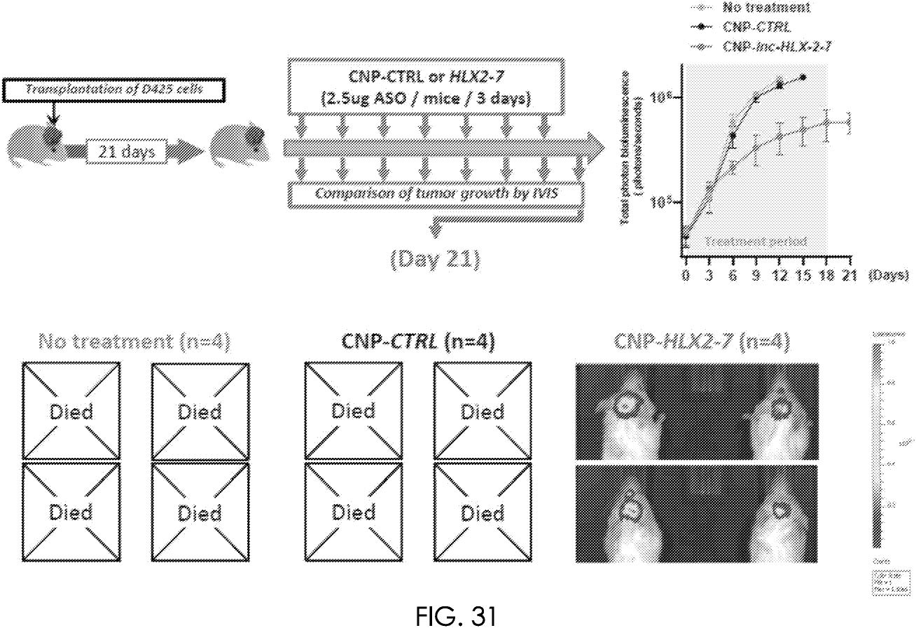

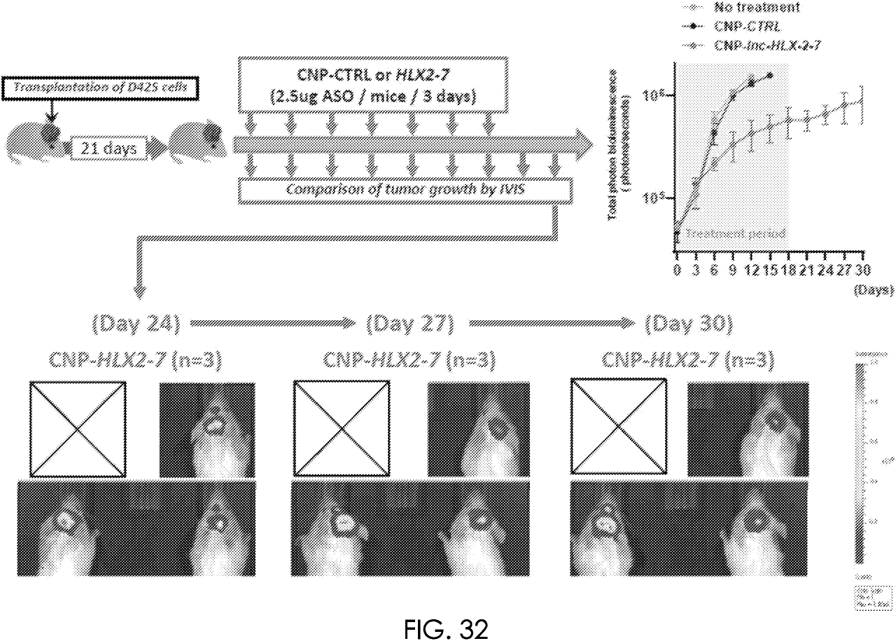

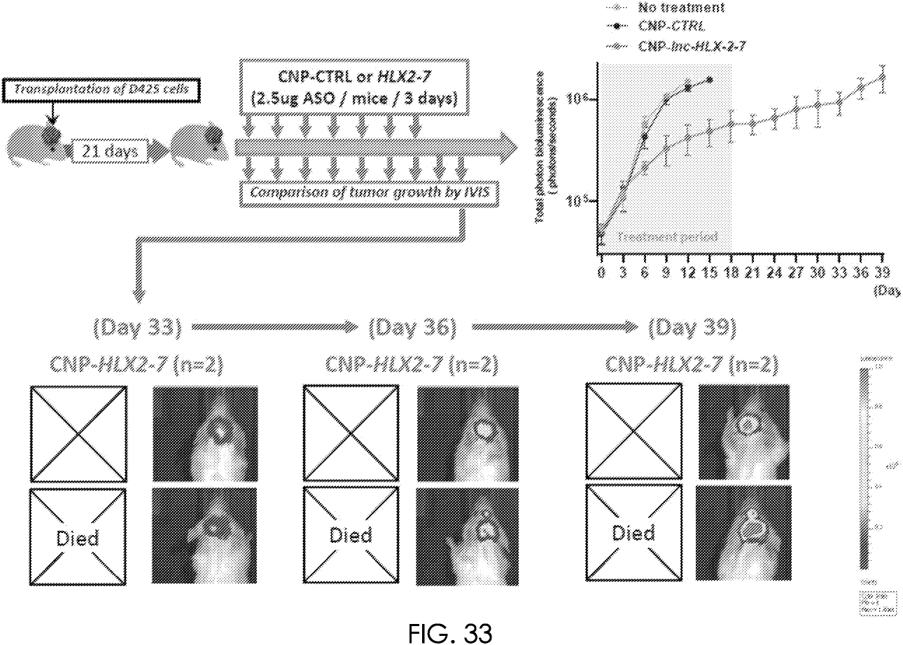

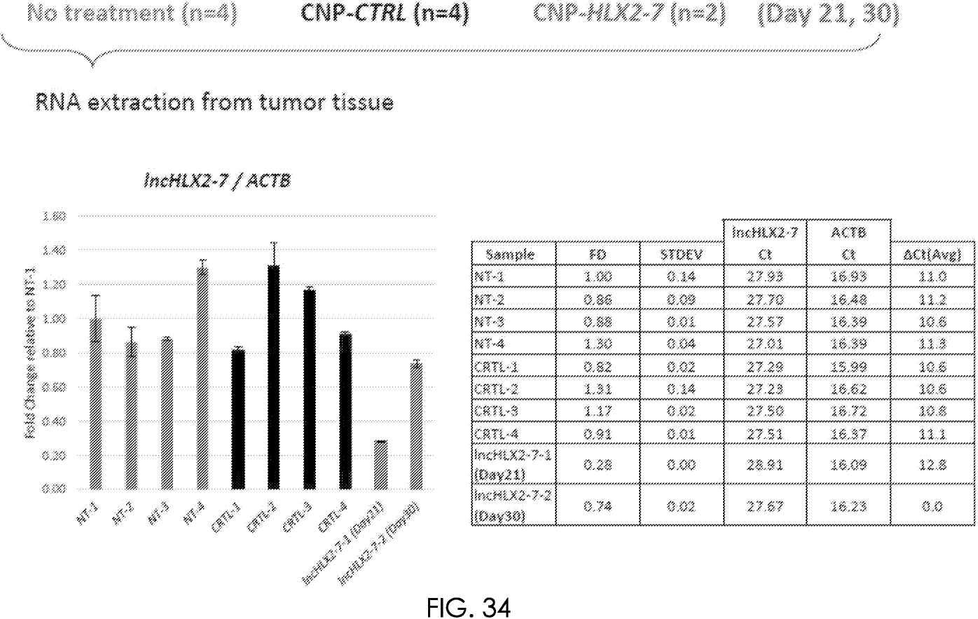

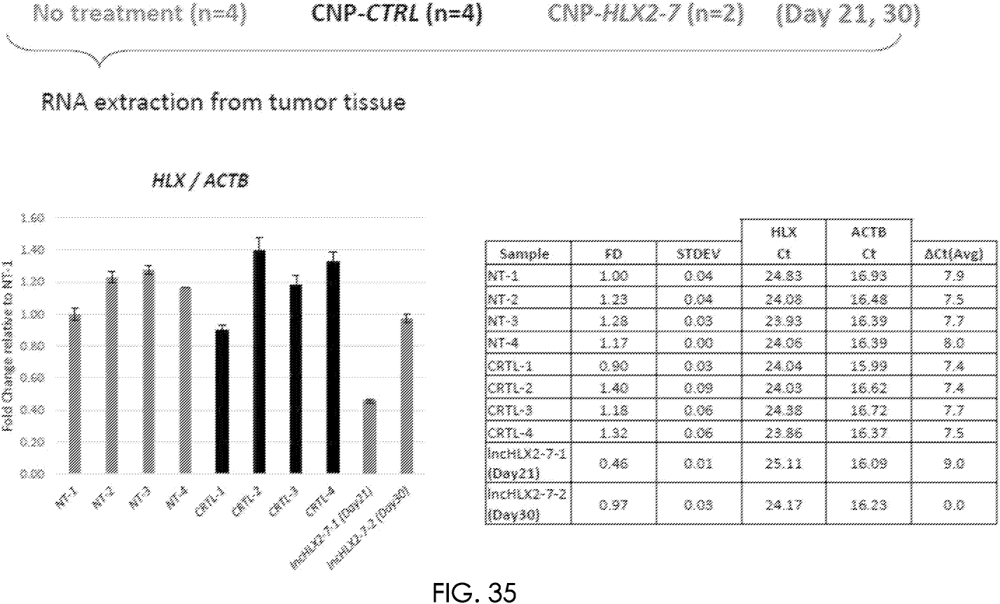

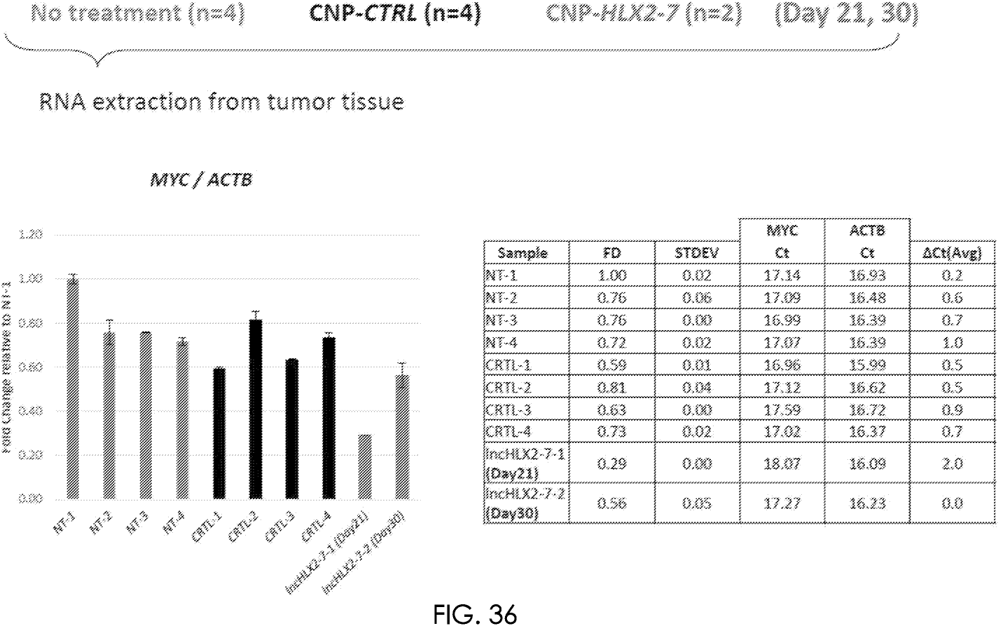

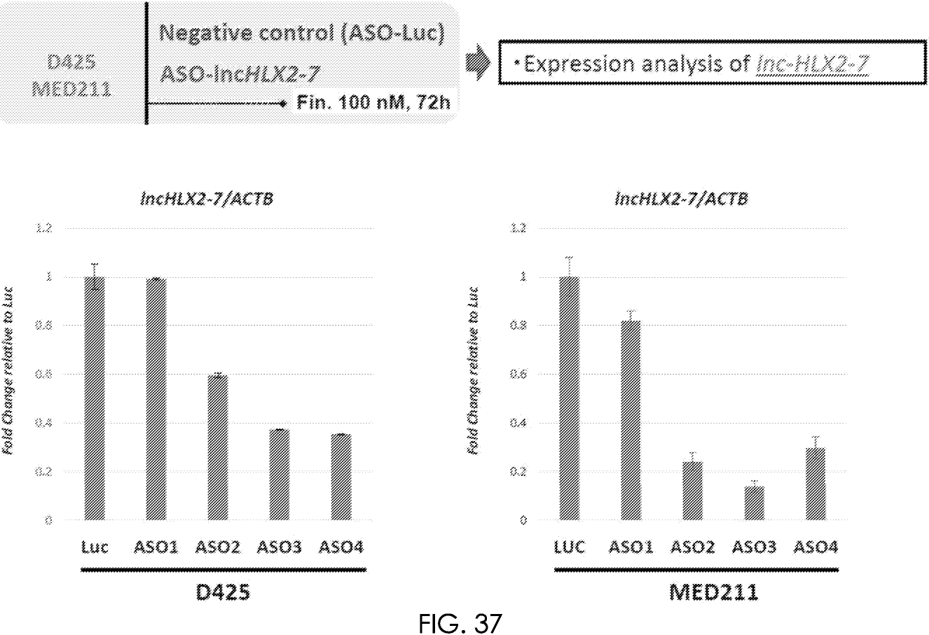

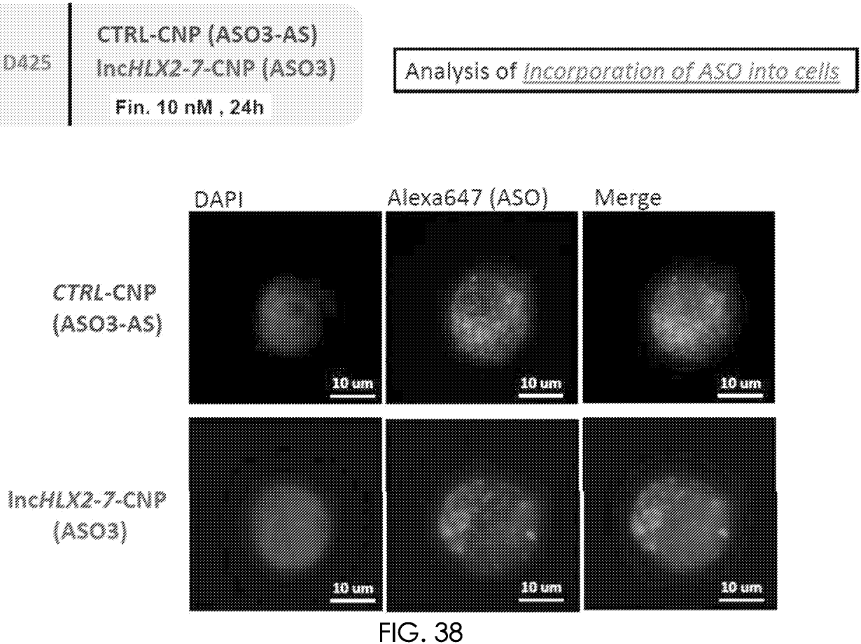

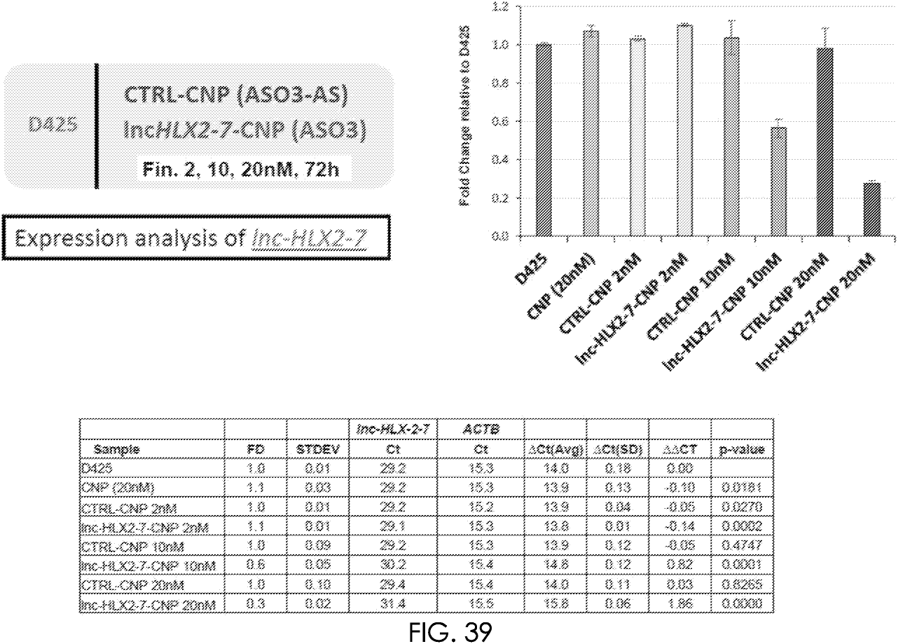

Lnc-HLX7 Expression Is Specific to Group 3 MBs. We next confirmed Group 3 specificity by visualizing Inc-HLX7 expression by RNA fluorescence in situ hybridization (FISH) in formalin-fixed paraffin-embedded tissue samples from D425 Med mouse xenografts and patients with MB. Lnc-HLX7 was expressed in D425 Med mouse xenografts but not normal brain (FIGS. 16), and Inc-HLX-2-7 was readily detected in all Group 3 MB samples but not in Group 4 MBs (FIGS. 6A, B). Quantitative analysis of the tissues further confirmed significantly higher Inc-HLX-2-7 expression in Group 3 MBs compared with Group 4 and SHH MBs with high sensitivity (95.0%) and specificity (95.0%, n = 20, P < 0.01; FIG. 6C and FIGS. 17). Importantly, Inc-HLX-2-7 expression was highly correlated withMYC expression in Group 3 MBs (n = 20, P < 0.01; FIG. 6D). This positive correlation between Inc-HLX-2-7 and MYC expression in Group 3 MB was further validated in RNA-seq data from 175 MB patients (FIG. 18). Finally, lnc- HLX-2-7 overexpression was associated with poor patient outcomes and mirrored that of MYC expression in Group 3 MB (FIG. 6E). Collectively, our analyses suggest that lnc- HLX-2-7 expression is specific to Group 3 MBs and can be detected using an assay readily applicable to the clinical setting.

### Discussion

The functions and clinical relevance of IncRNAs in MB are poorly described. Here we provide evidence that the IncRNA Inc-HLX-2-7 is clinically relevant and biologically functional in Group 3 MBs. Using publicly available patient-derived RNA-seq datasets, we discovered that lnc- HLX-2-7 expression is particularly high in Group 3 MBs compared with other groups. By depleting the expression of Inc-HLX-2-7 by CRISPR/Cas9 and ASOs, we showed both in vitro and in vivo that Inc-HLX-2-7 knockdown reduced proliferation and colony formation and increased apoptosis in MB.

The region encoded by Inc-HLX-2-7 has been reported as a Group 3 MB-specific enhancer region.²⁴ Therefore, ncRNAs transcribed from this region may function as enhancer RNAs, a class of IncRNAs synthesized at enhancers, and may regulate the expression of their surrounding genes. We found that Inc-HLX-2-7 positively regulated the expression of the adj acent HLX gene. Although the mechanism by which Inc-HLX-2-7 regulates HLX remains unclear, Inc-HLX-2-7 may function as an eRNA in this context. HLX has recently been shown to be a key gene mediating BET inhibitor responses and resistance in Group 3 MBs.³⁶ In this study, we discovered that Inc-HLX-2-7 controls HLX expression and contributes to MB cell proliferation, so it is possible that it may influence BET inhibitor resistance. In addition, our results show that the MYC oncogene regulates Inc-HLX-2-7 expression. A recent report suggests that the small molecule JQ1, a BET inhibitor that disrupts interactions with MYC, could be a therapeutic option to treat Group 3 MBs.³⁷ However, Group 3 MB tumors may also become resistant to BET inhibitor through mutations in the BRD4 gene, and transcription factors like MYC and HLX are poor therapeutic targets with short half-lives and pleiotropic properties.³⁸ We postulate that Inc-HLX-2-7 inhibition may provide a novel solution to BET inhibitor resistance or amplify the effects of BET inhibitors, a hypothesis that requires further investigation.

Recent evidence shows that HLX directly regulates several metabolic genes and controls mitochondrial biogenesis.³⁹ In the present study, we demonstrate that Inc-HLX-2-7 modulated oxidative phosphorylation, mitochondrial dysfunction, and sirtuin signaling pathways in intracranial xenograft models. These findings suggest that Inc-HLX-2-7 contributes to the metabolic state of Group 3 MBs by regulating HLX expression. This newly discovered link between Inc-HLX-2-7 and metabolism may have important therapeutic implications.

Group 3 and Group 4 MBs display clinical and genetic overlap, with similar anatomic location and presence of isochromosome 17q, so it is not currently possible to distinguish them without applying multigene expression or methylation profiling. Lnc-HLX-2-7 may represent a useful single molecular marker that could distinguish Group 3 from Group 4 MBs. Furthermore, RNA-FISH using probes targeting Inc-HLX-2-7, a technique readily applicable in clinical laboratories, readily discriminated Group 3 from Group 4 MBs. It was recently shown through a combined analysis of Group 3 and 4 MBs that they can be subdivided into 8 molecular subtypes, designated I to VIII.²⁰ Subtypes II and III are characterized by amplification of the MYC oncogene and are associated with the poorest prognosis.⁴° We found that Inc-HLX-2-7 is specifically expressed in subtype II and III MBs. These findings strongly suggest that Inc-HLX-2-7 may be an ideal prognostic marker in Group 3 MBs.

In conclusion, we show that the IncRNA Inc-HLX-2-7 is clinically and functionally relevant in Group 3 MBs. Future studies will determine the mechanism by which Inc-HLX-2-7 promotes MB tumorigenesis. Together, our findings support the hypothesis that IncRNAs, and lnc- HLX-2-7 in particular, are functional in human MBs and may offer promising future opportunities for diagnosis and therapy.

### References

1. Northcott PA, Jones DT, Kool M, et al. Medulloblastomics: the end of the beginning. Nat Rev Cancer. 2012;12(12):818-834.

2. Northcott PA, Shih DJ, Peacock J, et al. Subgroup-specific structural variation across 1,000 medulloblastoma genomes. Nature. 2012;488(7409):49-56.

3. Jones DT, Jäger N, Kool M, et al. Dissecting the genomic complexity underlying medulloblastoma. Nature. 2012;488(7409):100-105.

4. Taylor MD, Northcott PA, Korshunov A, et al. Molecular subgroups of medulloblastoma: the current consensus. Acta Neuropathol. 2012;123(4):465-472.

5. Wahlestedt C. Targeting long noncoding RNA to therapeutically upregulate gene expression. Nat Rev Drug Discov. 2013;12(6):433-446.

6. Schmitt AM, Chang HY. Long noncoding RNAs in cancer pathways. Cancer Cell. 2016;29(4):452-463.

7. Quinn JJ, Chang HY. Unique features of long noncoding RNA biogenesis and function. Nat Rev Genet. 2016;17(1):47-62.

8. Khaitan D, Dinger ME, Mazar J, et al. The melanoma-upregulated long noncoding RNA SPRY4-IT1 modulates apoptosis and invasion. Cancer Res. 2011;71(11):3852-3862.

9. Sahakyan A, Yang Y, Plath K. The role of Xist in X-chromosome dosage compensation. Trends Cell Biol. 2018;28(12):999-1013.

10. Trimarchi T, Bilal E, Ntziachristos P, et al. Genome-wide mapping and characterization of Notch-regulated long noncoding RNAs in acute leukemia. Cell. 2014;158(3):593-606.

11. Katsushima K, Natsume A, Ohka F, et al. Targeting the Notch-regulated noncoding RNA TUG1 for glioma treatment. Nat Commun. 2016;7:13616.

12. Long Y, Wang X, Youmans DT, Cech TR. How do IncRNAs regulate transcription? Sci Adv. 2017;3(9):eaao2110.

13. Esteller M. Noncoding RNAs in human disease. Nat Rev Genet. 2011;12(12):861-874.

14. Wapinski O, Chang HY. Long noncoding RNAs and human disease. Trends Cell Biol. 2011;21(6):354-361.

15. Varon M, Levy T, Mazor G, et al. The long noncoding RNA TP73-AS1 promotes tumorigenicity of medulloblastoma cells. Int J Cancer. 2019;145(12):3402-3413.

16. Joshi P, Katsushima K, Zhou R, et al. The therapeutic and diagnostic potential of regulatory noncoding RNAs in medulloblastoma. Neurooncol Adv. 2019;1(1):vdz023.

17. Zhang Y, Wang T, Wang S, et al. Nkx2-2as suppression contributes to the pathogenesis of sonic hedgehog medulloblastoma. Cancer Res. 2018;78(4):962-973.

18. Gao R, Zhang R, Zhang C, Zhao L, Zhang Y. Long noncoding RNA CCAT1 promotes cell proliferation and metastasis in human medulloblastoma via MAPK pathway. Tumori. 2018;104(1):43-50.

19. Iyer MK, Niknafs YS, Malik R, et al. The landscape of long noncoding RNAs in the human transcriptome. Nat Genet. 2015;47(3): 199-208.

20. Northcott PA, Buchhalter I, Morrissy AS, et al. The wholegenome landscape of medulloblastoma subtypes. Nature. 2017;547(7663):311-317.

21. Lee B, Sahoo A, Marchica J, et al. The long noncoding RNA SPRIGHTLY acts as an intranuclear organizing hub for pre-mRNA molecules. Sci Adv. 2017:3(5):e1602505.

22. Uszczynska-Ratajczak B, Lagarde J, Frankish A, Guigó R, Johnson R. Towards a complete map of the human long non-coding RNA transcriptome. Nat Rev Genet. 2018;19(9):535-548.

23. Joshi P, Jallo G, Perera RJ. In silico analysis of long non-coding RNAs in medulloblastoma and its subgroups. Neurobiol Dis. 2020;141:104873.

24. Lin CY, Erkek S, Tong Y, et al. Active medulloblastoma enhancers reveal subgroup-specific cellular origins. Nature. 2016;530(7588):57-62.

25. Engreitz JM, Haines JE, Perez EM, et al. Local regulation of gene expression by IncRNA promoters, transcription and splicing. Nature. 2016;539(7629):452-455.

26. Joung J, Engreitz JM, Konermann S, et al. Genome-scale activation screen identifies a IncRNA locus regulating a gene neighbourhood. Nature. 2017;548(7667):343-346.

27. Toiber D, Leprivier G, Rotblat B. Long noncoding RNA: noncoding and not coded. Cell Death Discov. 2017;3:16104.

28. Cavalli FMG, Remke M, Rampasek L, et al. Intertumoral heterogeneity within medulloblastoma subgroups. Cancer Cell. 2017;31(6):737-754.e736.

29. Mathelier A, Wasserman WW. The next generation of transcription factor binding site prediction. PLoS Comput Biol. 2013;9(9):e1003214.

30. Bolin S, Borgenvik A, Persson CU, et al. Combined BET bromodomain and CDK2 inhibition in MYC-driven medulloblastoma. Oncogene. 2018;37(21):2850-2862.

31. Venkataraman S, Alimova I, Balakrishnan I, et al. Inhibition of BRD4 attenuates tumor cell self-renewal and suppresses stem cell signaling in MYC driven medulloblastoma. Oncotarget. 2014;5(9):2355-2371.

32. Henssen A, Thor T, Odersky A, et al. BET bromodomain protein inhibition is a therapeutic option for medulloblastoma. Oncotarget. 2013;4(11):2080-2095.

33. Shi X, Liu C, Liu B, Chen J, Wu X, Gong W. JQ1: a novel potential therapeutic target. Pharmazie. 2018;73(9):491-493.

34. Delmore JE, Issa GC, Lemieux ME, et al. BET bromodomain inhibition as a therapeutic strategy to target c-Myc. Cell. 2011;146(6):904-917.

35. Trapnell C, Cacchiarelli D, Grimsby J, et al. The dynamics and regulators of cell fate decisions are revealed by pseudotemporal ordering of single cells. Nat Biotechnol. 2014;32(4):381-386.

36. Bandopadhayay P, Piccioni F, O’Rourke R, et al. Neuronal differentiation and cell-cycle programs mediate response to BET-bromodomain inhibition in MYC-driven medulloblastoma. Nat Commun. 2019; 10(1):2400.

37. Bandopadhayay P, Bergthold G, Nguyen B, et al. BET bromodomain inhibition of MYC-amplified medulloblastoma. Clin Cancer Res. 2014;20(4):912-925.

38. McKeown MR, Bradner JE. Therapeutic strategies to inhibit MYC. Cold Spring Harb Perspect Med. 2014;4(10).

39. Piragyte I, Clapes T, Polyzou A, et al. A metabolic interplay coordinated by HLX regulates myeloid differentiation and AML through partly overlapping pathways. Nat Commun. 2018;9(1):3090.

40. Sharma T, Schwalbe EC, Williamson D, et al. Second-generation molecular subgrouping of medulloblastoma: an international metaanalysis of Group 3 and Group 4 subtypes. Acta Neuropathol. 2019;138(2):309-326.

### Supplemental Materials and Methods

RNA samples. RNA samples were isolated from normal human cerebellum (BioChain, Newark, CA), MB cell lines, and patient-derived xenografts (PDXs). The cell lines DAOY, ONS76, D283 Med, D341 Med, D458 Med, MB002, and HD-MB03 were maintained in the Wechsler-Reya and Raabe labs. The PDXs DMB006, DMB012, RCMB28, RCMB32, RCMB38, RCMB40, RCMB45, and RCMB51 were established in the Wechsler-Reya lab; MED211FH, MED511FH, and MED1712FH were established in the J. Olson lab at Fred Hutchinson Cancer Research Center;¹ BT-084 was created in the T. Milde lab at the German Cancer Research Center (DKFZ) and MB002 was created by Y.J. Cho lab at Oregon Health and Sciences University; all PDXs were maintained in the Wechsler-Reya lab. Functional studies were carried out using D425 Med and MED211 cells maintained in the Eberhart and Raabe labs.² CHLA-01 and CHLA-01R were purchased from the American Type Culture Collection (ATCC; Manassas, VA).

Overexpression of Inc-HLX-2-7 in MB cells. Plasmid cDNA-Inc-HLX-2-7 was constructed by introducing a EcoRI-XhoI fragment containing the Inc-HLX-2-7 cDNA into the same site in pcDNA4. D425 Med and MED211 cells were transfected with pcDNA4-lnc-HLX-2-7 using Lipofectamine 3000 (Thermo Fisher Scientific) according to the manufacturer’s instructions. Cells were collected after transfection for RNA isolation and cell proliferation assay.

Isolation of single cells from orthotopic xenografts. 30 days after of the injection of D425 Med cells and D425 Med cells with Inc-HLX-2-7 deleted into the cerebellums, tumors were harvested and dissociated using a brain tumor dissociation kit (Miltenyi Biotech Inc., Auburn, CA) according to the manufacturer’s protocol. To enrich human cells, mouse cells were depleted from the dissociated tumor cells using a mouse cell depletion kit (Miltenyi Biotech Inc.). The dissociated tumor cells were further sorted using a FACSAria (Beckton Dickinson, Franklin Lakes, NJ) to obtain live and singlet cells. The cells were resuspended in DPBS with 0.04% BSA to a final concentration of 1×10⁶ cells per ml.

Single-cell RNA-seq library construction and sequencing. Cell suspensions required for generating 8000 single cell gel beads in emulsion (GEM) were loaded onto the Chromium controller (10X Genomics, Pleasanton, CA). Each sample was loaded onto the single cell 3′ v3.1 chip. The 3′ gene expression library was prepared using a Chromium v3.1 single cell 3′ library kit (10X Genomics). The quantification and quality of final libraries were determined using a KAPA PCR (Kapa Biosystems) and a high sensitivity DNA chip (Agilent Technologies), respectively. Samples were diluted to 1.8 pM for loading onto the NextSeq 550 (Illumina) with a 150-cycle paired-end kit using the following read length: 28 cycles for Read 1, 8 cycles i7 index, 0 cycles i5 index, and 91 cycles Read 2.

Processing of scRNA-seq data. Single-cell RNA-seq samples were classified into host and graft reads using XenoCell³ and Xenome v1.0.1⁴. The proportions of graft and host reads were 92.25% and 0.43% for D425, and 86.54% and 2.96% for Inc-HLX-2-7-deleted D425, respectively. The remaining reads were classified as both, neither, or ambiguous. FASTQ files for grafts were aligned to human genome hg38, indexed with GENCODE human annotations v34,⁵ and augmented with IncRNA annotations from LNCipedia v5.2⁶ using 10X Genomics cellranger count (https://support.10xgenomics.com/) and STAR v 2.7.0d_0221.⁷ For downstream integrated analysis, both samples were combined and normalized for the number of mapped reads per cell across libraries using the 10X Genomics cellranger aggr function. 5,547 and 10,039 cells were detected for D425 and Inc-HLX-2-7-deleted D425 cells, respectively, with a post-normalization mean number of 18,034 reads per cell and median of 960 genes detected per cell.

Quality control and clustering analysis of scRNA-seq data. Quality control and clustering of scRNA-seq data were performed using Seurat v3.1.2⁸ in R v3.6.1. Low quality and doublet cells were filtered by selecting cells with <10% mitochondrial percentage (distributions of mitochondrial read percentage for each scRNA-seq sample, before and after filtering, are shown in FIGS. 17) and expressing 200-2500 genes. 3,442 and 6,193 cells were retained for D425 Med and Inc-HLX-2-7-deleted-xenograft samples after filtering. The count matrices for D425 and Inc-HLX-2-7-deleted xenografts were normalized and integrated using the FindIntegrationAnchors and IntegrateData functions. Principle component analysis (PCA) was subsequently performed. For combined clustering, 17 PCs with resolution=0.5 were used to obtain 5 clusters. The marker genes associated with each cluster were identified by finding differentially expressed features across clusters and using log2 fold change cutoff of ±0.2 and adjusted p-value of 0.05. Pathway analysis was performed using IPA.

scRNA-seq cell trajectory analysis. Pseudotemporal trajectory analysis was performed using Monocle3 v0.2.2.⁹ The integrated raw count matrices for D425 and Inc-HLX-2-7-deleted xenografts were converted to Monocle3 CDS format followed by preprocessing. The UMAP space from Seurat analysis was used as input of the reduced dimensional space for pseudotemporal analysis. Graph autocorrelation analysis using the graph_test function was used to find genes that varied over a selected trajectory from cluster 1 (D425 exclusive) to cluster 5 (Inc-HLX-2-7 deleted exclusive) (FIG. 18). 370 genes with Moran’s I values over the threshold of two standard deviations above the median were selected.

### Supplemental References

1. Brabetz S, Leary SES, Gröbner SN, et al. A biobank of patient-derived pediatric brain tumor models. Nat Med. 2018; 24(11):1752-1761.

2. Hanaford AR, Alt J, Rais R, et al. Orally bioavailable glutamine antagonist prodrug JHU-083 penetrates mouse brain and suppresses the growth of MYC-driven medulloblastoma. Transl Oncol. 2019; 12(10):1314-1322.

3. Cheloni S, Hillje R, Luzi L, Pelicci PG, Gatti E. XenoCell: classification of cellular barcodes in single cell experiments from xenograft samples. bioRxiv. 2019.

4. Conway T, Wazny J, Bromage A, et al. Xenome--a tool for classifying reads from xenograft samples. Bioinformatics. 2012; 28(12):i172-178.

5. Frankish A, Diekhans M, Ferreira AM, et al. GENCODE reference annotation for the human and mouse genomes. Nucleic Acids Res. 2019; 47(D1):D766-D773.

6. Volders P-J, Anckaert J, Verheggen K, et al. LNCipedia 5: towards a reference set of human long non-coding RNAs. Nucleic Acids Res. 2019; 47(D1):D135-D139.

7. Dobin A, Davis CA, Schlesinger F, et al. STAR: ultrafast universal RNA-seq aligner. Bioinformatics. 2013; 29(1):15-21.

8. Butler A, Hoffman P, Smibert P, Papalexi E, Satija R. Integrating single-cell transcriptomic data across different conditions, technologies, and species. Nat Biotechnol. 2018; 36(5):411-420.

9. Trapnell C, Cacchiarelli D, Grimsby J, et al. The dynamics and regulators of cell fate decisions are revealed by pseudotemporal ordering of single cells. Nat Biotechnol. 2014; 32(4):381-386.

TABLE 1

IncHLX-2-7 Probes

| ACCESSION | NAME | FUNCTION | PROBE REGION | SEQUENCE | SEQ ID NO |
|---|---|---|---|---|---|
| GS10906 | Inc_HLX-2_71-1R | LE | 19-39 bp | ccagacataaactcgccaagc | 3 |
| GS10906 | Inc_HLX-2_72-IR | LE | 40-57 bp | ccgcatgtgtccaggcac | 4 |
| GS10906 | Inc_HLX-2_73-IR | BL | 58-83 bp | gtgtgtgtgtgtgtgtgtgtttatte | 5 |
| GS10906 | Inc_HLX-2 _74-IR | BL | 84-108 bp | geagtaacatcgaatgtgtatgtet | 6 |
| GS10906 | Inc_HLX-2 _75-IR | BL | 109-132 bp | aaaaaaatgaatggatgaaggaat | 7 |
| GS10906 | Inc_HLX-2 _76-IR | LE | 133-155 bp | ggatccccataaatactgcaatg | 8 |
| GS10906 | Inc_HLX-2_77-IR | LE | 156-179 bp | tctctatggtgcaaacactcacaa | 9 |
| GS10906 | Inc_HLX-2_78-IR | LE | 180-206 bp | tggttaatgcttagtcaaatacttttt | 10 |
| GS10906 | Inc_HLX-2_79-IR | LE | 207-231 bp | acaaacactgatctcttagctggaa | 11 |
| GS10906 | Inc_HLX-2_710-1R | LE | 232-255 bp | caaccacctcagctattttgaatg | 12 |
| GS10906 | Inc_HLX-2_711-1R | LE | 256-277 bp | gggaatactggctgtcctctcc | 13 |
| GS10906 | Inc_HLX-2_712-1R | LE | 278-303 bp | cctaggaaactaataaacctcttttg | 14 |
| GS10906 | Inc_HLX-2_713-1R | LE | 304-327 bp | gtctgtgtcaatttgtgacagcag | 15 |
| GS10906 | Inc_HLX-2_714-1R | LE | 328-355 bp | acacatttctgttgttttaagtcactaa | 16 |
| GS10906 | Inc_HLX-2_715-1R | LE | 356-381 bp | gagaagcagagaatgtagtgacacaa | 17 |
| GS10906 | Inc_HLX-2_716-1R | LE | 382-403 bp | cagaagaagagtccatgtgcca | 18 |
| GS10906 | Inc_HLX-2_717-1R | LE | 404-426 bp | ggaagacagaggaaaactggatg | 19 |
| GS10906 | Inc_HLX-2_718-1R | LE | 427-448 bp | taccacacgcatgcttatgaga | 20 |
| GS10906 | Inc_HLX-2_719-1R | LE | 449-468 bp | cctgggtggacgctaaatgc | 21 |

LE: Label extenders; CE: Capture oligos; BL: Blocker oligos.

TABLE 2

SPRY4-IT1 Probes

| ACCESSION | NAME | FUNCTION | PROBE REGION | SEQUENCE | SEQ ID NO |
|---|---|---|---|---|---|
| NR_131221 | SPRY4-IT11-4R | LE | 45-66 bp | ggcagatcacttgaggtcagga | 22 |
| NR_131221 | SPRY4-IT12-4R | LE | 67-86 bp | ccttttgggaggccaaggta | 23 |
| NR_131221 | SPRY4-IT13-4R | LE | 87-108 bp | tggcteatgectgtaatetcag | 24 |
| NR_131221 | SPRY4-IT14-4R | LE | 109-126 bp | aaagaaggeetggegeag | 25 |
| NR_131221 | SPRY4-IT15-4R | BL | 127-153 bp | aaaaaaaaagaaagaaaaaaagaaaag | 26 |
| NR_131221 | SPRY4-IT16-4R | LE | 154-177 bp | cagcacagctaaatgatgtctcaa | 27 |
| NR_131221 | SPRY4-IT17-4R | LE | 178-199 bp | agctgcctatttaagaacccct | 28 |
| NR_131221 | SPRY4-IT18-4R | LE | 200-224 bp | gctgacaaaggaaaacaattttctg | 29 |
| NR_131221 | SPRY4-IT19-4R | LE | 225-248 bp | aagagcctctgctgaatttatgtg | 30 |
| NR_131221 | SPRY4-IT110-4R | LE | 249-266 bp | caccagcagggaccctcc | 31 |
| NR_131221 | SPRY4-IT111-4R | LE | 267-284 bp | actgctggcctcacccct | 32 |
| NR_131221 | SPRY4-IT112-4R | LE | 285-307 bp | agcaaaaaccaaatcagagttcc | 33 |
| NR_131221 | SPRY4-IT113-4R | LE | 308-329 bp | gattcctttcaaccaccagctc | 34 |
| NR_131221 | SPRY4-IT114-4R | LE | 330-354 bp | ccctattataaccccgatgtagtag | 35 |
| NR_131221 | SPRY4-IT115-4R | LE | 355-380 bp | actgggcatattctaaaatgtatctt | 36 |
| NR_131221 | SPRY4-IT116-4R | LE | 381-398 bp | gcagcatccgatggctcc | 37 |
| NR_131221 | SPRY4-IT117-4R | LE | 399-417 bp | ttggetetetggggaegat | 38 |
| NR_131221 | SPRY4-IT118-4R | LE | 418-436 bp | gagcttggcccacgatgac | 39 |
| NR_131221 | SPRY4-IT119-4R | LE | 437-454 bp | ggccagacatggggatgg | 40 |
| NR_131221 | SPRY4-IT120-4R | LE | 455-473 bp | catctgggcctgcagttga | 41 |
| NR_131221 | SPRY4-IT121-4R | LE | 474-493 bp | cctccagaggcagctgtcaa | 42 |
| NR_131221 | SPRY4-IT122-4R | LE | 494-514 bp | gcattcacaggctcccataac | 43 |
| NR_131221 | SPRY4-IT123-4R | LE | 515-533 bp | gcaggcaatggggatgttg | 44 |
| NR_131221 | SPRY4-IT124-4R | LE | 534-550 bp | ggatgggagcagccgct | 45 |
| NR_131221 | SPRY4-IT125-4R | LE | 551-569 bp | aagtcccaccaggaagcca | 46 |
| NR_131221 | SPRY4-IT126-4R | LE | 570-590 bp | cagattccccaattcatggaa | 47 |
| NR_131221 | SPRY4-IT127-4R | LE | 591-613 bp | taataggeettggaateagaaag | 48 |
| NR_131221 | SPRY4-IT128-4R | LE | 614-634 bp | atgggcaatgctcagaaattt | 49 |
| NR_131221 | SPRY4-IT129-4R | LE | 635-660 bp | catgtcctacagataaagcaaaagaa | 50 |

TABLE 3

MYC Probes

| ACCESSION | NAME | FUNCTION | PROBE REGION | SEQUENCE | SEQ ID NO |
|---|---|---|---|---|---|
| NM_002467 | MYC1-6R | LE | 566-583 bp | cgttgaggggcategtcg | 51 |
| NM_002467 | MYC2-6R | LE | 584-607 bp | catagttcctgttggtgaagctaa | 52 |
| NM_002467 | MYC3-6R | LE | 608-628 bp | gcaccgagtcgtagtcgaggt | 53 |
| NM_002467 | MYC4-6R | LE | 629-649 bp | cgtcgcagtagaaatacggct | 54 |
| NM_002467 | MYC5-6R | LE | 650-672 bp | ctgctggtagaagttctcctcct | 55 |
| NM_002467 | MYC6-6R | LE | 673-691 bp | gcagctcgctctgctgctg | 56 |
| NM_002467 | MYC7-6R | BL | 692-704 bp | ggcgccgggggct | 57 |
| NM_002467 | MYC8-6R | BL | 705-726 bp | tttcttccagatatcctcgctg | 58 |
| NM_002467 | MYC9-6R | LE | 727-744 bp | ggtgggcagcagctcgaa | 59 |
| NM_002467 | MYC10-6R | LE | 745-761 bp | ctaggggacaggggcgg | 60 |
| NM_002467 | MYC11-6R | BL | 762-774 bp | cccggagcggcgg | 61 |
| NM_002467 | MYC12-6R | LE | 775-793 bp | cgtaggagggcgagcagag | 62 |
| NM_002467 | MYC13-6R | LE | 794-813 bp | ggagaagggtgtgaccgcaa | 63 |
| NM_002467 | MYC14-6R | BL | 814-832 bp | cgtcgttgtctccccgaag | 64 |
| NM_002467 | MYC17-6R | BL | 865-884 bp | agctcggtcaccatctccag | 65 |
| NM_002467 | MYC18-6R | LE | 885-904 bp | tcaccatgtctcctcccagc | 66 |
| NM_002467 | MYC19-6R | LE | 905-925 bp | ggtcgcagatgaaactctggt | 67 |
| NM_002467 | MYC20-6R | LE | 926-945 bp | gatgaaggtetegtegteeg | 68 |
| NM_002467 | MYC21-6R | LE | 946-969 bp | acagtcctggatgatgatgttttt | 69 |
| NM_002467 | MYC22-6R | BL | 970-988 bp | ccgagaagccgctccacat | 70 |
| NM_002467 | MYC30-6R | BL | 1111-1124 bp | gcggcggcgctcag | 71 |
| NM_002467 | MYC31-6R | LE | 1125-1144 bp | aggggtcgatgcactctgag | 72 |
| NM_002467 | MYC32-6R | LE | 1145-1163 bp | gggtagggpaagaccaccg | 73 |
| NM_002467 | MYC33-6R | BL | 1164-1183 bp | gcgagctgctgtcgttgaga | 74 |
| NM_002467 | MYC34-6R | LE | 1184-1200 bp | cgaggcgcaggacttgg | 75 |
| NM_002467 | MYC35-6R | LE | 1201-1220 bp | gagaaggcgctggagtcttg | 76 |
| NM_002467 | MYC36-6R | LE | 1221-1240 bp | gcagagaatccgaggacgga | 77 |
| NM_002467 | MYC37-6R | LE | 1241-1260 bp | ggaggactccgtcgaggaga | 78 |
| NM_002467 | MYC38-6R | BL | 1261-1275 bp | ggggctgccctgcgg | 79 |
| NM_002467 | MYC39-6R | BL | 1276-1293 bp | atggagcaccaggggctc | 80 |
| NM_002467 | MYC40-6R | LE | 1294-1311 bp | ggtgggcggtgtctcctc | 81 |
| NM_002467 | MYC41-6R | LE | 1312-1331 bp | tcctcagagtcgctgctggt | 82 |
| NM_002467 | MYC42-6R | LE | 1332-1356 bp | gatttcttcctcatcttcttgttcc | 83 |
| NM_002467 | MYC43-6R | LE | 1357-1380 bp | cctcttttccacagaaacaacatc | 84 |
| NM_002467 | MYC44-6R | LE | 1381-1399 bp | accttttgccaggagcctg | 85 |
| NM_002467 | MYC45-6R | LE | 1400-1422 bp | agcagaaggtgatccagactctg | 86 |
| NM_002467 | MYC46-6R | LE | 1423-1441 bp | gaggtttgctgtggcctcc | 87 |
| NM_002467 | MYC47-6R | LE | 1442-1461 bp | gaggaccagtgggctgtgag | 88 |
| NM 002467 | MYC48-6R | LE | 1462-1481 bp | gtggagacgtggcacctctt | 89 |
| NM 002467 | MYC49-6R | LE | 1482-1502 bp | gctgcgtagttgtgctgatgt | 90 |
| NM 002467 | MYC50-6R | LE | 1503-1520 bp | ttccgagtggagggaggc | 91 |
| NM 002467 | MYC51-6R | LE | 1521-1541 bp | ctcttggcagcaggatagtcc | 92 |
| NM 002467 | MYC52-6R | LE | 1542-1565 bp | actctgacactgtccaacttgacc | 93 |
| NM 002467 | MYC53-6R | LE | 1566-1588 bp | ggttgttgctgatctgtctcagg | 94 |
| NM 002467 | MYC54-6R | LE | 1589-1606 bp | tggggctggtgcattttc | 95 |
| NM 002467 | MYC55-6R | LE | 1607-1624 bp | cctcggtgtccgaggacc | 96 |
| NM 002467 | MYC56-6R | LE | 1625-1646 bp | tgtgttcgcctcttgacattct | 97 |
| NM 002467 | MYC57-6R | LE | 1647-1664 bp | tggcgctccaagacgttg | 98 |
| NM 002467 | MYC58-6R | BL | 1665-1686 bp | ccgttttagctcgttcctcctc | 99 |
| NM 002467 | MYC62-6R | BL | 1744-1768 bp | tggcttttttaaggataactacctt | 100 |
| NM 002467 | MYC63-6R | LE | 1769-1789 bp | ggacggacaggatgtatgctg | 101 |
| NM 002467 | MYC64-6R | LE | 1790-1810 bp | tgagcttttgctcctctgctt | 102 |

TABLE 4

MYCN Probes

| ACCESSION | NAME | FUNCTION | PROBE REGION | SEQUENCE | SEQ ID NO |
|---|---|---|---|---|---|
| NM_005378 | MYCN1-6R | LE | 1357-1375 bp | gctcgctggactgagccct | 103 |
| NM_005378 | MYCN2-6R | LE | 1376-1396 bp | gaaggcatcgtttgaggatca | 104 |
| NM_005378 | MYCN3-6R | BL | 1397-1415 bp | ttgtgctgctggtggatgg | 105 |
| NM_005378 | MYCN6-6R | BL | 1456-1478 bp | ctctttatcttcttctgtggggg | 106 |
| NM_005378 | MYCN7-6R | LE | 1479-1494 bp | acgtggggacgcctcg | 107 |
| NM_005378 | MYCN8-6R | LE | 1495-1514 bp | gggatgacactcttgagcgg | 108 |
| NM_005378 | MYCN9-6R | BL | 1515-1535 bp | ctcaagctcttagcctttggg | 109 |
| NM_005378 | MYCN10-6R | BL | 1536-1554 bp | cgagtcagagtttcggggg | 110 |
| NM_005378 | MYCN11-6R | LE | 1555-1573 bp | tgcgacgctcactgtcctc | 111 |
| NM_005378 | MYCN12-6R | LE | 1574-1594 bp | gctccaggatgttgtggtttc | 112 |
| NM_005378 | MYCN13-6R | BL | 1595-1609 bp | cgttgcggcgctggc | 113 |
| NM_005378 | MYCN14-6R | LE | 1610-1630 bp | tgagaaagctggaccgaaggt | 114 |
| NM_005378 | MYCN15-6R | LE | 1631-1648 bp | gcacgtggtccctgagcg | 115 |
| NM_005378 | MYCN16-6R | LE | 1649-1672 bp | ccttctcattctttaccaactccg | 116 |
| NM_005378 | MYCN17-6R | LE | 1673-1691 bp | aaaatgaccaccttggcgg | 117 |
| NM_005378 | MYCN18-6R | LE | 1692-1714 bp | ggacatactcagtggccttttic | 118 |
| NM_005378 | MYCN19-6R | LE | 1715-1732 bp | cctcggcctggagggagt | 119 |
| NM_005378 | MYCN20-6R | LE | 1733-1752 bp | ttccagcaaaagctggtgct | 120 |
| NM_005378 | MYCN21-6R | LE | 1753-1773 bp | tcttgcctgcaatttttcctt | 121 |
| NM_005378 | MYCN22-6R | LE | 1774-1796 bp | attttctttagcaactgctgctg | 122 |
| NM_005378 | MYCN23-6R | LE | 1797-1815 bp | gcaagtccgagcgtgttca | 123 |
| NM_005378 | MYCN24-6R | LE | 1816-1838 bp | tgtccagttttgagaagcgtcta | 124 |
| NM_005378 | MYCN25-6R | LE | 1839-1860 bp | aaatgtgcaaagtggcagtgac | 125 |
| NM_005378 | MYCN26-6R | BL | 1861-1887 bp | cacaatgtttgtttaaaaaaaaaatca | 126 |
| NM_005378 | MYCN27-6R | LE | 1888-1914 bp | aaagtaaaccaacattcttaatgtcaa | 127 |
| NM_005378 | MYCN28-6R | LE | 1915-1933 bp | tcgacaggggaccgatttg | 128 |
| NM_005378 | MYCN29-6R | LE | 1934-1951 bp | gcccacccagagccgaac | 129 |
| NM_005378 | MYCN30-6R | LE | 1952-1972 bp | ccccacactggtggtectact | 130 |
| NM_005378 | MYCN31-6R | BL | 1973-1992 bp | tctccaaggtcccagcagaa | 131 |
| NM_005378 | MYCN34-6R | BL | 2027-2047 bp | catggaggtgaggtggaggag | 132 |
| NM_005378 | MYCN35-6R | LE | 2048-2067 bp | tcaccaacgtttagcgctgt | 133 |
| NM_005378 | MYCN36-6R | LE | 2068-2085 bp | cccagaggctcccaaccg | 134 |
| NM_005378 | MYCN37-6R | LE | 2086-2108 bp | acacacaaggtgacttcaacagc | 135 |
| NM_005378 | MYCN38-6R | LE | 2109-2131 bp | tttctgttgtttggaaacttgga | 136 |
| NM_005378 | MYCN39-6R | LE | 2132-2156 bp | caccattttaaaaagaaggaatgac | 137 |
| NM_005378 | MYCN40-6R | LE | 2157-2178 bp | gtggeatetgetggaaettaag | 138 |
| NM_005378 | MYCN41-6R | LE | 2179-2200 bp | tatcaaatggcaaaccccttat | 139 |
| NM_005378 | MYCN42-6R | LE | 2201-2219 bp | cagaaatgttccccagggg | 140 |
| NM 005378 | MYCN43-6R | LE | 2220-2242 bp | ggcggatgtgtcaatggtattta | 141 |
| NM 005378 | MYCN44-6R | LE | 2243-2268 bp | tctcattacccaggatgtatacaaaa | 142 |
| NM 005378 | MYCN45-6R | LE | 2269-2284 bp | ggccgcaaaagccacc | 143 |
| NM 005378 | MYCN46-6R | LE | 2285-2313 bp | acttaggtatgaacttccagtctaatact | 144 |
| NM 005378 | MYCN47-6R | BL | 2314-2340 bp | cctcaaacattgaggtattattacagt | 145 |
| NM 005378 | MYCN54-6R | BL | 2518-2552 bp | catatatatatagtaaatttctttacaaaagtttc | 146 |
| NM 005378 | MYCN55-6R | LE | 2553-2575 bp | gaagaaacaggctaggaaaaagg | 147 |
| NM 005378 | MYCN56-6R | LE | 2576-2601 bp | ccaaacatgaacaaatacattaacag | 148 |
| NM 005378 | MYCN57-6R | LE | 2602-2624 bp | ttgcatttacccagttctatgca | 149 |
| NM 005378 | MYCN58-6R | LE | 2625-2651 bp | cattttgaagaaattaaacacagaact | 150 |
| NM 005378 | MYCN59-6R | LE | 2652-2679 bp | tgctataagatgcagcactaaatatata | 151 |
| NM 005378 | MYCN60-6R | LE | 2680-2706 bp | ttttcataaacatgaggtatttcaaag | 152 |

TABLE 5

MALAT Probes

| ACCESSION | NAME | FUNCTIO N | PROBE REGION | SEQUENCE | SEQ ID NO |
|---|---|---|---|---|---|
| NR_002819 | MALAT11-4R | LE | 4056-4078 bp | caggctggttatgactcagaaga | 153 |
| NR_002819 | MALAT12-4R | LE | 4079-4100 bp | tgcatctaggccatcatactgc | 154 |
| NR_002819 | MALAT13-4R | LE | 4101-4123 bp | attcaccaaggagctgttttctc | 155 |
| NR_002819 | MALAT14-4R | LE | 4124-4151 bp | atataatcttttctgcctttacttatca | 156 |
| NR_002819 | MALAT15-4R | LE | 4152-4174 bp | ttattccccaatggaggtatgac | 157 |
| NR_002819 | MALAT16-4R | LE | 4175-4200 bp | cagtagtaagaatctcagggttatgc | 158 |
| NR_002819 | MALAT17-4R | LE | 4201-4225 bp | tggcatatgcagataatgttctcat | 159 |
| NR_002819 | MALAT18-4R | LE | 4226-4250 bp | tagctttcatttgcttaaaattttt | 160 |
| NR_002819 | MALAT19-4R | LE | 4251-4275 bp | ggtagattccgtaactttaaattgg | 161 |
| NR_002819 | MALAT110-4R | LE | 4276-4301 bp | gcttgacaagcaattaactttaaaat | 162 |
| NR_002819 | MALAT111-4R | LE | 4302-4328 bp | catcaattcattatttttgtggttata | 163 |
| NR_002819 | MALAT112-4R | LE | 4329-4353 bp | gacattgcctcttcattgtatttct | 164 |
| NR_002819 | MALAT113-4R | LE | 4354-4379 bp | ttttgtaaaagcagtattttgagatg | 165 |
| NR_002819 | MALAT114-4R | LE | 4380-4403 bp | catttcttttcgcttttattctgc | 166 |
| NR_002819 | MALAT115-4R | LE | 4404-4430 bp | tccaggattaatgtagtgtaacatttt | 167 |
| NR_002819 | MALAT116-4R | LE | 4431-4455 bp | tctcatttatttcggcttcttttat | 168 |
| NR_002819 | MALAT117-4R | LE | 4456-4478 bp | aatccacttgatcccaactcatc | 169 |
| NR_002819 | MALAT118-4R | LE | 4479-4498 bp | gcacacagcacagcctcctc | 170 |
| NR_002819 | MALAT119-4R | BL | 4499-4520 bp | gtctgaggcaaacgaaacattg | 171 |
| NR_002819 | MALAT120-4R | LE | 4521-4547 bp | aactcttctgataacgaagagatacct | 172 |
| NR_002819 | MALAT121-4R | LE | 4548-4568 bp | tgctcccagatgaaatgaagc | 173 |
| NR_002819 | MALAT122-4R | BL | 4569-4590 bp | ttaacagctgcctgctgttttc | 174 |
| NR_002819 | MALAT123-4R | BL | 4591-4615 bp | tgcagatgcaagttaaacttatctg | 175 |
| NR_002819 | MALAT124-4R | LE | 4616-4640 bp | agcacttatccctaacatgcaatac | 176 |
| NR_002819 | MALAT125-4R | LE | 4641-4667 bp | ttaagaactccacagctcttaaaaata | 177 |
| NR_002819 | MALAT126-4R | BL | 4668-4690 bp | ggagaaagtgccatggttgatat | 178 |
| NR_002819 | MALAT127-4R | BL | 4691-4709 bp | tcccctagggaaggggtca | 179 |
| NR_002819 | MALAT128-4R | LE | 4710-4733 bp | tggaaaaatttctcaatcctgaaa | 180 |
| NR_002819 | MALAT129-4R | LE | 4734-4756 bp | cctacaattttaaaaaggctcga | 181 |
| NR_002819 | MALAT130-4R | LE | 4757-4777 bp | ctgaagcccacaggaacaagt | 182 |
| NR_002819 | MALAT131-4R | LE | 4778-4803 bp | tctgagtgaagtgtactatcccatca | 183 |
| NR_002819 | MALAT132-4R | LE | 4804-4828 bp | gaaattatttaaagatgcaaatgcc | 184 |
| NR_002819 | MALAT133-4R | LE | 4829-4854 bp | gcactgatcactttagaggcttttaa | 185 |
| NR_002819 | MALAT134-4R | LE | 4855-4878 bp | caaatttccttagttggcatcaag | 186 |
| NR_002819 | MALAT135-4R | LE | 4879-4901 bp | gccttcagagattcaatgctaaa | 187 |
| NR_002819 | MALAT136-4R | LE | 4902-4926 bp | cacatcatgctattcctttcataga | 188 |
| NR_002819 | MALAT137-4R | LE | 4927-4954 bp | ttttagcagtaacatctgattctaacag | 189 |
| NR_002819 | MALAT138-4R | LE | 4955-4982 bp | ctacacaatttacatcacaacatgtaaa | 190 |
| NR_002819 | MALAT139-4R | LE | 4983-5010 bp | ttattattttgaatgatttaatggtttt | 191 |
| NR_002819 | MALAT140-4R | BL | 5011-5043 bp | ttctaaaagtatacattctctaataaaaatagt | 192 |
| NR_002819 | MALAT141-4R | LE | 5044-5072 bp | cactattttatttaaataaggagacagct | 193 |
| NR_002819 | MALAT142-4R | LE | 5073-5096 bp | ccccaacactgaactacagacaaa | 194 |
| NR_002819 | MALAT143-4R | BL | 5097-5116 bp | aagaatcccccccaagattg | 195 |
| NR_002819 | MALAT144-4R | LE | 5117-5143 bp | gcagacaaagtttctgaaagattagag | 196 |
| NR_002819 | MALAT145-4R | LE | 5144-5168 bp | tgatctggtccattaaagagtgttc | 197 |
| NR_002819 | MALAT146-4R | LE | 5169-5188 bp | tcgttcttccgctcaaatcc | 198 |
| NR_002819 | MALAT147-4R | LE | 5189-5213 bp | tgtctttcctgccttaaagttacat | 199 |

TABLE 6

17-lncRNAs Associated with Prognostic Signature in MB

| Gene Name | Penalized Coefficient |
|---|---|
| Inc-TMEM258-3 | -0.47771 |
| ZNRF3-ASI | -0.24098 |
| Inc-TMEM121-3 | -0.17041 |
| MAP3K14-AS1 | -0.07358 |
| LINC01152 | -0.0675 |
| KLF3-ASI | -0.05371 |
| Inc-PRR34-1 | -0.0379 |
| Inc-FOXD4L5-25 | -0.03664 |
| AC209154.1 | -0.01405 |
| TTC28-ASI | -0.00891 |
| FAM222A-AS1 | 0.07403 |
| LINC00336 | 0.042296 |
| LINC01551 | 0.073539 |
| H19 | 0.102589 |
| lnc-RRM2-3 | 0.107783 |
| lnc-CDYL-1 | 0.198379 |
| AL139393.2 | 0.231787 |

17-lncRNAs identified from penalized COX regression analysis of Cavalli17 dataset. Negative coefficient values highlights good prognosis marker candidates and positive coefficient value highlights bad prognosis marker candidates.

TABLE 7

List of MB Cases with Clinical Features Analyzed in RNA-FISH

| Tissue Diagnosis ID | Subgroup | Histology | Age | Overall Survival to Sex Last Visit | |
|---|---|---|---|---|---|
| 18828 | SHH | Nodular MB | 9 | M | 16 |
| 18830 | Group 4 | Classic MB | 12 | M | 37 |
| 18831 | SHH | Nodular MB | 3 | M | 72 |
| 18834 | SHH | Nodular MB | 20 | M | 200 |
| 18837 | SHH | Nodular MB | 6 | M | 25 |
| 18838 | SHH | Nodular MB | 2 | F | 215 |
| 18840 | SHH | Nodular MB | 12 | F | 34 |
| 18841 | Group 4 | Nod MB with Anap | 2 | M | 21 |
| 18842 | Unknown | MB | 11 | F | 101 |
| 18843 | Group 3 | Mod A | 12 | F | Unknown |
| 18844 | Unknown | Classic MB | 5 | F | 8 |
| 18845 | Unknown | Classic MB | 16 | F | 58 |
| 18846 | SHH | Sev A | | M | 28 |
| 18847 | SHH | Nodular MB | 1 | M | Unknown |
| 18850 | Unknown | PNET/Pineoblastoma | 21 | F | 46 |
| 18851 | Group 3 | LCMB | 9 | M | 9 |
| 18852 | SHH | Classic MB | 22 | M | Unknown |
| 18853 | Unknown | AT/RT | 8 | M | 1M |
| 18854 | Unknown | AT/RT | 8M | F | 4 |
| 18855 | Unknown | AT/RT | 11M | M | 1M |
| 18856 | SHH | Nodular MB | 11 | M | 109 |
| 18857 | Unknown | Medulloblastoma | 11 | F | Unknown |
| 18858 | Unknown | PNET | 3 | F | 1M |
| 18859 | Group 4 | MB | 2 | F | 27 |
| 18860 | Unknown | Anaplastic MB | 8 | F | 27 |
| 18861 | SHH | Classic MB | 8 | F | 123 |
| 18862 | Group 4 | MB | 10 | Unknown | Unknown |
| 18863 | Group 4 | MB | 5 | M | 198 |
| 18864 | SHH | Desmoplastic MB | 7 | M | 119 |
| 18865 | Group 4 | F Mod A | 10 | M | 119 |
| 18866 | Group 4 | Classic MB | 9 | M | 103 |
| 18867 | Unknown | Mod A | 18 | M | 84 |
| 18868 | Group 4 | Classic MB | 15 | M | 118 |
| 18869 | Group 4 | MB | 13 | F | 69 |
| 18870 | Group 3 | LCMB | 5 | M | 10 |
| 18871 | Group 4 | Classic MB | 13 | F | 121 |
| 18872 | SHH | Nodular MB | 11M | M | 39 |
| 18873 | SHH | Nodular MB | 38 | F | 207 |
| 18874 | Unknown | Mod A | 9 | M | 170 |
| 18875 | Unknown | Medulloepithelioma | 1 | F | 1 |
| 18876 | Unknown | Medulloepithelioma | 1 | F | 19M |
| 18877 | SHH | MB | 15 | F | 63 |
| 18878 | unknown | Classic MB | 6 | F | 18 |
| 18879 | SHH | Classic MB | 38 | M | 32M |
| 18880 | Group 4 | F Mod A | 5 | F | 35 |
| 18881 | SHH | Classic MB | 6 | M | 127 |
| 18882 | Group 3 | Classic MB | 1 | M | 1M |
| 18883 | SHH | MB with desmoplasia | 16 | F | 167 |
| 18884 | SHH | Sev A with Nodules | | M | 12 |
| 18885 | SHH | Classic MB | 3 | M | 100 |
| 18886 | Group 4 | Sev A | 6 | F | 147 |
| 18887 | Group 4 | Sev A | | M | 96 |
| 18888 | Group 3 | Sev A | 18 | M | 12 |
| 18890 | SHH | LCMB | 2 | M | 47 |
| 18891 | Group 4 | F Mod A | 6 | F | 37 |
| 18892 | Group 3 | Sev A | 12 | M | 23 |
| 18893 | SHH | Classic MB | 38 | M | 12 |
| 18894 | SHH | F Mod A | 16 | M | 20 |
| 18895 | SHH | Mod A | | F | 12 |
| 18896 | Unknown | F Mod A | 9 | F | 101 |
| 18897 | Group 3 | F Mod A | 10 | M | Unknown |
| 18898 | SHH | Nodular MB | 29 | F | 28 |
| 18899 | Unknown | Classic MB | 12 | F | 183 |
| 18900 | Unknown | PNET | 10 | F | 55 |
| 18901 | SHH | Mod A | 31 | F | 10 |
| 18902 | Group 3 | Unknown | 4 | M | 12 |
| 18903 | Unknown | PNET | 35 | M | 20 |
| 18904 | unknown | MB met to mandible | 9 | M | 19 |
| 18905 | WNT | Mod A | 9 | F | 187 |
| 18906 | SHH | Classic MB | 2 | F | 120 |
| 18907 | Group 4 | Classic MB | 8 | F | 31 |
| 56510 | Unknown | MB | 11 | F | 101 |
| 61379 | Group 3 | Sev A | 11 | M | 27 |
| 61380 | Group 4 | Mod A | 32 | F | 60 |
| 61382 | SHH | Unknown | 55 | F | 9 |
| 61383 | SHH | Nodular MB/MBEN | 1 | M | Unknown |
| 61384 | Unknown | MB | 16 | M | 147 |
| 61386 | Group 4 | Nodular MB | 28 | M | 25 |
| 61387 | Unknown | Medulloepithelioma | 5 | M | 22 |
| 61403 | Unknown | PNET | 1 | M | 34 |

TABLE 8

Primer sequences for qRT-PCR

| Target gene | Primer sequence (5′ to 3′) | SEQ ID NO. |
|---|---|---|
| ACTB | Forward: cctggcattgccgacaggatg | 204 |
| | Reverse: ccgatccacacggagtacttgcg | 205 |
| lnc-HLX-2-7 | Forward: gcttctctggcacatggact | 206 |
| | Reverse: gtccttcgtgagcacagcat | 207 |
| HLX | Forward: gcttctctggcacatggact | 208 |
| | Reverse: gtccttcgtgagcacagcat | 209 |
| MYC | Forward: aaaggcccccaaggtagtta | 210 |
| | Reverse: gcacaagagttccgtagctg | 211 |
| MYCN | Forward: ctaatactggccgcaaaagc | 212 |
| | Reverse: cataaggggtttgccatttg | 213 |
| PTGR1 | Forward: cagacacaataccactgtctttgg .^{.}.^{.}.^{.}.^{.} | 214 |
| | Reverse: ctgcattaaccatcactgtttctc | 215 |
| FZD6 | Forward: agactctctggggaacaggtc | 216 |
| | Reverse: ggccagtgtcagtaatatcactctt | 217 |
| TRPM3 | Forward: aatacttcagagaaaaggatgatcg | 218 |
| | Reverse: gagtgctctctctcgttgacttc | 219 |
| NAMPT | Forward: aaaagggeegattatetttaeatag | 220 |
| | Reverse: ccattcttgaagacagtatggagaa | 221 |
| NRBP2 | Forward: aggacgagagcgacatcct | 222 |
| | Reverse: ggctaggaaggtgctctgaag | 223 |
| NBAT1 | Forward: gtttatccatcttcagctccactct | 224 |
| | Reverse: tctgtgggtttcagtttcttcat | 225 |
| CCNG2 | Forward: caacagctactatagtgttcctgagc | 226 |
| | Reverse: tctcctctccacaactcatatcttc | 227 |
| ELK4 | Forward: gcaagaacaagcctaacatgaatta | 228 |
| | Reverse: acacaaacttctgaccattcacttt | 229 |
| CDKN2C | Forward: ttgcaaaataatgtaaacgtcaatg | 230 |
| | Reverse: ttagcacctctaagtagcagtctcc | 231 |
| CDK6 | Forward: caaccaattgagaagtttgtaacag | 232 |
| | Reverse: ggcactgtaggcagatattctttt | 233 |

TABLE 9

Primer sequences for ChIP-qPCR

| Target gene | Primer sequence (5′ to 3′) | SEQ ID NO. |
|---|---|---|
| HLX-2KB | Forward: ttatttcttaagagagagggtgagg | 234 |
| | Reverse: aatttgactgcaaacatttagacct | 235 |
| HLX-TSS | Forward: tacgcagagtagcaagaagcact | 236 |
| | Reverse: tggaggggaattaggaacaag | 237 |
| E-box | Forward: taataaacaaaaccgcctagatgag | 238 |
| | Reverse: aaaggctttacataaatcggcttac | 239 |

TABLE 10

Top 50 Differentially Upregulated IncRNAs in Group 3 MB

| Gene.ID | Fold Change (log2) | p-value |
|---|---|---|
| lnc-STAP1-13 | 12.3151 | 1.0645E-04 |
| Inc-SLITRK1-1 | 12.2487 | 9.3581E-09 |
| Inc-MYO3A-1 | 11.7062 | 6.4649E-06 |
| lnc-AXIN1-1 | 10.4428 | 6.0770E-05 |
| Inc-POU5F1B-5 | 10.3653 | 1.0766E-16 |
| LINC02342 | 9.9339 | 1.7037E-09 |
| lnc-PDGFA-17 | 9.8875 | 6.7468E-08 |
| Inc-SERPINB3-3 | 9.7623 | 6.1748E-17 |
| Inc-STAP1-2 | 9.6277 | 6.6276E-12 |
| LINC01467 | 9.3207 | 2.0908E-20 |
| lnc-IGLL1-4 | 9.2966 | 3.1889E-05 |
| Inc-HLX-1 | 8.7256 | 7.1143E-22 |
| Inc-SYT1-2 | 8.6991 | 5.2255E-10 |
| Inc-SYK-13 | 8.4236 | 3.7254E-06 |
| lnc-HLX-5 | 7.8451 | 1.2979E-14 |
| lnc-NFATC1-1 | 7.6858 | 7.9406E-06 |
| lnc-APBA2-9 | 7.6460 | 6.4510E-09 |
| lnc-MAGEA12-3 | 7.5857 | 6.3922E-10 |
| LINC02378 | 7.2464 | 2.8975E-06 |
| lnc-PRSS1-1 | 7.0733 | 4.6988E-13 |
| lnc-MGST1-7 | 6.8156 | 8.2139E-12 |
| ESRG | 6.7417 | 1.5129E-36 |
| Inc-KIAA1210-1 | 6.5451 | 2.5660E-06 |
| Inc-PRSS1-7 | 6.5187 | 7.3174E-13 |
| lnc-VCX-6 | 6.4619 | 1.2289E-06 |
| lnc-ANXA1-3 | 6.4276 | 1.3172E-11 |
| lnc-BARD1-1 | 6.4074 | 1.5158E-08 |
| Inc-CSAG3-1 | 6.3320 | 2.2890E-10 |
| Inc-EHF-1 | 6.2428 | 1.4561E-06 |
| lnc-UTP23-12 | 6.2302 | 6.4442E-09 |
| lnc-WRN-6 | 6.1788 | 1.9794E-07 |
| lnc-WRN-5 | 6.1445 | 6.4673E-06 |
| Inc-MYO3A-2 | 6.1383 | 3.6319E-06 |
| Inc-DDX60L-3 | 6.0955 | 2.0418E-07 |
| Inc-HLX-6 | 6.0672 | 1.3604E-23 |
| LINC01501 | 6.0319 | 1.8883E-16 |
| Inc-HLX-2 | 5.9981 | 7.5051E-11 |
| lnc-FRG2C-5 | 5.9857 | 7.8191E-04 |
| Inc-ALX1-2 | 5.9346 | 2.2683E-44 |
| lnc-PLXNA2-3 | 5.9338 | 3.8248E-04 |
| LINC02466 | 5.9253 | 4.1574E-06 |
| Inc-RAB17-1 | 5.8827 | 4.4016E-12 |
| Inc-PLA2G4A-5 | 5.8815 | 3.0865E-04 |
| Inc-CCT8L2-1 | 5.8456 | 1.3576E-04 |
| LINC01323 | 5.8226 | 2.5514E-06 |
| lnc-BMP2-2 | 5.7603 | 8.9155E-06 |
| lnc-WRN-3 | 5.7561 | 2.3437E-09 |
| lnc-RMDN 1-2 | 5.6879 | 2.9128E-05 |
| Inc-SLC22A16-2 | 5.6778 | 1.7633E-32 |
| LINC01324 | 5.6254 | 7.7208E-12 |

That which is claimed:

1. A method for treating medulloblastoma in a patient comprising the step of administering a composition comprising an antisense oligonucleotides (ASO) targeting long noncoding ribonucleic acid HLX-2-7 (lnc-HLX-2-7).

2. The method of claim 1, wherein medulloblastoma is group III medulloblastoma.

3. The method of claim 1, wherein the ASO targets a 20-40 nucleotide sequence of lnc-HLX-2-7 (SEQ ID NO:200).

4. The method of claim 3, wherein the ASO targets nucleotides 325-345 of SEQ ID NO:200.

5. The method of claim 4, wherein the ASO comprises SEQ ID NO:242 or SEQ ID NO:290.

6. The method of claim 3, wherein the ASO targets nucleotides 335-361 of SEQ ID NO:200).

7. The method of claim 6, wherein the ASO comprises SEQ ID NO:247 or SEQ ID NO:292.

8. The method of claim 3, wherein the ASO targets nucleotides 468-488 of SEQ ID NO:200.

9. The method of claim 8, wherein the ASO comprises SEQ ID NO:240 or SEQ ID NO:289.

10. The method of claim 3, wherein the ASO targets nucleotides 480-500 of SEQ ID NO:200.

11. The method of claim 10, wherein the ASO comprises SEQ ID NO:244 or SEQ ID NO:291.

12. The method of claim 19, wherein the composition further comprises a polymeric micelle.

13. The method of claim 12, where in the polymeric micelle comprises cerium oxide nanoparticle.

14. A method comprising the steps of:
(a) detecting overexpression of lnc-HLX-2-7 in a sample obtained from a patient having medulloblastoma; and
(b) treating the patient with a composition comprising a polymeric micelle and an ASO that targets lnc-HLX-2-7.

15. A method comprising the step of administering a composition comprising a polymeric micelle and an ASO that targets lnc-HLX-2-7 to a patient diagnosed with group III medulloblastoma.

16. The method of claim 14, wherein the method further comprises administering an additional therapeutic agent.

17. The method of claim 16, wherein the additional therapeutic agent comprises cisplatin.

18. A composition comprising an ASO that targets a 20-40 nucleotide sequence of lnc-HLX-2-7 (SEQ ID NO:200).

19. The composition of claim 18, wherein the 20-40 nucleotide sequence comprises nucleotides 110-132, nucleotides114-136, nucleotides 169-191, nucleotides 170-192, nucleotides174-196, nucleotides 176-198, nucleotides 183-205, nucleotides 211-233, nucleotides 220-242, nucleotides 222-244, nucleotides 275-297, nucleotides 276-298, nucleotides 321-343, nucleotides 323-345, nucleotides 335-345, nucleotides 331-353, nucleotides 333-355, nucleotides 335-361, nucleotides 350-372, nucleotides 352-374, nucleotides 466-488, nucleotides 468-488, nucleotides 480-500, or nucleotides 494-516.

20. The composition of claim 19, wherein the ASO targeting nucleotides 110-132 comprises SEQ ID NO:269, the ASO targeting nucleotides 114-136 comprises SEQ ID NO:270, wherein the ASO targeting nucleotides 169-191 comprises SEQ ID NO:271, wherein the ASO targeting nucleotides 170-192 comprises SEQ ID NO:272, wherein the ASO targeting nucleotides174-196 comprises SEQ ID NO:273, wherein the ASO targeting nucleotides 176-198 comprises SEQ ID NO:274, wherein the ASO targeting nucleotides 183-205 comprises SEQ ID NO:275, wherein the ASO targeting nucleotides 211-233 comprises SEQ ID NO:276, wherein the ASO targeting nucleotides 220-242 SEQ ID NO:277, wherein the ASO targeting nucleotides 222-244 comprises SEQ ID NO:278, wherein the ASO targeting nucleotides 275-297 comprises SEQ ID NO:279, wherein the ASO targeting nucleotides 276-298 comprises SEQ ID NO:280, wherein the ASO targeting nucleotides 321-343 comprises SEQ ID NO:281, wherein the ASO targeting nucleotides 323-345 comprises SEQ ID NO:282, wherein the ASO targeting nucleotides 331-353 comprises SEQ ID NO:283, wherein the ASO targeting nucleotides 333-355 comprises SEQ ID NO:284, wherein the ASO targeting nucleotides 350-372 comprises SEQ ID NO:285, wherein the ASO targeting nucleotides 352-374 comprises SEQ ID NO:286, wherein the ASO targeting nucleotides 466-488 comprises SEQ ID NO:287, or wherein the ASO targeting nucleotides comprises SEQ ID NO:288.

21. The composition of claim 18, wherein the 20-40 nucleotide sequence comprises nucleotides 325-345, nucleotides 335-361, nucleotides 468-488 or nucleotides 480-500.

22. The composition of claim 21, wherein the ASO targeting nucleotides 325-345 comprises SEQ ID NO:242 or SEQ ID NO:290; wherein the ASO targeting nucleotides 335-361 comprises SEQ ID NO:247 or SEQ ID NO:292; wherein the ASO targeting nucleotides 468-488 comprises SEQ ID NO:240 or SEQ ID NO:289; or wherein the ASO targeting nucleotides 480-500 comprises SEQ ID NO:244 or SEQ ID NO:291.

23. The composition of claim 20, further comprising a polymeric micelle.

24. The composition of claim 23, wherein the polymeric micelle comprises a cerium oxide nanoparticle.


SEQUENCE LISTING (292 sequences)

SEQ ID NO: 1 - RNA, 20 nt, Artificial Sequence
  5' gRNA encompassing the HLX2-7 region
ggacccacucuccaacgcag

SEQ ID NO: 2 - RNA, 20 nt, Artificial Sequence
  3' gRNA encompassing the HLX2-7 region
gcagggaccccucauugacg

SEQ ID NO: 3 - DNA, 21 bp, Artificial Sequence
  lnc_HLX2_71-1R
ccagacataaactcgccaagc

SEQ ID NO: 4 - DNA, 18 bp, Artificial Sequence
  lnc_HLX2_72-1R
ccgcatgtgtccaggcac

SEQ ID NO: 5 - DNA, 26 bp, Artificial Sequence
  lnc_HLX2_73-1R
gtgtgtgtgtgtgtgtgtgtttattc

SEQ ID NO: 6 - DNA, 25 bp, Artificial Sequence
  lnc_HLX2_74-1R
gcagtaacatcgaatgtgtgtgtgt

SEQ ID NO: 7 - DNA, 24 bp, Artificial Sequence
  lnc_HLX2_75-1R
aaaaaaatgaatggatgaaggaat

SEQ ID NO: 8 - DNA, 23 bp, Artificial Sequence
  lnc_HLX2_76-1R
ggatccccataaatactgcaatg

SEQ ID NO: 9 - DNA, 24 bp, Artificial Sequence
  lnc_HLX2_77-1R
tctctatggtgcaaacactcacaa

SEQ ID NO: 10 - DNA, 27 bp, Artificial Sequence
  lnc_HLX2_78-1R
tggttaatgcttagtcaaatacttttt

SEQ ID NO: 11 - DNA, 25 bp, Artificial Sequence
  lnc_HLX2_79-1R
acaaacactgatctcttagctggaa

SEQ ID NO: 12 - DNA, 24 bp, Artificial Sequence
  lnc_HLX2_710-1R
caaccacctcagctattttgaatg

SEQ ID NO: 13 - DNA, 22 bp, Artificial Sequence
  lnc_HLX2_711-1R
gggaatactggctgtcctctcc

SEQ ID NO: 14 - DNA, 26 bp, Artificial Sequence
  lnc_HLX2_712-1R
cctaggaaactaataaacctcttttg

SEQ ID NO: 15 - DNA, 24 bp, Artificial Sequence
  lnc_HLX2_713-1R
gtctgtgtcaatttgtgacagcag

SEQ ID NO: 16 - DNA, 28 bp, Artificial Sequence
  lnc_HLX2_714-1R
acacatttctgttgttttaagtcactaa

SEQ ID NO: 17 - DNA, 26 bp, Artificial Sequence
  lnc_HLX2_715-1R
gagaagcagagaatgtagtgacacaa

SEQ ID NO: 18 - DNA, 22 bp, Artificial Sequence
  lnc_HLX2_716-1R
cagaagaagagtccatgtgcca

SEQ ID NO: 19 - DNA, 23 bp, Artificial Sequence
  lnc_HLX2_717-1R
ggaagacagaggaaaactggatg

SEQ ID NO: 20 - DNA, 22 bp, Artificial Sequence
  lnc_HLX2_718-1R
taccacacgcatgcttatgaga

SEQ ID NO: 21 - DNA, 20 bp, Artificial Sequence
  lnc_HLX2_719-1R
cctgggtggacgctaaatgc

SEQ ID NO: 22 - DNA, 22 bp, Artificial Sequence
  SPRY4-IT11-4R
ggcagatcacttgaggtcagga

SEQ ID NO: 23 - DNA, 20 bp, Artificial Sequence
  SPRY4-IT12-4R
ccttttgggaggccaaggta

SEQ ID NO: 24 - DNA, 22 bp, Artificial Sequence
  SPRY4-IT13-4R
tggctcatgcctgtaatctcag

SEQ ID NO: 25 - DNA, 18 bp, Artificial Sequence
  SPRY4-IT14-4R
aaagaaggcctggcgcag

SEQ ID NO: 26 - DNA, 27 bp, Artificial Sequence
  SPRY4-IT15-4R
aaaaaaaaagaaagaaaaaaagaaaag

SEQ ID NO: 27 - DNA, 24 bp, Artificial Sequence
  SPRY4-IT16-4R
cagcacagctaaatgatgtctcaa

SEQ ID NO: 28 - DNA, 22 bp, Artificial Sequence
  SPRY4-IT17-4R
agctgcctatttaagaacccct

SEQ ID NO: 29 - DNA, 25 bp, Artificial Sequence
  SPRY4-IT18-4R
gctgacaaaggaaaacaattttctg

SEQ ID NO: 30 - DNA, 24 bp, Artificial Sequence
  SPRY4-IT19-4R
aagagcctctgctgaatttatgtg

SEQ ID NO: 31 - DNA, 18 bp, Artificial Sequence
  SPRY4-IT110-4R
caccagcagggaccctcc

SEQ ID NO: 32 - DNA, 18 bp, Artificial Sequence
  SPRY4-IT111-4R
actgctggcctcacccct

SEQ ID NO: 33 - DNA, 23 bp, Artificial Sequence
  SPRY4-IT112-4R
agcaaaaaccaaatcagagttcc

SEQ ID NO: 34 - DNA, 22 bp, Artificial Sequence
  SPRY4-IT113-4R
gattcctttcaaccaccagctc

SEQ ID NO: 35 - DNA, 25 bp, Artificial Sequence
  SPRY4-IT114-4R
ccctattataaccccgatgtagtag

SEQ ID NO: 36 - DNA, 26 bp, Artificial Sequence
  SPRY4-IT115-4R
actgggcatattctaaaatgtatctt

SEQ ID NO: 37 - DNA, 18 bp, Artificial Sequence
  SPRY4-IT116-4R
gcagcatccgatggctcc

SEQ ID NO: 38 - DNA, 19 bp, Artificial Sequence
  SPRY4-IT117-4R
ttggctctctggggacgat

SEQ ID NO: 39 - DNA, 19 bp, Artificial Sequence
  SPRY4-IT118-4R
gagcttggcccacgatgac

SEQ ID NO: 40 - DNA, 18 bp, Artificial Sequence
  SPRY4-IT119-4R
ggccagacatggggatgg

SEQ ID NO: 41 - DNA, 19 bp, Artificial Sequence
  SPRY4-IT120-4R
catctgggcctgcagttga

SEQ ID NO: 42 - DNA, 20 bp, Artificial Sequence
  SPRY4-IT121-4R
cctccagaggcagctgtcaa

SEQ ID NO: 43 - DNA, 21 bp, Artificial Sequence
  SPRY4-IT122-4R
gcattcacaggctcccataac

SEQ ID NO: 44 - DNA, 19 bp, Artificial Sequence
  SPRY4-IT123-4R
gcaggcaatggggatgttg

SEQ ID NO: 45 - DNA, 17 bp, Artificial Sequence
  SPRY4-IT124-4R
ggatgggagcagccgct

SEQ ID NO: 46 - DNA, 19 bp, Artificial Sequence
  SPRY4-IT125-4R
aagtcccaccaggaagcca

SEQ ID NO: 47 - DNA, 21 bp, Artificial Sequence
  SPRY4-IT126-4R
cagattccccaattcatggaa

SEQ ID NO: 48 - DNA, 23 bp, Artificial Sequence
  SPRY4-IT127-4R
taataggccttggaatcagaaag

SEQ ID NO: 49 - DNA, 21 bp, Artificial Sequence
  SPRY4-IT128-4R
atgggcaatgctcagaaattt

SEQ ID NO: 50 - DNA, 26 bp, Artificial Sequence
  SPRY4-IT129-4R
catgtcctacagataaagcaaaagaa

SEQ ID NO: 51 - DNA, 18 bp, Artificial Sequence
  MYC1-6R
cgttgaggggcatcgtcg

SEQ ID NO: 52 - DNA, 24 bp, Artificial Sequence
  MYC2-6R
catagttcctgttggtgaagctaa

SEQ ID NO: 53 - DNA, 21 bp, Artificial Sequence
  MYC3-6R
gcaccgagtcgtagtcgaggt

SEQ ID NO: 54 - DNA, 21 bp, Artificial Sequence
  MYC4-6R
cgtcgcagtagaaatacggct

SEQ ID NO: 55 - DNA, 23 bp, Artificial Sequence
  MYC5-6R
ctgctggtagaagttctcctcct

SEQ ID NO: 56 - DNA, 19 bp, Artificial Sequence
  MYC6-6R
gcagctcgctctgctgctg

SEQ ID NO: 57 - DNA, 13 bp, Artificial Sequence
  MYC7-6R
ggcgccgggggct

SEQ ID NO: 58 - DNA, 22 bp, Artificial Sequence
  MYC8-6R
tttcttccagatatcctcgctg

SEQ ID NO: 59 - DNA, 18 bp, Artificial Sequence
  MYC9-6R
ggtgggcagcagctcgaa

SEQ ID NO: 60 - DNA, 17 bp, Artificial Sequence
  MYC10-6R
ctaggggacaggggcgg

SEQ ID NO: 61 - DNA, 13 bp, Artificial Sequence
  MYC11-6R
cccggagcggcgg

SEQ ID NO: 62 - DNA, 19 bp, Artificial Sequence
  MYC12-6R
cgtaggagggcgagcagag

SEQ ID NO: 63 - DNA, 20 bp, Artificial Sequence
  MYC13-6R
ggagaagggtgtgaccgcaa

SEQ ID NO: 64 - DNA, 19 bp, Artificial Sequence
  MYC14-6R
cgtcgttgtctccccgaag

SEQ ID NO: 65 - DNA, 20 bp, Artificial Sequence
  MYC17-6R
agctcggtcaccatctccag

SEQ ID NO: 66 - DNA, 20 bp, Artificial Sequence
  MYC18-6R
tcaccatgtctcctcccagc

SEQ ID NO: 67 - DNA, 21 bp, Artificial Sequence
  MYC19-6R
ggtcgcagatgaaactctggt

SEQ ID NO: 68 - DNA, 20 bp, Artificial Sequence
  MYC20-6R
gatgaaggtctcgtcgtccg

SEQ ID NO: 69 - DNA, 24 bp, Artificial Sequence
  MYC21-6R
acagtcctggatgatgatgttttt

SEQ ID NO: 70 - DNA, 19 bp, Artificial Sequence
  MYC22-6R
ccgagaagccgctccacat

SEQ ID NO: 71 - DNA, 14 bp, Artificial Sequence
  MYC30-6R
gcggcggcgctcag

SEQ ID NO: 72 - DNA, 20 bp, Artificial Sequence
  MYC31-6R
aggggtcgatgcactctgag

SEQ ID NO: 73 - DNA, 19 bp, Artificial Sequence
  MYC32-6R
gggtaggggaagaccaccg

SEQ ID NO: 74 - DNA, 20 bp, Artificial Sequence
  MYC33-6R
gcgagctgctgtcgttgaga

SEQ ID NO: 75 - DNA, 17 bp, Artificial Sequence
  MYC34-6R
cgaggcgcaggacttgg

SEQ ID NO: 76 - DNA, 20 bp, Artificial Sequence
  MYC35-6R
gagaaggcgctggagtcttg

SEQ ID NO: 77 - DNA, 20 bp, Artificial Sequence
  MYC36-6R
gcagagaatccgaggacgga

SEQ ID NO: 78 - DNA, 20 bp, Artificial Sequence
  MYC37-6R
ggaggactccgtcgaggaga

SEQ ID NO: 79 - DNA, 15 bp, Artificial Sequence
  MYC38-6R
ggggctgccctgcgg

SEQ ID NO: 80 - DNA, 18 bp, Artificial Sequence
  MYC39-6R
atggagcaccaggggctc

SEQ ID NO: 81 - DNA, 18 bp, Artificial Sequence
  MYC40-6R
ggtgggcggtgtctcctc

SEQ ID NO: 82 - DNA, 20 bp, Artificial Sequence
  MYC41-6R
tcctcagagtcgctgctggt

SEQ ID NO: 83 - DNA, 25 bp, Artificial Sequence
  MYC42-6R
gatttcttcctcatcttcttgttcc

SEQ ID NO: 84 - DNA, 24 bp, Artificial Sequence
  MYC43-6R
cctcttttccacagaaacaacatc

SEQ ID NO: 85 - DNA, 19 bp, Artificial Sequence
  MYC44-6R
accttttgccaggagcctg

SEQ ID NO: 86 - DNA, 23 bp, Artificial Sequence
  MYC45-6R
agcagaaggtgatccagactctg

SEQ ID NO: 87 - DNA, 19 bp, Artificial Sequence
  MYC46-6R
gaggtttgctgtggcctcc

SEQ ID NO: 88 - DNA, 20 bp, Artificial Sequence
  MYC47-6R
gaggaccagtgggctgtgag

SEQ ID NO: 89 - DNA, 20 bp, Artificial Sequence
  MYC48-6R
gtggagacgtggcacctctt

SEQ ID NO: 90 - DNA, 21 bp, Artificial Sequence
  MYC49-6R
gctgcgtagttgtgctgatgt

SEQ ID NO: 91 - DNA, 18 bp, Artificial Sequence
  MYC50-6R
ttccgagtggagggaggc

SEQ ID NO: 92 - DNA, 21 bp, Artificial Sequence
  MYC51-6R
ctcttggcagcaggatagtcc

SEQ ID NO: 93 - DNA, 24 bp, Artificial Sequence
  MYC52-6R
actctgacactgtccaacttgacc

SEQ ID NO: 94 - DNA, 23 bp, Artificial Sequence
  MYC53-6R
ggttgttgctgatctgtctcagg

SEQ ID NO: 95 - DNA, 18 bp, Artificial Sequence
  MYC54-6R
tggggctggtgcattttc

SEQ ID NO: 96 - DNA, 18 bp, Artificial Sequence
  MYC55-6R
cctcggtgtccgaggacc

SEQ ID NO: 97 - DNA, 22 bp, Artificial Sequence
  MYC56-6R
tgtgttcgcctcttgacattct

SEQ ID NO: 98 - DNA, 18 bp, Artificial Sequence
  MYC57-6R
tggcgctccaagacgttg

SEQ ID NO: 99 - DNA, 22 bp, Artificial Sequence
  MYC58-6R
ccgttttagctcgttcctcctc

SEQ ID NO: 100 - DNA, 25 bp, Artificial Sequence
  MYC62-6R
tggcttttttaaggataactacctt

SEQ ID NO: 101 - DNA, 21 bp, Artificial Sequence
  MYC63-6R
ggacggacaggatgtatgctg

SEQ ID NO: 102 - DNA, 21 bp, Artificial Sequence
  MYC64-6R
tgagcttttgctcctctgctt

SEQ ID NO: 103 - DNA, 19 bp, Artificial Sequence
  MYCN1-6R
gctcgctggactgagccct

SEQ ID NO: 104 - DNA, 21 bp, Artificial Sequence
  MYCN2-6R
gaaggcatcgtttgaggatca

SEQ ID NO: 105 - DNA, 19 bp, Artificial Sequence
  MYCN3-6R
ttgtgctgctggtggatgg

SEQ ID NO: 106 - DNA, 23 bp, Artificial Sequence
  MYCN6-6R
ctctttatcttcttctgtggggg

SEQ ID NO: 107 - DNA, 16 bp, Artificial Sequence
  MYCN7-6R
acgtggggacgcctcg

SEQ ID NO: 108 - DNA, 20 bp, Artificial Sequence
  MYCN8-6R
gggatgacactcttgagcgg

SEQ ID NO: 109 - DNA, 21 bp, Artificial Sequence
  MYCN9-6R
ctcaagctcttagcctttggg

SEQ ID NO: 110 - DNA, 19 bp, Artificial Sequence
  MYCN10-6R
cgagtcagagtttcggggg

SEQ ID NO: 111 - DNA, 19 bp, Artificial Sequence
  MYCN11-6R
tgcgacgctcactgtcctc

SEQ ID NO: 112 - DNA, 21 bp, Artificial Sequence
  MYCN12-6R
gctccaggatgttgtggtttc

SEQ ID NO: 113 - DNA, 15 bp, Artificial Sequence
  MYCN13-6R
cgttgcggcgctggc

SEQ ID NO: 114 - DNA, 21 bp, Artificial Sequence
  MYCN14-6R
tgagaaagctggaccgaaggt

SEQ ID NO: 115 - DNA, 18 bp, Artificial Sequence
  MYCN15-6R
gcacgtggtccctgagcg

SEQ ID NO: 116 - DNA, 24 bp, Artificial Sequence
  MYCN16-6R
ccttctcattctttaccaactccg

SEQ ID NO: 117 - DNA, 19 bp, Artificial Sequence
  MYCN17-6R
aaaatgaccaccttggcgg

SEQ ID NO: 118 - DNA, 23 bp, Artificial Sequence
  MYCN18-6R
ggacatactcagtggcctttttc

SEQ ID NO: 119 - DNA, 18 bp, Artificial Sequence
  MYCN19-6R
cctcggcctggagggagt

SEQ ID NO: 120 - DNA, 20 bp, Artificial Sequence
  MYCN20-6R
ttccagcaaaagctggtgct

SEQ ID NO: 121 - DNA, 21 bp, Artificial Sequence
  MYCN21-6R
tcttgcctgcaatttttcctt

SEQ ID NO: 122 - DNA, 23 bp, Artificial Sequence
  MYCN22-6R
attttctttagcaactgctgctg

SEQ ID NO: 123 - DNA, 19 bp, Artificial Sequence
  MYCN23-6R
gcaagtccgagcgtgttca

SEQ ID NO: 124 - DNA, 23 bp, Artificial Sequence
  MYCN24-6R
tgtccagttttgagaagcgtcta

SEQ ID NO: 125 - DNA, 22 bp, Artificial Sequence
  MYCN25-6R
aaatgtgcaaagtggcagtgac

SEQ ID NO: 126 - DNA, 27 bp, Artificial Sequence
  MYCN26-6R
cacaatgtttgtttaaaaaaaaaatca

SEQ ID NO: 127 - DNA, 27 bp, Artificial Sequence
  MYCN27-6R
aaagtaaaccaacattcttaatgtcaa

SEQ ID NO: 128 - DNA, 19 bp, Artificial Sequence
  MYCN28-6R
tcgacaggggaccgatttg

SEQ ID NO: 129 - DNA, 18 bp, Artificial Sequence
  MYCN29-6R
gcccacccagagccgaac

SEQ ID NO: 130 - DNA, 21 bp, Artificial Sequence
  MYCN30-6R
ccccacactggtggtcctact

SEQ ID NO: 131 - DNA, 20 bp, Artificial Sequence
  MYCN31-6R
tctccaaggtcccagcagaa

SEQ ID NO: 132 - DNA, 21 bp, Artificial Sequence
  MYCN34-6R
catggaggtgaggtggaggag

SEQ ID NO: 133 - DNA, 20 bp, Artificial Sequence
  MYCN35-6R
tcaccaacgtttagcgctgt

SEQ ID NO: 134 - DNA, 18 bp, Artificial Sequence
  MYCN36-6R
cccagaggctcccaaccg

SEQ ID NO: 135 - DNA, 23 bp, Artificial Sequence
  MYCN37-6R
acacacaaggtgacttcaacagc

SEQ ID NO: 136 - DNA, 23 bp, Artificial Sequence
  MYCN38-6R
tttctgttgtttggaaacttgga

SEQ ID NO: 137 - DNA, 25 bp, Artificial Sequence
  MYCN39-6R
caccattttaaaaagaaggaatgac

SEQ ID NO: 138 - DNA, 22 bp, Artificial Sequence
  MYCN40-6R
gtggcatctgctggaacttaag

SEQ ID NO: 139 - DNA, 22 bp, Artificial Sequence
  MYCN41-6R
tatcaaatggcaaaccccttat

SEQ ID NO: 140 - DNA, 19 bp, Artificial Sequence
  MYCN42-6R
cagaaatgttccccagggg

SEQ ID NO: 141 - DNA, 23 bp, Artificial Sequence
  MYCN43-6R
ggcggatgtgtcaatggtattta

SEQ ID NO: 142 - DNA, 26 bp, Artificial Sequence
  MYCN44-6R
tctcattacccaggatgtatacaaaa

SEQ ID NO: 143 - DNA, 16 bp, Artificial Sequence
  MYCN45-6R
ggccgcaaaagccacc

SEQ ID NO: 144 - DNA, 29 bp, Artificial Sequence
  MYCN46-6R
acttaggtatgaacttccagtctaatact

SEQ ID NO: 145 - DNA, 27 bp, Artificial Sequence
  MYCN47-6R
cctcaaacattgaggtattattacagt

SEQ ID NO: 146 - DNA, 35 bp, Artificial Sequence
  MYCN54-6R
catatatatatagtaaatttctttacaaaagtttc

SEQ ID NO: 147 - DNA, 23 bp, Artificial Sequence
  MYCN55-6R
gaagaaacaggctaggaaaaagg

SEQ ID NO: 148 - DNA, 26 bp, Artificial Sequence
  MYCN56-6R
ccaaacatgaacaaatacattaacag

SEQ ID NO: 149 - DNA, 23 bp, Artificial Sequence
  MYCN57-6R
ttgcatttacccagttctatgca

SEQ ID NO: 150 - DNA, 27 bp, Artificial Sequence
  MYCN58-6R
cattttgaagaaattaaacacagaact

SEQ ID NO: 151 - DNA, 28 bp, Artificial Sequence
  MYCN59-6R
tgctataagatgcagcactaaatatata

SEQ ID NO: 152 - DNA, 27 bp, Artificial Sequence
  MYCN60-6R
ttttcataaacatgaggtatttcaaag

SEQ ID NO: 153 - DNA, 23 bp, Artificial Sequence
  MALAT11-4R
caggctggttatgactcagaaga

SEQ ID NO: 154 - DNA, 22 bp, Artificial Sequence
  MALAT12-4R
tgcatctaggccatcatactgc

SEQ ID NO: 155 - DNA, 23 bp, Artificial Sequence
  MALAT13-4R
attcaccaaggagctgttttctc

SEQ ID NO: 156 - DNA, 28 bp, Artificial Sequence
  MALAT14-4R
atataatcttttctgcctttacttatca

SEQ ID NO: 157 - DNA, 23 bp, Artificial Sequence
  MALAT15-4R
ttattccccaatggaggtatgac

SEQ ID NO: 158 - DNA, 26 bp, Artificial Sequence
  MALAT16-4R
cagtagtaagaatctcagggttatgc

SEQ ID NO: 159 - DNA, 25 bp, Artificial Sequence
  MALAT17-4R
tggcatatgcagataatgttctcat

SEQ ID NO: 160 - DNA, 25 bp, Artificial Sequence
  MALAT18-4R
tagctttcatttgcttaaaattttt

SEQ ID NO: 161 - DNA, 25 bp, Artificial Sequence
  MALAT19-4R
ggtagattccgtaactttaaattgg

SEQ ID NO: 162 - DNA, 26 bp, Artificial Sequence
  MALAT110-4R
gcttgacaagcaattaactttaaaat

SEQ ID NO: 163 - DNA, 27 bp, Artificial Sequence
  MALAT111-4R
catcaattcattatttttgtggttata

SEQ ID NO: 164 - DNA, 25 bp, Artificial Sequence
  MALAT112-4R
gacattgcctcttcattgtatttct

SEQ ID NO: 165 - DNA, 26 bp, Artificial Sequence
  MALAT113-4R
ttttgtaaaagcagtattttgagatg

SEQ ID NO: 166 - DNA, 24 bp, Artificial Sequence
  MALAT114-4R
catttcttttcgcttttattctgc

SEQ ID NO: 167 - DNA, 27 bp, Artificial Sequence
  MALAT115-4R
tccaggattaatgtagtgtaacatttt

SEQ ID NO: 168 - DNA, 25 bp, Artificial Sequence
  MALAT116-4R
tctcatttatttcggcttcttttat

SEQ ID NO: 169 - DNA, 23 bp, Artificial Sequence
  MALAT117-4R
aatccacttgatcccaactcatc

SEQ ID NO: 170 - DNA, 20 bp, Artificial Sequence
  MALAT118-4R
gcacacagcacagcctcctc

SEQ ID NO: 171 - DNA, 22 bp, Artificial Sequence
  MALAT119-4R
gtctgaggcaaacgaaacattg

SEQ ID NO: 172 - DNA, 27 bp, Artificial Sequence
  MALAT120-4R
aactcttctgataacgaagagatacct

SEQ ID NO: 173 - DNA, 21 bp, Artificial Sequence
  MALAT121-4R
tgctcccagatgaaatgaagc

SEQ ID NO: 174 - DNA, 22 bp, Artificial Sequence
  MALAT122-4R
ttaacagctgcctgctgttttc

SEQ ID NO: 175 - DNA, 25 bp, Artificial Sequence
  MALAT123-4R
tgcagatgcaagttaaacttatctg

SEQ ID NO: 176 - DNA, 25 bp, Artificial Sequence
  MALAT124-4R
agcacttatccctaacatgcaatac

SEQ ID NO: 177 - DNA, 27 bp, Artificial Sequence
  MALAT125-4R
ttaagaactccacagctcttaaaaata

SEQ ID NO: 178 - DNA, 23 bp, Artificial Sequence
  MALAT126-4R
ggagaaagtgccatggttgatat

SEQ ID NO: 179 - DNA, 19 bp, Artificial Sequence
  MALAT127-4R
tcccctagggaaggggtca

SEQ ID NO: 180 - DNA, 24 bp, Artificial Sequence
  MALAT128-4R
tggaaaaatttctcaatcctgaaa

SEQ ID NO: 181 - DNA, 23 bp, Artificial Sequence
  MALAT129-4R
cctacaattttaaaaaggctcga

SEQ ID NO: 182 - DNA, 21 bp, Artificial Sequence
  MALAT130-4R
ctgaagcccacaggaacaagt

SEQ ID NO: 183 - DNA, 26 bp, Artificial Sequence
  MALAT131-4R
tctgagtgaagtgtactatcccatca

SEQ ID NO: 184 - DNA, 25 bp, Artificial Sequence
  MALAT132-4R
gaaattatttaaagatgcaaatgcc

SEQ ID NO: 185 - DNA, 26 bp, Artificial Sequence
  MALAT133-4R
gcactgatcactttagaggcttttaa

SEQ ID NO: 186 - DNA, 24 bp, Artificial Sequence
  MALAT134-4R
caaatttccttagttggcatcaag

SEQ ID NO: 187 - DNA, 23 bp, Artificial Sequence
  MALAT135-4R
gccttcagagattcaatgctaaa

SEQ ID NO: 188 - DNA, 25 bp, Artificial Sequence
  MALAT136-4R
cacatcatgctattcctttcataga

SEQ ID NO: 189 - DNA, 28 bp, Artificial Sequence
  MALAT137-4R
ttttagcagtaacatctgattctaacag

SEQ ID NO: 190 - DNA, 28 bp, Artificial Sequence
  MALAT138-4R
ctacacaatttacatcacaacatgtaaa

SEQ ID NO: 191 - DNA, 28 bp, Artificial Sequence
  MALAT139-4R
ttattattttgaatgatttaatggtttt

SEQ ID NO: 192 - DNA, 33 bp, Artificial Sequence
  MALAT140-4R
ttctaaaagtatacattctctaataaaaatagt

SEQ ID NO: 193 - DNA, 29 bp, Artificial Sequence
  MALAT141-4R
cactattttatttaaataaggagacagct

SEQ ID NO: 194 - DNA, 24 bp, Artificial Sequence
  MALAT142-4R
ccccaacactgaactacagacaaa

SEQ ID NO: 195 - DNA, 20 bp, Artificial Sequence
  MALAT143-4R
aagaatcccccccaagattg

SEQ ID NO: 196 - DNA, 27 bp, Artificial Sequence
  MALAT144-4R
gcagacaaagtttctgaaagattagag

SEQ ID NO: 197 - DNA, 25 bp, Artificial Sequence
  MALAT145-4R
tgatctggtccattaaagagtgttc

SEQ ID NO: 198 - DNA, 20 bp, Artificial Sequence
  MALAT146-4R
tcgttcttccgctcaaatcc

SEQ ID NO: 199 - DNA, 25 bp, Artificial Sequence
  MALAT147-4R
tgtctttcctgccttaaagttacat

SEQ ID NO: 200 - RNA, 517 nt, Homo sapiens
  misc_feature (1)..(517): HLX2-7 lnc RNA sequence
augaccucaaacacuugugcuuggcgaguuuaugucugggugccuggacacaugcgggaauaaacacacacacacacacacacacacacacacauucgauguuacugcauuccuucauccauucauuuuuuucauugcaguauuuauggggauccuugugaguguuugcaccauagagaaaaaaguauuugacuaagcauuaaccauuccagcuaagagaucaguguuugucauucaaaauagcugaggugguugggagaggacagccaguauuccccaaaagagguuuauuaguuuccuaggcugcugucacaaauugacacagacuuagugacuuaaaacaacagaaauguguuugugucacuacauucucugcuucucuggcacauggacucuucuucugcauccaguuuuccucugucuuccucucauaagcaugcgugugguagcauuuagcguccacccaggcaaucuagauuaaucucucaccucaagcuccuuaacugaaucacaucug

SEQ ID NO: 201 - RNA, 703 nt, Homo sapiens
  misc_feature (1)..(703): SPRY4-IT lncRNA sequence
guagagauggggguuucauccuguuggucaggcuggucuugaacuccugaccucaagugaucugccuaccuuggccucccaaaaggcugagauuacaggcaugagccacugcgccaggccuucuuucuuuucuuuuuuucuuucuuuuuuuuuuugagacaucauuuagcugugcugagggguucuuaaauaggcagcucagaaaauuguuuuccuuugucagccacauaaauucagcagaggcucuuggagggucccugcuggugaggggugaggccagcaguggaacucugauuugguuuuugcugagcuggugguugaaaggaauccuacuacaucgggguuauaauagggaagauacauuuuagaauaugcccaguggagccaucggaugcugcaucguccccagagagccaagucaucgugggccaagcucccauccccaugucuggccucaacugcaggcccagauguugacagcugccucuggaggguuaugggagccugugaaugccaacauccccauugccugcagcggcugcucccauccuggcuuccuggugggacuuuuccaugaauuggggaaucugcuuucugauuccaaggccuauuaaaauuucugagcauugcccauuucuuuugcuuuaucuguaggacaugggcuguuuuuaaagaaccucacaaaugaaaaaaaaaaaaaaaa

SEQ ID NO: 202 - RNA, 3721 nt, Homo sapiens
  misc_feature (1)..(3721): MYC RNA sequence
aacucgcuguaguaauuccagcgagaggcagagggagcgagcgggcggccggcuaggguggaagagccgggcgagcagagcugcgcugcgggcguccugggaagggagauccggagcgaauagggggcuucgccucuggcccagcccucccgcugaucccccagccagcgguccgcaacccuugccgcauccacgaaacuuugcccauagcagcgggcgggcacuuugcacuggaacuuacaacacccgagcaaggacgcgacucucccgacgcggggaggcuauucugcccauuuggggacacuuccccgccgcugccaggacccgcuucucugaaaggcucuccuugcagcugcuuagacgcuggauuuuuuucggguaguggaaaaccagcagccucccgcgacgaugccccucaacguuagcuucaccaacaggaacuaugaccucgacuacgacucggugcagccguauuucuacugcgacgaggaggagaacuucuaccagcagcagcagcagagcgagcugcagcccccggcgcccagcgaggauaucuggaagaaauucgagcugcugcccaccccgccccuguccccuagccgccgcuccgggcucugcucgcccuccuacguugcggucacacccuucucccuucggggagacaacgacggcgguggcgggagcuucuccacggccgaccagcuggagauggugaccgagcugcugggaggagacauggugaaccagaguuucaucugcgacccggacgacgagaccuucaucaaaaacaucaucauccaggacuguauguggagcggcuucucggccgccgccaagcucgucucagagaagcuggccuccuaccaggcugcgcgcaaagacagcggcagcccgaaccccgcccgcggccacagcgucugcuccaccuccagcuuguaccugcaggaucugagcgccgccgccucagagugcaucgaccccucgguggucuuccccuacccucucaacgacagcagcucgcccaaguccugcgccucgcaagacuccagcgccuucucuccguccucggauucucugcucuccucgacggaguccuccccgcagggcagccccgagccccuggugcuccaugaggagacaccgcccaccaccagcagcgacucugaggaggaacaagaagaugaggaagaaaucgauguuguuucuguggaaaagaggcaggcuccuggcaaaaggucagagucuggaucaccuucugcuggaggccacagcaaaccuccucacagcccacugguccucaagaggugccacgucuccacacaucagcacaacuacgcagcgccucccuccacucggaaggacuauccugcugccaagagggucaaguuggacagugucagaguccugagacagaucagcaacaaccgaaaaugcaccagccccagguccucggacaccgaggagaaugucaagaggcgaacacacaacgucuuggagcgccagaggaggaacgagcuaaaacggagcuuuuuugcccugcgugaccagaucccggaguuggaaaacaaugaaaaggcccccaagguaguuauccuuaaaaaagccacagcauacauccuguccguccaagcagaggagcaaaagcucauuucugaagaggacuuguugcggaaacgacgagaacaguugaaacacaaacuugaacagcuacggaacucuugugcguaaggaaaaguaaggaaaacgauuccuucuaacagaaauguccugagcaaucaccuaugaacuuguuucaaaugcaugaucaaaugcaaccucacaaccuuggcugagucuugagacugaaagauuuagccauaauguaaacugccucaaauuggacuuugggcauaaaagaacuuuuuuaugcuuaccaucuuuuuuuuuucuuuaacagauuuguauuuaagaauuguuuuuaaaaaauuuuaagauuuacacaauguuucucuguaaauauugccauuaaauguaaauaacuuuaauaaaacguuuauagcaguuacacagaauuucaauccuaguauauaguaccuaguauuauagguacuauaaacccuaauuuuuuuuauuuaaguacauuuugcuuuuuaaaguugauuuuuuucuauuguuuuuagaaaaaauaaaauaacuggcaaauauaucauugagccaaaucuuaaguugugaauguuuuguuucguuucuucccccucccaaccaccaccaucccuguuuguuuucaucaauugccccuucagaggguggucuuaagaaaggcaagaguuuuccucuguugaaaugggucugggggccuuaaggucuuuaaguucuuggagguucuaagaugcuuccuggagacuaugauaacagccagaguugacaguuagaaggaauggcagaaggcaggugagaaggugagagguaggcaaaggagauacaagaggucaaagguagcaguuaaguacacaaagaggcauaaggacuggggaguugggaggaaggugaggaagaaacuccuguuacuuuaguuaaccagugccaguccccugcucacuccaaacccaggaauucugcccaguugauggggacacggugggaaccagcuucugcugccuucacaaccaggcgccaguccuguccauggguuaucucgcaaaccccagaggaucucugggaggaaugcuacuauuaacccuauuucacaaacaaggaaauagaagagcucaaagagguuauguaacuuaucuguagccacgcagauaauacaaagcagcaaucuggacccauucuguucaaaacacuuaacccuucgcuaucaugccuugguucaucugggucuaaugugcugagaucaagaagguuuaggaccuaauggacagacucaagucauaacaaugcuaagcucuauuugugucccaagcacuccuaagcauuuuaucccuaacucuacaucaaccccaugaaggagauacuguugauuuccccauauuagaaguagagagggaagcugaggcacacaaagacucauccacaugcccaagauucacugauagggaaaaguggaagcgagauuugaacccaggcuguuuacuccuaaccuguccaagccaccucucagacgacgguaggaaucagcuggcugcuugugaguacaggaguuacaguccaguggguuauguuuuuuaagucucaacaucuaagccuggucaggcaucaguuccccuuuuuuugugauuuauuuuguuuuuauuuuguuguucauuguuuaauuuuuccuuuuacaaugagaaggucaccaucuugacuccuaccuuagccauuuguugaaucagacucaugacggcuccugggaagaagccaguucagaucauaaaauaaaacauauuuauucuuugucaugggagucauuauuuuagaaacuacaaacucuccuugcuuccauccuuuuuuacauacucaugacacaugcucauccugaguccuugaaaagguauuuuugaacauguguauuaauuauaagccucugaaaaccuauggcccaaaccagaaaugauguugauuauauagguaaaugaaggaugcuauugcuguucuaauuaccucauugucucagucucaaaguaggucuucagcucccuguacuuugggauuuuaaucuaccaccacccauaaaucaauaaauaauuacuuucuuuga

SEQ ID NO: 203 - RNA, 2923 nt, Homo sapiens
  misc_feature (1)..(2923): MYCN RNA sequence
aggcugugacagucaucugucuggacgcgcuggguggaugcggggggcuccugggaacuguguuggagccgagcaagcgcuagccaggcgcaagcgcgcacagacuguagccauccgaggacacccccgcccccccggcccacccggagacacccgcgcagaaucgccuccggauccccugcagucggcgggagguaaggagcagggcuugcaaaccgcccggcgcccagggaagcgacgagcgccggggcaaggcaagcccuggacgggauugcgacgugcgcaccgggcgcccuaauaugcccgggggacuguuucugcuuccgaaacaaaaccaucucuggguuuucccagaaaagccaguuccagccccgaaggcauccuggcuagaggagacccgcccuaauccuuuugcagcccuuaccggggggaguaauggcuucugcgaaaagaaauucccucggcucuagaagaucugucuguguuugagcugucggagagccguguuggaggucggcgccggcccccgccuuccgcgccccccacgggaaggaagcacccccgguauuaaaacgaacggggcggaaagaagcccucagucgccggccgggaggcgagccgaugccgagcugcuccacguccaccaugccgggcaugaucugcaagaacccagaccucgaguuugacucgcuacagcccugcuucuacccggacgaagaugacuucuacuucggcggccccgacucgacccccccgggggaggacaucuggaagaaguuugagcugcugcccacgcccccgcugucgcccagccguggcuucgcggagcacagcuccgagcccccgagcugggucacggagaugcugcuugagaacgagcuguggggcagcccggccgaggaggacgcguucggccuggggggacuggguggccucacccccaacccggucauccuccaggacugcauguggagcggcuucuccgcccgcgagaagcuggagcgcgccgugagcgagaagcugcagcacggccgcgggccgccaaccgccgguuccaccgcccaguccccgggagccggcgccgccagcccugcgggucgcgggcacggcggggcugcgggagccggccgcgccggggccgcccugcccgccgagcucgcccacccggccgccgagugcguggaucccgccguggucuuccccuuucccgugaacaagcgcgagccagcgcccgugcccgcagccccggccagugccccggcggcgggcccugcggucgccucgggggcggguauugccgccccagccggggccccgggggucgccccuccgcgcccaggcggccgccagaccagcggcggcgaccacaaggcccucaguaccuccggagaggacacccugagcgauucagaugaugaagaugaugaagaggaagaugaagaggaagaaaucgacguggucacuguggagaagcggcguuccuccuccaacaccaaggcugucaccacauucaccaucacugugcgucccaagaacgcagcccugggucccgggagggcucaguccagcgagcugauccucaaacgaugccuucccauccaccagcagcacaacuaugccgcccccucucccuacguggagagugaggaugcacccccacagaagaagauaaagagcgaggcguccccacguccgcucaagagugucauccccccaaaggcuaagagcuugagcccccgaaacucugacucggaggacagugagcgucgcagaaaccacaacauccuggagcgccagcgccgcaacgaccuucgguccagcuuucucacgcucagggaccacgugccggaguugguaaagaaugagaaggccgccaagguggucauuuugaaaaaggccacugaguauguccacucccuccaggccgaggagcaccagcuuuugcuggaaaaggaaaaauugcaggcaagacagcagcaguugcuaaagaaaauugaacacgcucggacuugcuagacgcuucucaaaacuggacagucacugccacuuugcacauuuugauuuuuuuuuuaaacaaacauuguguugacauuaagaauguugguuuacuuucaaaucgguccccugucgaguucggcucugggugggcaguaggaccaccagugugggguucugcugggaccuuggagagccugcaucccaggaugcuggguggcccugcagccuccuccaccucaccuccaugacagcgcuaaacguuggugacgguugggagccucuggggcuguugaagucaccuuguguguuccaaguuuccaaacaacagaaagucauuccuucuuuuuaaaauggugcuuaaguuccagcagaugccacauaagggguuugccauuugauaccccuggggaacauuucuguaaauaccauugacacauccgccuuuuguauacauccuggguaaugagagguggcuuuugcggccaguauuagacuggaaguucauaccuaaguacuguaauaauaccucaauguuugaggagcauguuuuguauacaaauauauuguuaaucucuguuauguacuguacuaauucuuacacugccuguauacuuuaguaugacgcugauacauaacuaaauuugauacuuauauuuucguaugaaaaugaguugugaaaguuuugaguagauauuacuuuaucacuuuuugaacuaagaaacuuuuguaaagaaauuuacuauauauauaugccuuuuuccuagccuguuucuuccuguuaauguauuuguucauguuuggugcauagaacuggguaaaugcaaaguucuguguuuaauuucuucaaaauguauauauuuagugcugcaucuuauagcacuuugaaauaccucauguuuaugaaaauaaauagcuuaaaauuaaa

SEQ ID NO: 204 - DNA, 21 bp, Artificial Sequence
  ACTB forward primer
cctggcattgccgacaggatg

SEQ ID NO: 205 - DNA, 23 bp, Artificial Sequence
  ACTB reverse primer
ccgatccacacggagtacttgcg

SEQ ID NO: 206 - DNA, 20 bp, Artificial Sequence
  lnc-HLX-2-7 forward primer
gcttctctggcacatggact

SEQ ID NO: 207 - DNA, 20 bp, Artificial Sequence
  lnc-HLX-2-7 reverse primer
gtccttcgtgagcacagcat

SEQ ID NO: 208 - DNA, 20 bp, Artificial Sequence
  HLX forward primer
gcttctctggcacatggact

SEQ ID NO: 209 - DNA, 20 bp, Artificial Sequence
  HLX reverse primer
gtccttcgtgagcacagcat

SEQ ID NO: 210 - DNA, 20 bp, Artificial Sequence
  MYC forward primer
aaaggcccccaaggtagtta

SEQ ID NO: 211 - DNA, 20 bp, Artificial Sequence
  MYC reverse primer
gcacaagagttccgtagctg

SEQ ID NO: 212 - DNA, 20 bp, Artificial Sequence
  MYCN forward primer
ctaatactggccgcaaaagc

SEQ ID NO: 213 - DNA, 20 bp, Artificial Sequence
  MYCN reverse primer
cataaggggtttgccatttg

SEQ ID NO: 214 - DNA, 24 bp, Artificial Sequence
  PTGR1 forward primer
cagacacaataccactgtctttgg

SEQ ID NO: 215 - DNA, 24 bp, Artificial Sequence
  PTGR1 reverse primer
ctgcattaaccatcactgtttctc

SEQ ID NO: 216 - DNA, 21 bp, Artificial Sequence
  FZD6 forward primer
agactctctggggaacaggtc

SEQ ID NO: 217 - DNA, 25 bp, Artificial Sequence
  FZD6 reverse primer
ggccagtgtcagtaatatcactctt

SEQ ID NO: 218 - DNA, 25 bp, Artificial Sequence
  TRPM3 forward primer
aatacttcagagaaaaggatgatcg

SEQ ID NO: 219 - DNA, 23 bp, Artificial Sequence
  TRPM3 reverse primer
gagtgctctctctcgttgacttc

SEQ ID NO: 220 - DNA, 25 bp, Artificial Sequence
  NAMPT forward primer
aaaagggccgattatctttacatag

SEQ ID NO: 221 - DNA, 25 bp, Artificial Sequence
  NAMPT reverse primer
ccattcttgaagacagtatggagaa

SEQ ID NO: 222 - DNA, 19 bp, Artificial Sequence
  NRBP2 forward primer
aggacgagagcgacatcct

SEQ ID NO: 223 - DNA, 21 bp, Artificial Sequence
  NRBP2 reverse primer
ggctaggaaggtgctctgaag

SEQ ID NO: 224 - DNA, 25 bp, Artificial Sequence
  NBAT1 forward primer
gtttatccatcttcagctccactct

SEQ ID NO: 225 - DNA, 23 bp, Artificial Sequence
  NBAT1 reverse primer
tctgtgggtttcagtttcttcat

SEQ ID NO: 226 - DNA, 26 bp, Artificial Sequence
  CCNG2 forward primer
caacagctactatagtgttcctgagc

SEQ ID NO: 227 - DNA, 25 bp, Artificial Sequence
  CCNG2 reverse primer
tctcctctccacaactcatatcttc

SEQ ID NO: 228 - DNA, 25 bp, Artificial Sequence
  ELK4 forward primer
gcaagaacaagcctaacatgaatta

SEQ ID NO: 229 - DNA, 25 bp, Artificial Sequence
  ELK4 reverse primer
acacaaacttctgaccattcacttt

SEQ ID NO: 230 - DNA, 25 bp, Artificial Sequence
  CDKN2C forward primer
ttgcaaaataatgtaaacgtcaatg

SEQ ID NO: 231 - DNA, 25 bp, Artificial Sequence
  CDKN2C reverse primer
ttagcacctctaagtagcagtctcc

SEQ ID NO: 232 - DNA, 25 bp, Artificial Sequence
  CDK6 forward primer
caaccaattgagaagtttgtaacag

SEQ ID NO: 233 - DNA, 24 bp, Artificial Sequence
  CDK6 reverse primer
ggcactgtaggcagatattctttt

SEQ ID NO: 234 - DNA, 25 bp, Artificial Sequence
  HLX-2KB forward primer
ttatttcttaagagagagggtgagg

SEQ ID NO: 235 - DNA, 25 bp, Artificial Sequence
  HLX-2KB reverse primer
aatttgactgcaaacatttagacct

SEQ ID NO: 236 - DNA, 23 bp, Artificial Sequence
  HLX-TSS forward primer
tacgcagagtagcaagaagcact

SEQ ID NO: 237 - DNA, 21 bp, Artificial Sequence
  HLX-TSS reverse primer
tggaggggaattaggaacaag

SEQ ID NO: 238 - DNA, 25 bp, Artificial Sequence
  E-box forward primer
taataaacaaaaccgcctagatgag

SEQ ID NO: 239 - DNA, 25 bp, Artificial Sequence
  E-box reverse primer
aaaggctttacataaatcggcttac

SEQ ID NO: 240 - DNA, 21 bp, Artificial Sequence
  Antisense oligonucleotide targeting lnc-HLX-2-7
  misc_feature (1)..(20): Phosphorothioate linkages
  misc_feature (1)..(3): Locked nucleic acid (LNA)
  misc_feature (19)..(21): Locked nucleic acid (LNA)
tgagagattaatctagattgc

SEQ ID NO: 241 - DNA, 21 bp, Artificial Sequence
  control antisense olignucleotide targeting luciferase
  misc_feature (1)..(20): Phosphorotioate linkages
  misc_feature (1)..(3): Locked nucleic acid (LNA)
  misc_feature (19)..(21): Locked nucleic acid (LNA)
tcgaagtactcagcgtaagtt

SEQ ID NO: 242 - DNA, 21 bp, Artificial Sequence
  Antisense oligonucleotide targeting lnc-HLX-2-7
  misc_feature (1)..(20): Phosphorothioate linkages
  misc_feature (1)..(3): Locked nucleic acid (LNA)
  misc_feature (19)..(21): Phosphorothioate linkages
gttgttttaagtcactaagtc

SEQ ID NO: 243 - DNA, 21 bp, Homo sapiens
gacttagtgacttaaaacaac

SEQ ID NO: 244 - DNA, 21 bp, Artificial Sequence
  Antisense oligonucleotide target lnc-HLX2-7
  misc_feature (1)..(20): Phosphorothioate linkages
  misc_feature (1)..(3): Locked nucleic acid (LNA)
  misc_feature (19)..(21): Locked nucleic acid (LNA)
aggagcttgaggtgagagatt

SEQ ID NO: 245 - DNA, 21 bp, Homo sapiens
aatctctcacctcaagctcct

SEQ ID NO: 246 - DNA, 21 bp, Homo sapiens
gcaatctagattaatctctca

SEQ ID NO: 247 - DNA, 21 bp, Artificial Sequence
  Antisense oligonucleotide targeting lnc-HLX2-7
  misc_feature (1)..(20): Phosphorothioate linkages
  misc_feature (1)..(3): Locked nucleic acid (LNA)
  misc_feature (19)..(21): Locked nucleic acid (LNA)
acacatttctgttgttttaag

SEQ ID NO: 248 - DNA, 21 bp, Homo sapiens
cttaaaacaacagaaatgtgt

SEQ ID NO: 249 - DNA, 23 bp, Homo sapiens
ttccttcatccattcattttttt

SEQ ID NO: 250 - DNA, 23 bp, Homo sapiens
ttcatccattcatttttttcatt

SEQ ID NO: 251 - DNA, 23 bp, Homo sapiens
caccatagagaaaaaagtatttg

SEQ ID NO: 252 - DNA, 23 bp, Homo sapiens
accatagagaaaaaagtatttga

SEQ ID NO: 253 - DNA, 23 bp, Homo sapiens
tagagaaaaaagtatttgactaa

SEQ ID NO: 254 - DNA, 23 bp, Homo sapiens
gagaaaaaagtatttgactaagc

SEQ ID NO: 255 - DNA, 23 bp, Homo sapiens
aagtatttgactaagcattaacc

SEQ ID NO: 256 - DNA, 23 bp, Homo sapiens
agctaagagatcagtgtttgtca

SEQ ID NO: 257 - DNA, 23 bp, Homo sapiens
atcagtgtttgtcattcaaaata

SEQ ID NO: 258 - DNA, 23 bp, Homo sapiens
cagtgtttgtcattcaaaatagc

SEQ ID NO: 259 - DNA, 23 bp, Homo sapiens
ccccaaaagaggtttattagttt

SEQ ID NO: 260 - DNA, 23 bp, Homo sapiens
cccaaaagaggtttattagtttc

SEQ ID NO: 261 - DNA, 23 bp, Homo sapiens
cacagacttagtgacttaaaaca

SEQ ID NO: 262 - DNA, 23 bp, Homo sapiens
cagacttagtgacttaaaacaac

SEQ ID NO: 263 - DNA, 23 bp, Homo sapiens
gtgacttaaaacaacagaaatgt

SEQ ID NO: 264 - DNA, 23 bp, Homo sapiens
gacttaaaacaacagaaatgtgt

SEQ ID NO: 265 - DNA, 23 bp, Homo sapiens
atgtgtttgtgtcactacattct

SEQ ID NO: 266 - DNA, 23 bp, Homo sapiens
gtgtttgtgtcactacattctct

SEQ ID NO: 267 - DNA, 23 bp, Homo sapiens
aggcaatctagattaatctctca

SEQ ID NO: 268 - DNA, 23 bp, Homo sapiens
agctccttaactgaatcacatct

SEQ ID NO: 269 - DNA, 23 bp, Artificial Sequence
  ASO that targets SEQ ID NO:249
aaaaaaatgaatggatgaaggaa

SEQ ID NO: 270 - DNA, 23 bp, Artificial Sequence
  ASO that targets SEQ ID NO:250
aatgaaaaaaatgaatggatgaa

SEQ ID NO: 271 - DNA, 23 bp, Artificial Sequence
  ASO that targets SEQ ID NO:251
caaatacttttttctctatggtg

SEQ ID NO: 272 - DNA, 23 bp, Artificial Sequence
  ASO that targets SEQ ID NO:252
tcaaatacttttttctctatggt

SEQ ID NO: 273 - DNA, 23 bp, Artificial Sequence
  ASO that targets SEQ ID NO:253
ttagtcaaatacttttttctcta

SEQ ID NO: 274 - DNA, 23 bp, Artificial Sequence
  ASO that targets SEQ ID NO:254
gcttagtcaaatacttttttctc

SEQ ID NO: 275 - DNA, 23 bp, Artificial Sequence
  ASO that targets SEQ ID NO:255
ggttaatgcttagtcaaatactt

SEQ ID NO: 276 - DNA, 23 bp, Artificial Sequence
  ASO that targets SEQ ID NO:256
tgacaaacactgatctcttagct

SEQ ID NO: 277 - DNA, 23 bp, Artificial Sequence
  ASO that targets SEQ ID NO:257
tattttgaatgacaaacactgat

SEQ ID NO: 278 - DNA, 23 bp, Artificial Sequence
  ASO that targets SEQ ID NO:258
gctattttgaatgacaaacactg

SEQ ID NO: 279 - DNA, 23 bp, Artificial Sequence
  ASO that targets SEQ ID NO:259
aaactaataaacctcttttgggg

SEQ ID NO: 280 - DNA, 23 bp, Artificial Sequence
  ASO that targets SEQ ID NO:260
gaaactaataaacctcttttggg

SEQ ID NO: 281 - DNA, 23 bp, Artificial Sequence
  ASO that targets SEQ ID NO:261
tgttttaagtcactaagtctgtg

SEQ ID NO: 282 - DNA, 23 bp, Artificial Sequence
  ASO that targets SEQ ID NO:262
gttgttttaagtcactaagtctg

SEQ ID NO: 283 - DNA, 23 bp, Artificial Sequence
  ASO that targets SEQ ID NO:263
acatttctgttgttttaagtcac

SEQ ID NO: 284 - DNA, 23 bp, Artificial Sequence
  ASO that targets SEQ ID NO:264
acacatttctgttgttttaagtc

SEQ ID NO: 285 - DNA, 23 bp, Artificial Sequence
  ASO that targets SEQ ID NO:265
agaatgtagtgacacaaacacat

SEQ ID NO: 286 - DNA, 23 bp, Artificial Sequence
  ASO that targets SEQ ID NO:266
agagaatgtagtgacacaaacac

SEQ ID NO: 287 - DNA, 23 bp, Artificial Sequence
  ASO that targets SEQ ID NO:267
tgagagattaatctagattgcct

SEQ ID NO: 288 - DNA, 23 bp, Artificial Sequence
  ASO that targets SEQ ID NO:268
agatgtgattcagttaaggagct

SEQ ID NO: 289 - DNA, 21 bp, Artificial Sequence
  ASO that targets SEQ ID NO:246
tgagagattaatctagattgc

SEQ ID NO: 290 - DNA, 21 bp, Artificial Sequence
  ASO that targets SEQ ID NO:243
gttgttttaagtcactaagtc

SEQ ID NO: 291 - DNA, 21 bp, Artificial Sequence
  ASO that targets SEQ ID NO:245
aggagcttgaggtgagagatt

SEQ ID NO: 292 - DNA, 21 bp, Artificial Sequence
  ASO that targets SEQ ID NO:248
acacatttctgttgttttaag